SEQUENCE LISTING (116 sequences)

SEQ ID NO: 1 - DNA, 45335 bp, Homo sapiens
aaggtataggtcatccaccagtcactgtggctcacaatggcccaaagcaggaagtaggagaaatcttcccctacctgagtgaaggaacctcgtggttatattttcattcattcactcacggatgacaacctggatttagtttgcctgggttgcattttgggctacatcactttttacttttgtgaaattgtgctaagccctctacatacagtgtctcacttaatcctcaagactaccttcatacacccattttacaagtgaagacgctgaggcttagaaagggtaactatattgcccaatgtctgacacacagaaatgctgctcaatctgttgctcgtttctcagtactcacttcacttaagctgtcattggcacttgcctgggctgatcatttcctctttcatgaaatttctttcttcagttcatgtcaagtacaacacactcatttctcctccaacctcattgtcttctcagcttttatatcttctgaatctctacgtgtgttctttgctacttatgcttactttataggggatctcctccagcctcatgtctttaaatactgtttctaatctgattagtcacaaatcatatctctagcctggagcttgaatacatctatatatcctagtccctactcaacatctccatgtggatgtttttccaatcttctcctttttgttccaaatcctttccctgtcctacctctgctctgctctgtcttgtggagggtggagtggtctgacctctgcaggctcttgtgtcagccacctttagcctgcctaggatcctcagaagggacttgtggaagactgaaggaagaaggaagaaagaaggggaggtccagctatttctagctcctctctttcttggccatgagtagtgtctctgacagagactgttgctcaacttctggtctatggtatgacacttccttcatccaataatgccccagctctttccactgttgctaataatttctgtggtgccacccaaaccctgtttgtcttctcagcaatgtcattacctgtgtaaccatgtcctctgtttgcaatgcctagagtggcttctatttcctgacatggcctgagttgacacaggtctctaaaaggcatctcaaaaccaacacatctagaacaaactcctaatttccctctcaagttgttcttcctggtagtttgtccattcaaattcatggcaactccattagtgccatccttgatgctactgtccctctcatacctgtattcaatccaccagcaagtcctattggttcaactttataaacatacctagagctgcctatttttctccactttcactgctatcaccatcctggtccaagtcattattaactctcacttggactattgtggtagccttctgatatggtttggctgtgtccccacccaaatcattatcttgaattgtagttcccataatgcccacatgttgtgggagggacccagtgggagataattaaatcatggggggcagttttccccatacctttcttgtggtagtgactaggtctcatgagatctgatgggtttataaggggaaacccctttcacttggttctcatatcctctcatgtctgctgccatgtaagacgtgccttttgccttctgccatgattgtgaggcctccctagccacgtggaactgtgagtcaattaaacctctttcctttacaaattacctagttttgggtatgtctttattagcagcataagaactgacacagtaaattggtaccagtagaatggggtgctattgtaaagatacccaaaaatgtggaagtggctttggaactgggtaacaggcagaggttggaacagtttggagggctcagaagaagtcaggaagacgtgggaaagtttggaacttcttagagaactgttgaatggctttgaccaaaatgctgataatgatatggacaatgaaatccaggctgaggtggtctcagatggagatgagaaacttgttgtgaactgtagtaaaggtgactcttgctatgttttagcaaagatactgggggcattttgtccctgccctagagatttgtgaaactttgaacttgagggagatgctttagggtatctgggggaagaaatttctaagcagtaaaacattgaaatggtgacttgggtgctgttaaaagaattcagttttaaaagggaaacaaaacataaaagttcagaaaatttgcagtctgacaatacgatagaaaagaaaaacccattttctgagcagaaattcaagctggctgcagaaatttgcataagtaatgaggagccaaatgttaattaccaagacaatggggaaaatgtctgacagggcatgtcagagacctttgaggcagcccctcccatcacaggcccagaggcctaggaggaaaaaatacttttgtgggctggctcaggtcctccctgctgtgtgcagcctagggacttgtgtcccagcctgtgtcccagtcactctagccatggcaaaaaggggttaaagtacagctcaggccatggcttcacagggtacagtccctaagccttggcagtttccatgtggtgttgagcctgtgggtgcacagaagtcaagaattgaggtttgggaacctccacctgtatttcagaggatgtgtggaaaagcctgaatgtccaggcagaagtttgctgcaggggtggggccctcaaggagagcctgggctaggtcagtgcagaagggaaatgtgggattggaggcctcacacagagtccccaatggggcactgcctagtgcagctgtgagaagagagccactgtcctccagaccccagaatggtagatccaccgatagcttgcactgtgcacctgggaaagctacagacactcaacaccagcctgtgaaaggagccaggaggaagggtccccctgcaaagccacaggggcagagctgcccaagaccatgggaacccacctcctgcatcagtgtgacctggatgtgagacatggagtcaaaggagatcattttggagctttaagaaatgactgccttgatggatctggacttgcatgcggcttttagctccttcattttggccagtttctcccatttgaaacaggtgtatttatccaatgcctgtacccccattgtatctaagaagcaactaacttgcttttgattttaccagctaataggtggaagggacttgccttgtctcaggtgagactttggactatggacttttgagttaatgctgaaatgagttaagactttgggggactactgggaaggcatgattggttttgaaatgtgaggacatgagatttgggagtggccaagggcagaatgatatggtttggctgtgtccccacccaaatctcatcttgaattatagctcctgtaattcccacatgttgtgggagggacccggtgggagataactgaatcatgggggcagtctagcccatactgttcttgtggtggtaaataagtctcacaaggtctgatggttttataagaggaaaatccttttacttggttctttctctcacatgtgccgccatgtaagacaagccttttgccttctgccatgattgtgaggcctccccagtcctgtgaaactgtgagtcagttaaacctttttccttcataaattacccagtcttgggtatgtctttattagcagcatgagaacagactaatacaccttctaattagtctccctgcttctgtagttcagaatgatctttctaaactagattatgctgcccttctattgaaaatattacactggcttctcatcttactcagaagacacggtaagatcttgacaagtggctcccaaggcaccagacttctaggacttcatgttctattactttctactatgtacattctgttttagcctcaaagacctcaccactctggctaggacccaccaggaatattcctaccacagggcctttgtacctgctattttcacccagaatgtgctttctccagtatctaaggctgttaactacctccttgcctgtgctcaggtgtcacttcataagagtcctttcctgattaccatatacatatcatcctccttatccctgtttttttttttttttgggggatggagtctcactctgttgtccatgctggagtgcagtggcatgatctccgctcactgcaacctccacctacctggttcaagccattttcctgcctcagcctcccaaggagctgggattagaggcacacaccaccacacccgctaattttgtatttttagtagagatggggtttcaccaagttggccaggctggtcctgaactcctgtcctcaggtgatccgccctcctcagcttcccaaactgctgggattacaggcgtgagccactgtgcccagccccttgttgtttgttgttgttgttgtttgtttgtttttgttgttatttatagcacttatcaccacctcatgtattatatagttattcatgatgtgtctcttcccatcagaatatatacaagagaacaggaaccttgtttggttggtttctatacctccactgtcttactaatgcctggcatttagtaggtgctcaataaaatctttgttttagaaaatgattaataaataatatgtacatattgaatattgtttcctcttttaacaaatctttaataataataaaaaatttaaagcagcaagattgctttgcatcaggcacaatgataagcgctttaaatggagcccatgattcactttcatttatagagcccacatgctctctgtcaggcgcagaaggtgccagccctgcaggagcttcgggggcggagcagaaggccgcctaaaacagcctttgctaagagagcatgcgtaggcgacgcgctggtagagctgtggacctgccagcctgcgaggcggaggacgggctccatctcttagaaacgcctacggcgcatgctctatggggtcaactggggggctggcaagcggcagcgctggtctggggcggagtctccgaggcacttcccggtggctggctgctctgattggctgaacaaatagtccgagggtggtgggcatccgccctcccgacaaggcagaccaggccccctgcaggtcccctatccgcaccccggcccctgagagctggcactgcgactcgagacagcggcccggcaggacagctccaggtgagagtgccggcgccgcggcgctgccaggtgcgggtgcggcgtggaagctggtgccttcagcaaggggagggtcgcgccccgaggctctgccggccggcaagaccctctgcgtttggacccagcgagtcttgcctggggtgcctgcgggtgctttttctgcgggcgttggctcaaggcatctgccaatcaaccagggcaacttcagagagcaacttcagagagaagcccactctcttccctgggcgctgtccactttcctggacaggcgcggtctccggtcagtgaagctgcccagtgctcctggaccgcgaggacagcagggatgtctgattcctctctctgcatcctcgtgctctccccttagctcctccaggcatcccatcctacctctcttgttagccaacggcgtggtcgaaagcagacttgggcatcaatcctggttaaagtcccagcgatactgctttttaaacagctgacactttgcttccagtacttcagctgggtgaacctcagattcctgaaaaatggaattaatggtactgccatagggtcggtggaggagtaattgacagtagatcagaagcgctgcctgcggtggctggcgcctcgcaagagcttaaatgggagtcgtaataattgttgttattagaaagttaaatagcatgctgcgttggggccttttcttctgccactgaactgttggactgctcttatcttttttcagaatctgtactatgcatctaatatatacgtggcactttgcttggtcctgggggtacaaagctgagtcagattgttacaaagtctaaataactgagataaaggtgttaatacagcccagaaaagcacacttaattcagcctggaaggttttttttcccccctccttcgagattttcagagtatctccttcttagtaagaattgcttggcttagtatgttgagaaaaacatactaagtatgtagtgttgtgttggaaggaaaacttaactgggtgagattttcagctgagtagctttagctgttactctcatggaccttcctctgggcggttgatacaggttctgtaggaaaaccgctgaatgaaagcagctcctgcagatgctattctgttttcaattgtggtaattcctttaattttttccctttcaatttactccttattctctagtaataatggacttttgggaggttgcatttatgataaaaaatagcctggggctgtacaatccttcctatcatgaagaagtacaaagttgtaagtagaagcagtggccttgctttgccaatgcacactgaaggggataacgtttgctcctgttcagagttcacggtcgggcgcggtggctcacgcctgtaatcctagcacttttggaggccgaggccggcggatcacgaggtcaggagatcgagaccatcctggctaacacggtgaaaccctgtttctactaaaaatacaaaaacaaaattagccaggcgtggtggtgggcgcctgtagtcccagctacttgggaggctgaggcgggagaatggcctgaacccaggaggtggagcttgcagtgagccaagatctcgccactgtactccagcctgggggacagagcaagactgtctcaaaaaaaaaaaagaaagaaaagaaaagaaaaagaaaaagaaaaaagaaaaaagaaaagaatttcagagttcactacttcttggaaaaaaataatcccaggaggaattccatcaacagaagggagtcagttcacttttgcaatctcctgagaatcaatttcttaagacttgtggaatattcagtttaaacacaaaactaaaaacaaacagctgtattggagtattattgacatacaatagcatggcatatttcaattgtacaattgtataagtttttgcatgtgtacacacctgtgaaaccatcagtacattcaatattatgaacctgtacatcacctccaaaatattatttgtgcatacccctttgtgatcctttcttactgccccttctctccaggaaaccactgatctgctttctgtgaatatagattagactgcattttgtggagttttatatgaatgggatcagacagtacttttttttttccttattttgatctggcttcttttggcataattattttgagattaatctatatttttttgtgttccttcccttttatagctgagtagtaggtatataccataatttatccaatcatccatttaaagcatttgggtttttccagtttttgtctattacacccaaagctgttataaacatttatgtacaagtctttgtatgggcatacatgcctgcatctttttaagtgattctatttattttttaaaaatgtagaagttctgggatctagtcctaggtttttctctgaccacttgcgtgatcttaggagaggcatttaactttgccttcagcatcctcaaggtctttgctgtgattttgagtcatcactgccagaaaccaagatggtctttgttgatttgatggttagagaatttcagaaggagagagacgggtcaaatttttcctttctgtttcttttgaaagctctattttctttcatttcacttttttcccatgaatgacaaatttaaatgctgacgtataatttggggcaagaacagaagtggttagataagtaggaggaagagctaatagtggttagataagtaggaggaagagctaatagtggctgaacttaaacatgtctaaatcataggtggaaggcaagaggtgggaaataagcaagctcagaaattttgtgtctctttctttttctcaaactatcctcttctaagtcttgccttttgttatttctccatgaccaatttgtttttaactttggggttaggaggtgaatgggtatgctggactccaaaggtcatatatcaactggaggtggtgggtgggaggtttcttagttaaaagtaattcctttgggccgggcgcggtggctcacgcctgtcatcccagcactttgggaggccgaggcgtgatcacgaggttaggagatcgagaccatcctggctaacacagtgaaaccccgtctctactaaaaaaaaaaaaatacaaaaaaatttgccgggcgtggtggcgggcgcctgtagtcccagctacctgggagtctgaggcaggagaatggcctgaacctgggaggcggagcttgcagtgagccaagattgcgccactgcactccagcctgggcgacagagcgagactcagcctcaaaataaataaataaataaataaataaataaataaataaataaataaatataaaataataataataataattcctttgggaggcggaggcgggcaggtcacgaagtcatctgagaccagccttgccaatatagtgaaaccctgtctctactaaaaatacaaaaattagccgggcatggtggcctgcacctgtagtcccagctactctggaggctgaggcaggagaaccgcttgaacctgggaggcggaggttgtggtgagccgagattgtgccactgcactccagcctgggcaacagagcgagactccgaaaaaaaaaaaaaattcctgggcaggcgcggtggctcacgcctgtaatcccagcactttgagaggccgaggtgggcgaatcacgaagccaggagttcgagaccagcctagccaatatggtgaaagcccatcgctacaaaaatacaaaaattagccaggcgtggtggtgcgtgcctgttgtcccagctacttgggaggctgaggcagaagaattgcttgaaccaggaggcagaggttgcagtgagctgagattgcaccactgcactccagcctgggtgacagagcaagactccatctcaacataaataaataaataaataaataaataaataaataaaataaaaataaataaataaaatttaaaaattccttattctcctttcagatatggctggagtcatttgtttcatattttcctttcaccttttacttgccctaaatctggttcagaactttttgtgggagcattaagttaccagaatatttttgtgtagtaaaattcaagcaaataataatagactgttttattatacagaatgaaaatgcggttcttggggttggtggtctgtttggttctctggaccctgcattctgaggggtctggagggaaactgacagctgtggatcctgaaacaaacatgaatgtggtaagtttctcaaagttatgtacttttaaaatgcatctatttccccgatccagttatgtgagctacatgaagccatacccatacattcatctctttataactcctttgcttttcaatttctctgaattttttttttttttttttttttttttttttttgaggcagagtcttactctgtcacctgcccgcccaggctggagtgcagtggtgtgatctcggctcattgcgacctccgcctcaggggctcaagtgattctcgagcctcagcctcccaagtagctgggactacaggcatgcaccaccatgcctggctaattttttttgtatttagtagatacagggtttcaccatgttgcccagggtggtcttgaactcctgagctcaggtgatctgtccacctcagcctcccaaagtcctgggattacaggcgtgagccaccgtgcccagcccaatttctctgaatcttagattcaatttgctaggcttttctcaccaggtaatatgtagccaacaaacctcagtttaatgttatatatacttcttaataagataaattatgattttttattaattcatttcatttttaagtctcactaagtttgtttaaactataaaaaagtatttaaactttgcaagaacattaccgctattgagagctggttttagaatacattgaacaaaggttgtgattaagaatgtccagggaacatatttctatacagaagtcaaagtatctgagctaaattttgttagaaaaaatatgcaaaaataaccactcgactaattaaaacatctgaaagttattaatcgtaagtaaacaataaaatctggaaatacctcatttaggcacaaaatatcagtaaagtacaaattattaaagaaaacagcaactttaaaatagatgttgcagtgacccagctgaagaatcaggcaagcttagggactcgcagagaaattcacagtgagccaggagagtatgacctgataacagggtcttaaagttaagttttacttcttagctcaaagcttttcttttgcctcccaatttttccaacttttaaaataattttttaaataagaatgataaaattaaattctttcaagtacttatggtagagagggtatgtgcacttatttacattaaatcccctggataattgaattatccatatcccactgagctcctgattaacaattgtcccatgaatccttccttatagaaaagtgaatgtgaccataattccctattttgttcaacttttcttccatcccaccttgacattctatgtaattattttagtccattgataaaaacaagttaatattttttatactttattggccagcaggggatctctcaaaacaaagaaagtgttttatacattgacagttctagctgggatatttagaaaagaaagaaatgagcatttatttgtgcccggtaggcaggatggaaacaaagaggcccaagatgtggtaaagttaggcaaatttggtataaaacatgctagaatgatgaaactgaagtttaaccagacaccggtaggtggaacacaagcgtgaaacaagggaggatttactcttgtgatgagaagttggctactagatttagcagactaaaatttcaatgacaaatgctttcttaaagcctggagaacatagtttatctgctcctttgcttgttaatgtgggagactgtttcagattctgtccacccaatttccatcgtcctttttctctacagagtgaaattatctcttactggggattccctagtgaggaatacctagttgagacagaagatggatatattctgtgccttaaccgaattcctcatgggaggaagaaccattctgacaaaggtatgggaaggctcttaaaagtaaaaaccagaattcttctgggttttgtgttagtaaccaagttcagatttaacttaaaaacattgaaatgggattatttttagacgaaagcactaactgtgttgaggtttgcaaggcaaagaaaaataattttcttttaaagaagtaaggacaagagtcatctaattttttgttcaaaggccagattcatttgaggatatgctaaaatctctgaggctttgtttttttaaggaagtgtatttaatgaaatgttttgagcattaaatagatggcttttgctatttaaaaattatttaatttttttgaattcacataatctaaaaatcagagaattagaagacatacagtgaaatgcctcccttctctctcactccctacttcctgcttttttgtgtatctttttgaaataacttcattttaaaatctttttatttccaaaaatctcattagaaacaaatacacacacagattcttatttttcccttgcctccatttgaatgcaaaagatggcactattttgttctttgaattttttacttaatatcttggagatctttccatacaatacataaagcacatcttccagaagtgcatgatatttcactatatgactgtatcattatttatttaccaagtgtcctattaatgcatatttgtgctatttcagtcttctgttaacaaatattgctgctgtaaataagcttttgtgtgtgcaagtttatttacatgatacatgtccagaagtagaatttttggatcacaggatatatgcatttgtgattttgatatatattgccaagctgattttcatgtgcatgtgtcctgataattactatcactatgaatatatgaaagttcttgtagctttggcttaaaaagtatatatatacatatatatgtatatcttttttctctctctcattttataactcaagcaaaactgcagtttccagtgcagataaaggaatttctagacagactgattgaaattattaagcagttatcagaagaaggatctaatctttattcaattatctctccagatgtagcttatttatttatttatttttttgagacggattttcggtcttgttgcccaggctggaatgcaatggtgcaatcttggctcactgcaacctccgcctcccaggttcaagtaagatgtagcttattctctaacatttttttgtttctggagacttttcttggggaaattaaggtttagattaaggtagttagatagctatttattcttcaatttaagtgttatacgggcaaaagagccacctttgcctcaattcctgcccaaattgttattcaaagcaaacttaaacaccctttgtgttaatgtgagcattgtattattatttgctttgttttttcttaatttatcctagtttaacttttttcttccatgtgttctagctgtaataagattttaataatgtttaggtggccacgacaaccattttttttgaaatttgatttctaactcttgagatttttatgctttacagatatatgccattaagcttcctttttataaatatgttataaaatgagtgatgagttatagtgagagcttttagtttttcctgtgctcaaaacactcaaaacatatattttatgctagagcgagcatatgatcttggtttccttccttttttttcccccaaagaataacccctgtatactgatttcagacactgtataaagaaaaatgctttatattatttttcttaaaattatagttgattaattagtaacgtgaatcactgttaaatgatggtaagcatctcagtaaaacttgctgagagctctcctcctatatccagcttcagacttctctctccatacagtggttaaaacactcagagtcacatgccctacctgtgtgtatcacactgttgcaggggaaggtgcagtgccctggaatatgaagaaagtgagcatgagtcttgatatgctcccaggtaatagaaaggagacttatgggaccttggtgctacacctgggtttgcccctgacttcttgtgtgctttgttttttcttttttttttttaacctctgtgggatatattatatatactttattttatttttggcaagagggttggggattgggagagtggagagtctaggtctttagaatgggggaatatagactgaaagtatttactctaagctaaatctgttcatagcatttggtcctcatgttctttctctattctgagaggccctccctgtaggggatcactgatgtgtatgtcatacctgctatgagctaacctcagtggggagtcttcaaggaagtctacagtgaacaactcagttctctaaaggaattgtgattttcctaagaactgcataggcgctttcctgatggtttgcatttccacattctaaaacagagcatggacagaggctctttactgtccttcactcctattcaagagcaaggttgttgccactggtgttatttccggtgtttaaaggcaaatagataaaaagaatggagaaggcctttcacacccaaagtgaaacgcgtcctcttctcatgcatcccgcttcccctcctgacttcctcctctattattaatagcaccagcatcctcctaatgacctagcctggaatctgcagaatcattcttgaccacgggtcctctctaggccacccacgttaaattgcttcagttcatctttttcttctgtttccctgaccaccagcctacattaggctcttgttaccacttaactgggcgcctgaaataagctcagcagctgagagagtcttgagccatttccattttcaaggctcctcgtatactaacagaaagaaagaatttcaagacaggattatggaaattcagtttgcttatatagtttaagaaattaatgcttttaatacacacaaaaatactgctatacattcatactatttacaagtaattacaaaatgtattaaaacattattcattttttggtaccacattccttgtggaacctgagtctacagctgaatggtgagcatctttcagtcctgttgccatgggcctctttgactgactgtgtagcctcacttggagatcaggttttttttatgaacacagggcttggccacatggcttctctgctcatgtttgcttaaaggctttacctaactgctcttttctctgccaggcttcctgttctcattcatcctatgtggttttgcaggattaagtttcttaaagcttagctataatgatgtgaaattgccattcagcaatctttaggggtctcagaaatgaagatacacactgttccttaaatattctgtccagctggtatttgtacctttcacctccattctgcacagtagtagcccttggctactcgtgcagtatgtgcatgctgtttcccaccattaagcaaccttcttaggaagtgttgagcaatgctttgctcctgaagggaattgaaattcagaaattacacttcatagggaagtggctctgttttcatctgctcagccttatttcagaaagtcatggggagttagcactggagggatctcagattcctgcctgggctagggatttttgtttttttttaaacacacttcactggtggattccaaggtagaattcttggttttggcaacctgtgttctagactaacatcctcattttgttggataagacagccaggttcaaatagggaaatgaatttgtctcagggcagagggtcatgtcctgacctgtctgactacagattcagtgatctttgaactggagaatgtggccttacataataaatattaaccaagttgatgccatgataggcacaatatatattttatatatatatacacacaccaagagtggcacatttgtatctgaaagttatgcatctgattgccgagtgggtgaagtggcactctagactgtgaaagcctggaatgactgccttcagcatgcagaaacaggcttttcaactgtggcctcagcaaagattccccaggggttgtccattgagctattagagtcatagctgaaaattcacagcgtggcttgtgagttaagtctttagaaggagaccatcagcttgtattttgctttgattgtttattttagaattctggttgatttctagatttttctttccttatagaaaaatccttcacttctaaagaaactattttttgaatcactgcagtgctctaacgggctacacataggcgttggtaatctgggatgtaattcctcacccctggtggtgccgaacagacatggtttcattaatcaatgcctgagcttggtgacttgggctgagctcaggctcatgtagcactctgatggcgcattgggtaggggattgcattcagaacctttgcctaatggagcagcataagtgaggtcagctgtacagcttatctcatagacccaggttgggatcagagcagaaagccaagtggaaaggaagatagtgctctgccctgtccccttccctcctcttcctcctggttaaccgctctgacctgtcctctgttctcaacctaattttcacatcttcagggaggatttcctcagcctctcacaccaggtcaggcccctggctacatattcttatagtcctctgtgtttttctccatcgcaattaagacaataaacaatatattattatgtaggtgatgatgtgtgtgacctctgtcatccccaccagactctgcggggatgaagacttttctgattctctttacttggtaaattgctttagctccaggctgcagtctacccaagcatgctcctgcagccctcctgtgacattgcgatttaatagtgtcccctgctcaagggcacactgcatgaaggatttcctactcctgggctcctaggagtccacgaacacaacttaaatgcttcctgtggtgggtaaaacttgaagctgtattgcctcatgattaagctgagcatgtgaagagccaccagtcatgggtgtaagttctggctctactatttaactggctgtgtgaccttggcaattttctttacctcttgaagttttgacttattcaattattaaacaggaagaataatactggctacttcgtaggctgttgtgaaaatttgataaggcctgtgaaatatatgcagtatagtaagtgcaggtgatacaatttcttttacaacatttatttatttaaaatattaatgaataaatacataaatattttaaaagtctcaccgtgtctcccaggctagaatgcagtggtacaatcatagcttactacagcttcaaatgatcctccctcagcctcccaaagggctgggataacaggcatgaaccatggtgcccagcctctgtttttaattttgaaatgtcaacatggatttgatggtggtctctgatattgacttcttcaggattaagccctccatgacagggatgtattctaaactgtgtggacaggaggaagatgcatttgtttacaatggacagaagtaagaaaagtaagaaatgtcatagaactatgtctgggggtcaggtgacaagcactgtgtcatttctagccctgtgtcccctgcaggtgctttcttctgtggaccctctgctccttctgttgagggagaattttggatgagagctggataatttcccaaaattcgctcaactgtgaagctctggaactcaagttgctgtgctgcaaagctgtctgaggacaggccgtggtggcagagaggggcagggaagacaggcaatttggcatggagatgatgggcacatttgtcacatgacctgggtttgattcccagctgcatcgtatactaattgtgtgtcctagggcaaatgatttagtctctctaagcctcagaggccccaaaggaggaagaatgatgtaaatggcccagcaccatcctgggtgcgtggaaaactctaattatgataatgtcctggaacgttctgcagtgaacaagacagaccacattactgccttcgtgttgcctggtgatcagagtgattattgctggttttggggaaaacagcattctgaggcatggaacggttttcttccctgctctgtgatgcagggtgctcttgttgtgttcactcctgcaggtcacctgctccccagcagggtgacagtgaagtgtgagtgcagtggactgcacagtggcaaaggccacatgactcctgaatgcatcctgtggacaggccaagcgctgtgccctgtgtgaatcatcaggtgaactctcatacaattctagggtgtaagaactcctattacctcctgttttacagatgaggaaaccaaggtttatagggttaattaacttgcccaagataaagcagctgatgggtagtgaaggcaggagtcacacagcccgtgactgtttctgcccacttggccctcttctcaggctagagaacagctatgatgtccctgataaagtcacactgtggaaagtggaaagtatctgacttagatgtcctgcatctgctagatgtcctgcttgtgcctactacagcaactcagagagctgttaggtgacttatctgagataatgcgtgtaaagtgcttaagtgcttagcatggtgcctgtcacattgtcaacacttagtatgcgcttgctgttactatgattatattacctgggccaaagaatgccacttcactctatccttcttcatagtggttatgttctaataatattttattacatttacagttaactctttaacaacatgggggttaggggtgctgtcccccagagttattgaaaactgcatataacttttgcattccccaaaatttaactaatagcctactgttgaccagagggcttacctgtaacgtacatagccaattaacaaatattttatatgttatatgtattatattccatatatacaataagtaagctagagaaaagaaaatgttataaggaaatcataaggaaaataggatgtatttactattcattaagtggaagtggatcatcataaaggtcttcattcttatgttggaacaggctaaggaagaggaggaagaggaggagttggtcttgctgttgtaggagtagcagaggtggaagagggggaggaagtggaaggggagacaggagaagtgggcacattcagggtaactttatgaaaatacattgtaatttctgtccttttttcatttccctagaaatgtttctatagaatagcaattcttccatcatttgctttagtttcagtgcctgtgtcatagaagcgtcagtgttgtaaaggaagtcttgaataattgcaattgccacccttctcctgaattgtctaatatcagtttgttttctagcactgcatctcccatgtcttctttcccatcgtctggtgctgttttagaagcactcgtctccatcaaatcatcttctgttaattcctctggtgtgtctattagctcttgactttatttaagatgcatatattgaaactatttacctgctcccaccctccacctttttgccatatccacaatctttttcttgatttccttgattggctaagtcatggatatggatactgtgaagtcatgcacaacatctggatacacttttctccagcaggaatttattgtttcaggcttgataactttcacagctttttctgtaacaacaatggcatcttcagtggtgtaattcttccagaccttcatgatgttctctctgttggggttctcttccaattcaatcctttccatagtgtatgtgtgtaatgagccttaaaggtccttatgacctcctgatttagagactgaattaaagatgtgtttgggggcaagtagaccacttcgatgcctttggtattgaattcatggggttctgggtggccagaggcattttcctatatcaaaagaactttgaaaggcagtcccttactggcaaagcacttcctgacttcaggaaaaaagcatcaatggaaccaatctggaaaaatagttctcattgtccagtccttcttattgtacagccaaaagactgacagctgatgtctatattttccctttaaggctcagggattagcagatttataggtaagggcagacctgaccatcaaccctactgcatttgcacagaacagtagagttagcctatcccttcttgccttaaatcctggtgcttgtttctcttccttactaataaacgtcctttgtgggatatatatatatatttttttttgttagaatggaacactttcatctgcattaaaaacctgttcaggcagatacattttcctctaggaatttatatgcagcctcttggttggcaggagctgcttcttctgttatcttgacatatttttaaagtttttaaaatttttaattcttaaaattaattaatttttggttttttagagacagcatcttgctctgtcaccaagctggagtacagtggcaccattatagctcactgtaacctcaaactccttggctcaaccagtcttctcacctcagccttctaagtagctaggactacagacatgtgccaaccatactcggctaatttttaaaacaatttttgatagagatggggatctcactgtgtttttcagactggtttcgaacacctggcctcaagaggtcctcccactttggcctcccaaaatgctgggattacaaacataagccaccgtgcctggtctatattggcattttaaaagccaaacctctttgtaaaattatcaaagcatcttttattggcattaaattctccaactttagattcttcaccttcttttttctttaagttgtcatataatgactttgctttttcttgaatcttagagtctagaatctattcttgctttatcttgaatattagagtctagaatcatattagagcaatcctgcacccacataaaagctgcattttcaatatgagataaaaggtatttcggaaaaagtgcaaggttttggcacttgctgtcatagctgcaatgacaaatggcttcacaaattttctttccctttttttttaacagtcatctttatgtgtcatttatcttgaaatgaagataattgaagctgcagacctcaatctatgacacatatcaagcaattcaactttttcttgtctggacttttctctgctttttgggagcactcccagatcactattagttctttgtatgggtccaatggtgttattcaaggtttatgatatcgtgctaaacatgatgaaaaacatatgagaactgcaagagatcactttttactgcaatgtgcaatttactggagaaactgcttacgtggagatgattagtgtcatgagaaatttaagcagatatttacaatacttgagcaacaggaggcggctacaaaattattacagtagtacacaataaactctagttaattttatgcagttatgatttaataccacatctttacatttgtttacatttctcttaactgtcaatggcaccatgtttggtctgtaagtgtgtgtctaagttttgaaaatgttaactttttatactttgtgtatatttatggtaataaataaaaaaggccaatatctaaatatattttatgcattcatgacatacttaaccttttagtaactttttattatttccagactacagagttggtctgttagtttttctcaaattgttgcaaatctccaacaaatttttcaatgtttattgaaaaaaaatccatgtatgagtggacctgtgcagttcaaacctgtgttgtttaaggaccaactgtatagatttattattgttactatcagggaccattatttattcagctttactatgtattaactacttcgtcattctcactttagaaaaggacacctatacttcagagagagttgagcaaatcctacaaagttttgtagccagtaagaactgaggtctgcttcatgctaacgccctggtgtctgaccacctccttgttctctgttttggttctgccctggccctcagctgatttctcacatgtgttttggagtgtgacggagtcctgtccggactcagcatcctggcagagaaggccagtgccttgggaggcaggaggaacttccccacctacccggtcatcttcctcctgcgggactgtgggctcagcacattcagtttcggagcttgagtaatcgcctcctggcttccacccacactgggaaggcagcgttgtcgtggactgccactgggctgcttctttgtcagctttgtcctatttagggccataatgaaatcacttgccatctccaggctgagaaatggtcctttagtcttttcccttaatccccagccctcctagggcttctttttcttcaagttgcatttgcacagaactcccagagccacccgtagggcatagctggggaaggcagcccttgacctgtcatgctggtttgtcactctgacaaacagggcttcagggtgcctgagtgcattgagcaggctgggccttggaggagctgcctgaccaggcgaggctgagtgggtgcccctcctttcattccctatccccctccatctactagagattactttcttcaagcactttgtccacatcctttcaaaaatagctttctgccttcagccggaaggcctcagtgtgttctagagacttctctcttgttaaccttctttccctagcaaagcactgaaaagcatggcctgtggtgtcagacacacctggttagactccaaccctcacgcttcctagctctcgttcagccttgggcaagttacttgacttctttaccacaattccctgttctgttaaatggcagtagtgccaacttcaaaggattttcagtacagtacctacagcatacaagcgtgcaatcggcgctagctatcatttcaatcagctgttctcagcaaggtgaagtgaaatctgaaatagttcactggaagatgtgaacagcaataggttttaccttcccagagagaaacatcctttgccagtcaatccttaaatccttctttggtggtgggatgggaaagaaccacattgtaccttcctttcttccccctggtgggagcaggtgagagaagctggcaacagttggctgggcctgcttccagcgtccagtcttttccattcccttttgtattcacccaatgccagagggaaggggcaagaggtttggaagccatttttaaaggtttttcacccgaggattcgatactattttctttgctgaggatccgtcatgcctaatggaactgccatctctagaagttcagtaagttatatcctctgcatctccctcctcagaagagacttttttgttctgggatgaaaggaaactggcccagcctggaaagatgtgcaccccatgaagtccttcctattccctgtgtccccaggtcagacaaggcactggacttttcctagtggttccccagcctggctgcgcgtcagaatcaggcagggaggctttcatgtgcacactggtcccatctggccaaggcctgggtgcagtgtcagctctttattcatctggctcctatgttttctccagaaccctttgtggaaacatctgtctcttacttagaatccaggtcaaatgtttgtcccactggccaagtaactttctccttcttctgggacttcctgagtggtgttctagccttcttcttgctgggttagacagtcatcgtgtgtctgactccctcgaggatggagctccctgtgggagggagtgtgggttacccatctttgcattcctagcatctattagtttggggcaaaagtaattgtggtttttgccattgcttttttattttgtggggggtgggggtaaatggaatccctctctgtcacccaggctagagtccagtggtgcgatcttggctgaccacaacttctacctcttgggttcaaacaattctcctgcctcagcctcctgagtaactgggattacaggcacccaccacgagggtttcaccatgctggccaggctggcctcaaactcttgacctcaggtgatccacccgcctcggcctcccaaaatgctgggattacaggcgtgagccaccgtgcccggcctgccattgctttttaagggcaaaaaccacaatgacttttgcaacaacctaatagcagtggctggtactcagtaggcaccactaaaggtttgcacgaaacgtaatgtatgttttcaaatcacttcttgatattttaccttctagtgcactgcttaagatacagcagatttccattaaaaacttttcacctgaatgtggaggacacagctgagtggcctccattctctaaagccatgccatttgtcagacgtttacctttcttcatcagcccttttcctcatctgccagcatttcccatgagaaaacagtgtggtctgcagtaccctttgcagtgcttggcccagattctccagatttgaaaacatttagcttgtaaagaatagattcactgtttccttcccatctgctgcatcttttgtgcctgttgttcattgagcttttgtattcagcttgaagtaggggatggaagaaccctaacaaacaaaaaaggacaattagaaaaatagtgtctttgacttattctctggaagcctccttcaaaggataaagtaattccctgagaaaagcgttgtgcaggtgtgaattcggtctgggtggaacaagggtatttaccgatgttcttagaagctgcacagccaccttaggcgtggtctcctgggtagcacatgctccagctgtccacaggccattcctttcttcacactcacctcccaggaaaactgcagcagctcctcattgcccttgggaatccaggagttatttattggggatgtttaaagactttcctgatgtatcttcaacttgtcagcttccacatttccccattcaaagctctagaggttacagagggaagtacagagactttagggaaattttaaacattctccatcgagagaaggtggggagctgagtcctggtcccatgtgctggccaacatctccccttccagcctctgtcctggcctggctgccacatatcaccctccttctcctctctcaattatccttgccccagggccctctttggtttcaaaccattcctgcctttctctggacactctcccctgaggagtagactaggcagtcccatgttttccgtgttaactacagaggcacagattggaacaagtgccaaggaattaggccacctaccgagccttcctcttgcctctctctgactctccatcctgagggaagacgattgcagtcgtttttgttggaaatgacctgacctgtgggagcagcccgttcctggcctttcaaagtcaagtcaccttactcatggcctagctcctgatcactcctggtgggtgaaaagtgcctcaacatacctaaagagctgtgacatgagcaggaaagcccctctttctagaaagtgttgggcaccatcagtacagcagagccactgcatcgtacagcgcccggtcaccattctcacgtacagttaccatgagatgatgatggtgcctgtagttacacatttttcaactataatttaatgataaattaattttggtaattataattgtcacttacactgcagtttataattttggatttttaatgatcaattttcatattttcccagctggattatcatcctcttcattgaagccaagaagcctgtgcataagacgctgtatctaccacaatgccttccacttacatttttcgtagtaaatttagtcttgttaatattgcattaaaatgaacctttgaaggcaaagatgagcacataaagcagccctaatcttgtccagtttgctttctattttctccccatgggtagaagttcacctgcatggccctggcatttctattttgtgattagaaaatcacaccagccatacatgatgtttcacttctgtaatccccgcactttgagaggccaaggcgagagaatcacttgaggccaggagtttgagaccagcctgggcaacatagtaagacctcatctctatgaaaagttttttaaaaaattggctggatgttgtggtacacacctgtaatcccagttcttcaggggcctgaggtgggaggatcacttgagtccaggagtttgaggctgcagtgagcagtcatgccactgcactccagtctaggtgatagaatgagaccttttctctcttaaaaaaaaaaaagttatgggaatcattttatactcttggcctggggtcctgctattttaacaacacgagggtatagcacccccaggttgtacaactccagggagtaccatgtatattttcatgtatgtgagcagtgccttccggagtagtctacggaagctaccttgcagcagaactgtcatttaaaaacaatttgctagttatcaaatctatattaggacattagcagaaatgaacatttgtcaattcacttatagtcttatcatctggacaaatcattcttctcatttttccttggtattttacactcctctttggtgtacattaattttgcacagctgtaatttagagtagtctataattttgtattttgcttttttttcctttgcaaggtataagcttttctatgctgtcactgtccttgtcatggttgatggataattattataccaagtggatttcaatgaattatttgaccacttgacaattagcttatttccagtttttcactgttataaagaatgagcagagggcaaaggaagctctcatttgttcactgtgacccgtgtgtcagtcactatactggatacttacctcacttattcctcttaacaattatttttggaactgagacgcagactggttaagcaacttaatagtatgagttgatatatagtgaactcttactgtgtgtcagactctttgctacctacttttcatataatatctcatatctcactgtgtcctataaaatgtgtattatcatttctactcttcagggaattatattaattttaggagatgtttccccaaggtgtcatgttagaacttggagaggacagagttgagcctaggtttgtttgagctgtctaaaactcatgatcgggctgggcgtggtggctcacacctgtaaaccccgcactttgggaagccgaggtgggtggatcgcttgagtccaggagtttgagaccagcctgggaacatggtgaaacactgtctctacaaaaaacacaaaaattagctgggtgtgttggtgcacgcctgaaatcccaactactcaggaggttgagatggaaggatcacttgagcccagaaggtggaggttgctgtgagcagagatcacgccactgcactccatcctgggtcacagagcaacatcctatctcaatcaatcaatcaatcaaataagtggataaaactcatggtcagtataatgtcagctccatggaggtactttttttcctcttgtttttgttctctgctgtgttctcagtgctgagaagaaggcctggcacatagttggtactcaataaaggcttgtttcatgaataactgaatggtctcagcgttaggtaagcagcttgaggtcaggtttctttggctcccacatctttgttttttctgctatatcactgtctcgggtacaggtgagtagaaaataaattctcacaggcagacacagacacatatatttatgaatttgtgtgtgtgagcctttttatctatttaacttttattcagctttaaaaatttttattaaattttattttttaaagcagttttaagttcatagcaaaattgagtggaaggtacagagatttcccatacaccctctgcccccacactcccctgttatgaacatccctcacagagtggaattgtaattgttacagctgatgaacctacattcacacaaagtccgtagtttacattagggttcccttttttttttttttttgagactgagtctccctctgtcccctaggctggagtgcagcagtgcaatcttggctcactgcaacctctgcctcccgggttcaagcaattctcctgcctcagcttcctgaatagctgggattacaggtgtggggcaccacacccagctaccttttttgtatttttagtagagacagggtttcgccatgttggccaggctagtctcgaactactgacctcaagtgatccactcgcctcagcctcccaaagtgctgggattgcaggcatgaaccaccacacccggccactacagttcactcttgatgttgtatattctatagttttggacaaatgtatgatggcatgtgtctacctctatagtatcatgcagagtagtcttactgccctgaaaaatcctctttactctgtctatgaattccttcctccctcctaacccctggcaaccaggatcttttttactctctctatagttttgtcttttccagcatgtcatatagaatgcagcctttacagatcttggtaatatgtatttaaggttcctctacatctttttgtggcttgatagctcatttccttttagggctgagtaatactccattgtctggatgtagcacagtttagttatgcattcatttactggagcacatcttggttgcttctaaattttggcaattatgagtaaaactactatagacatctgtatgcaggtttttgtgtagatgtaagttttcaactcctttgggtaaaataccaaggaatgtgattgctggattatatggtaagagtatgtttagttttgtaagaaactgccaaacttttccagtgtggtgctaccagtttgcatttccaccagcaatgaatgagagttcctgttcctccacatcctcttcagcagttggtgttttcagcgtttttggatgttgctcattctgatacgtgtatagtgatgtctcattatgtgtgatctttttctttccttttgtttattttgttaggaattattgtgagaaatgcaattacgaaagcaaattgtaaaaacattttataagaaaaaaattgtaaaaacattttataatttcttaaaaatatattgatttttgtttttgctgcttacataagtgtgtcttgttagtggacatgcaaaaaatggtccagtttcactgctaccttgccagtgctgttttaaaaaaattctttactaacattatatgtgagtacatcactatgtcaatctttcaatttatttctagttcattatttgcttatggatcttttacgaatgttctatttagcaagatatcatatttgttttgaagcttggtgctactgcctcctaaacaatgaatgtttttcaggtcccaaaccagttgtcttcctgcaacatggcttgctggcagattctagtaactgggtcacaaaccttgccaacagcagcctgggcttcattcttgctgatgctggttttgacgtgtggatgggcaacagcagaggaaatacctggtctcggaaacataagacactctcagtttctcaggatgaattctgggctttcaggtatatatgaattgataatggcatggatgtatttccttagtactcttaaagcagacaacaggcttccagcagaagaggtagataggtggtaactctgaagttgtatgagaggggaagttagtgtcttttgaaaggttttaaatgttgctaggaatttaatgactagcagtaaggtaaattataagtaaatgattacattaagatttacatttagttaggaattcttaagttacttcggcatttctggtggtgtgggtgctgctgggtaaacgttattccataactttcctcctttctccataaatatgtaatccagatgttcacttttcttctttccagaaattatcctttcctccctcttccttctggctccaccagttaattgctgtatgacattggacatcttacataagcctcctgtgtctcggtttccttatttgtaaaatggagcgtaataaacacctacctcataaggtcactgggggcttaaaggagagggtgcagagaaggaacctccacagaacctggcaccttgtatgaactggctaggggttggctcttctcctgccagtggcaacatgcgcatgcatatacccatacgcacacttgggttttggtctatgttttggtaccaggtattagagaaagtcagcagcactatagcagcctccgggcttgcttcccatttttaaaaccagaggcacctctaaggacatgaaacacaagaatgagagcttttaacaaaaggcatatacaagaattggtttattgtcacgctatttgtaacaacggaaaacaggaaactacccaaatgcatgtcagcagtgaatggataaagtgttgtatatttatatagtagaataagaatgaacaacttacaattatatgcagcaaaaaatggattaagccagatacaggagaatacacattgtataattctacttacagaaatatttaaaaacaggcgaaactaatttctggtgtgaaaaatgaggacaatagttacttttgggagacacaactggaagagagcccaagggggcttctgcagtcctgctggtgttgtttcctggactcactgctggttttcccagctgtgtttagtttgtgataactcatcaaactgcatactcagtatgtgtgtgctctattttatgtactatttcaatgaaagagttcctttcttggattacagttatgatgagatggcaaaatatgacctaccagcttccattaacttcattctgaataaaactggccaagaacaagtgtattatgtgggtcattctcaaggcaccactataggtatgtatgtaaataagatcagaagttgatataaattcttcattacagagtttgtacttttcttaaaagtgaaatataaaaagatgttagttcaaattccatttatttttaaatgccaaacagaaataatgaataagataaggaatgttggtaacatttagtcttctatgaaaattcatgtatatggttgaaattgaagaaattaatgtaagccacaatatattacatgttttcctagatgaagcaagatagggatgtgagagagtagaaaacaagtagtttctaagttcagagacttacataaagaaaacaagaaaatatactgccttctgaagttaagtggaatataactaactacaggtacatttggtttacaaaaaattttgaaaagttatatgcattttaatttatgctgattatataagtaatgcatgttcactgtagaaaaacagaagaacagaagatttagcaaaattaaagaaaataaaaattgcccataattctagtcagcaagagtttttgtaattagggactaggaacctgtcctataatttatttaattctcccttttaatgtttactatgactaacaccttagtgttcccatcacatgctttccttacaatatattcctagaaggagccttactatatgaaaggtatgaatgttttgaagggtctcaaaacatgtggttatattgccaggaaaaaaatggagttaggcatttttctttatttatatttcttctttcatgacttgcctgcttgggacctttgccccactgcttcctaaagtgacattctggggccaggtggcaagacgtcagagagggttgtattaattcgttttcatgccactactaaagacatacccgagactgggcaatttataaagaaaaagaggtttaatggactcacagtttaatggattgtgagctagggaggccttacaataatgacggaagagcaaggaacatcttacatggtggcagacaagagagagtgagagccaagtgagggagtttccccttataaaaccatcaaatctcaagagacttactcactaccacaagaacagtatggggggaaccgcctccgtgattcaattatctcccaccaggtccctcccacaacacatgggaattatgggaactacaattaaagatgagatttgggtgggaacacacccaaaccatatcaagggttaaaatatattttaaagctaggggattctaaatgtgtgagttcctttgaaatcattggtggtttatttcaacgtggtgtctgtaaattgttctttgggggaggggcatgggggagataaaggtgatggggtgaggctcttggaaatagtctgttatttacattgtaaaaggaactgggaagattttctgattctcccagactggatttcttccagtgcttatctaggttgagatttggagcaagcattaacaaatgcttgatttactagtttaaccaaattcagtgttagggcacacggaagttcagagtgccccatgtcaagtgttttggctcctgctggtggtattgtttgcgtgggtctcaggcctccgcgagagggcgtcgcgagtgacggcctttgtgttttctgagaaggaaatcccagatgatggaattcctgttttctgtcctttgttctcacaggttttatagcattttcacagatccctgagctggctaaaaggattaaaatgttttttgccctgggtcctgtggcttccgtcgccttctgtactagccctatggccaaattaggacgattaccagatcatctcattaaggtacttggacccctcccatccctctcctctccccgcagatttcctcctgagatctgaagaaatggcaaggggagggataatctgtgccttcctcccctgcgttttgatatcagtggagcagtgggcttttctttttccgtttacccctccttccagacccaggggtggccggggacgcctgtcgtttcctgcacactggtgccacgtgtactcatggttagcatgtgtcagtacagctctgcccacctcacagggagagcaaggagagtctgggagaaaataatttaagcatttgtggagttgccctttatcccatgaggtgagcctgtgcacagaggatgtaggagatgggagataggaaatgttcccaaaagcccatccctagctacactgagggtgacctaacaacgctatcattattgtattttataatatggctcctaaacacagccagtagcttcatcagggctctgccctaaagctgtggcacccgaacctctgttcagggaggaaatagatttcatgaaaaagagttttaagctaggtaagaccagatgttctattatgcatgcattatatgtcttttatgaaatatatgttcacacacacacatatgaaatcctgtatagaaatatatacatgaaatgaggtataacgtctctcatgggccatttagaagacttaaataggaaagatggaatctcctaggcttaggcagtccttctgccttggcctctcaaagtgctgggattacaggcatgagctaccatgcctggccaataaaaaaaattttaaaaaacaaaataaaaaagcaagcgatgggctggatttggcctgctggccagtagcattgaattagatgggcacacctctggcagacattactgatcaatcacagccttgactgtttcttagaagtaaccaccgatccatcagagtcagatgaaaattacttggggatggccctgggaggggtgatcattaaggccctttcccactgtagaagtccgctgaaaacttatttgattttctgcctccttctcttcatttggagaatttaaatacacctctgtagtgtgtgatttttgctttggtaaacttgtgcaaaagcatcctgatttgatgtccactggttgccattctctcctgaggccattcgtggagacattgggtacttgtctctgcttctgaggtgagtcacggagacttatgcaccagagtgaaatgctgagatgttcttgggtttctttttattttgtaggacttatttggagacaaagaatttcttccccagagtgcgtttttgaagtggctgggtacccacgtttgcactcatgtcatactgaaggagctctgtggaaatctctgttttcttctgtgtggatttaatgagagaaatttaaatatggtatgcatgtttatagtaagatttgatttttttttttatctgtgaatgtgcttatttgtgttgaatgatatgggagaggtgggaatgacctcatcagaactctaaagagtctttcatttgagaggcacaagcatgaactttggccaatgcctagaatacggtaccacctgctgccatcctcaggctgactgtggtcaccttcttatgttgtacctagatggagattattgaatgaggagatctgtcacaaattaagctgtaatatttataattactctagtgatttgtttcttttagaaatcattataagtataaacataaagatgggaacaatagacagtggggactccaaaaggaaggagggagagggacaagggctgaagcctgttgagtactatagccaatattgagtactgtggttatgggatcaatagaagcccgcatctcagcatcatgcaatccacccttgtaacaaacctgcacatggaccccccgaatctaaaatacacaaattttcaaaaattactataagtatttaaatttggaacggccatttacatacacataagtgtatgcttgcagtgtgtgtgtatttataaaagtacattttataaaatgtaaacccattttattttataaaatataaacccttggccaatactatgcccgtgtttctaaatatgtttgttagtttgtcatgtggcattttgaaaaaaagggttaaaatcctggaataaaggccctaaggaatttcagtgccatttttcttttgtctgataattaaagcaaggtgtgattgttatatgaaatttgggaaattcaggaaagcatgaagaacaaaatgaaaatgccctgtaaacgaaacccacacagtcccgtgcttaccatgttgttgcacctccttcatatggactttgtatgtggttgtatgtatttttatagggcagatttttagtcaaataatttttttctacctctcatctcacatacttgagattatggctctagtttttagtgctttgaagggcaaaatacaatgtttattatcaatgccaccttaatgctgttttcattcttcatttcaatgtatttattttgcagtctagagtggatgtatatacaacacattctcctgctggaacttctgtgcaaaacatgttacactggagccaggtaggcattccaggagtgcatttggggttcatgtaaaatcaacatcagaaaggtctgggcatgcaaaccctttccaaatagaaagacaacctgcttacaaatctgatctggttttcttccccagagtcctgggtttttgtcatcgtgcttgtgttgcttttgatacctgtggtggggcacactgtgttatacgtgggttcacaaacagctactggggttgacatttttcttttccctcctctcccttcctcaagtctcaggttaatatattctctccctttccttctcccccagctttcttttcctcctcctgtttcctcccctccatctgcttctcattgagtcctagattttttttatttctgtgttgcttcataaagagtgattttaagtccgttttggagatagaaaccgctgtttcaacactaacccctgatcacaatatgcttggaatagcagtgaataaactggagctaaaccaatgatagatgtgaatgggggcccctgacttttgaaatacagttttgattattttatcatgtaaataagtcatgttcattctagaaaatttagaaactacatctagaaaaaaattatcttaaaatgaaaataaaatcattctataaccctagacagagaggaaatgcatatccatagatattgaatgtctctgcattctattctatacctcttttcaaaaagatgctgttaaagacatggatagaaatagtagatgtagaaatagattgatttttctcctgcttactgttttacagcttgcttttcttttttcacctgacaatatgtcagggacttctttctacctcaggacataattttgtgccatttatttttcagctaacttcatttttaatatgaaacaactcaccatttaagccctaaaccaagacactgtaggtgtcttgaatagtaatttccaaacacctggcagtcactgcttgcatcagaatcaccaaattgctttaaaaagtacactatttaataaatcattgcattaaaaaagtaatgagagttggaattatagtggaattcttggattggctaccatccaggcttataaaacaaatactcctgtgtcccaatcatttggactagagaatctgtacttttattttttcgttttattttatttttttgagatggactctcactctgttgccaggctggagtgtagtggtgcgatcttggctcactgcaacctccgcctcccagcttcatgtgattctcctgcctcagcctcccgagtcgttttattattttaaaaaaaaattttttattgaggtataagtgacatagaatatttaaagtatacaatttgatatatttcagtgtttgtatacacctgtgaaaccatcaccataatcgacacagcaaatatatcatcatcctaaaaggtccctgctgcccctttgtgatacctccccccatccctcccctcaatcccactgatctgcttcctgtcattgtagcttagtttgcattttctagagcttgcatgagaacagtcatgcagtttatacttgtttttgtctggcttcttccactcagaatacttgttttgaaattttcctatgttgtgtatgtcagtagttcattcctttttatttctgaggatctgcatttttaaaaaatctgcctggtagattcttttgctccaccaggtttgggagcagacctgttcgggacatcctgatactttcatctccttcttcagttgccccaggaactctaacagtgctatagttctcctttccctggggctcgtctctaaggaactaggaagagcctggcctgaggctcctggtcctttatagtaactagaaggctgagagttaaatgtcagttcctcaggggcagagtttgttgtggcctaaaagaggggcatctggaatgcaaatagttcatgacgtgctgaacagcacatgcttaccacttaaggaatgcccccaaaccttcaaaaatcctcaaattcacaaagattgaggattttcgttcttggttcaggtctctgcttttctccttggtcacatttatgcttatagtcacttgttttcttcatataccctgtcactagaattcccctacatttttgaggatgcctaggacctcttacctaatggacattttcctaaaaggcccaatgtctgtcacctcatcagttattgcacccccagagatgatggagggtgactgagctggctggaggcagatcctgagctttcccaccagcttagtgactgccagcagcccacacacagtacacgccaggcctcagcaaagtacaaatggccaccagacctgggatgtcagaggcccttgggaatgttgaaaccaaggctgtcctaggccaactctattttatattacagacctgtgttgcttcacccttctgtgtcttgggcctccactgggcatggggtttgcagtagagacattggatcggctatcatctaggctactggtcttgttccagccctcttatccagcagcttgccagggtcaaaggctgcagggtgagggccagagcactgctctgtgccccatttgtgacctggtgactttagattctaactaccctggaatatacctccagaatatttgcaaagcccagatttgctgtacaaggcagcctgggcctttgctcttctacccgctgtaccatcttttgtcagtaaaatggttggttgctttgtgcagccattgtatcggatcttccctttggctttccctcctctgggctgttggccaggggctgccactggtgcaggctgcaggctccaagaggcagtgcaggaaaaggcttctgtggaagctgggcaggcctggatgccgggcaggctggggctagaattcaggtgtggcattagagggctcttatggtatgagtttggggttgaccttaaaggccattttgtttacagtccattcagaagtttactttttgtttaaataattaggattaacttgttaataaattccataccttgttctttagcgggattttaaatatagcatactcatctttccattctccttataaacctgatgaatttcggagctccccagagccaagacttgatattacttctcactttctcactggccctgcccagagcccaggtactctcacctgtttgaagaaggccatcccagtgtagttttccagtgttgttctgacctacctaaggtgctattaaaaatactggttcttgggcctgtctgagacctgtggagactctgaattcctagggctgtgaccctgggatctatgtttgagttaagtgccttagatgattacgatgtgctcttttttttttctttaaaggaaaaaacatacttttggtaggcttttaaagaacactgtaaaaaggatattcagtataatttttgttaaacatgtaatatgttttatgaaattttagaaaatacatgagtgaaagaaaaatgacttgtatgtctagcacccagcaatagccactattaatattttggtataatcttacagttttgtaatataatcatttccattacaaaagtcagatcatactacatctgcttttatgtaaattttcatttaaagaaatgccattatatttgttagatacaagttaagctgttgtattagagaccccaaaatacagctgctaaataaagagtttatttctttctcataataaagttcagagagaggtggcccagactgatgatagagggggttttggctgtgttccatgaggccatgcagggacccagttccctccatactatagctctgccatcctcggggtgttgtcctcttctgcctggttgcagcccggtcacctccatgtctcttccaactcacaggaaggcaggggagagggagctcagggcaagtgactttgtgcgaaggagatagcttagaagtgagcggtatggccatagcctggctgctcgcaggaggatgaggatgtggtttctatcttggcagccatgtacttagggaaagctttagggattctgttactgaaaggaagaaggcaagaatgggaatgggtggcagtctctgccataattaaaaatgcctttccgggtcagtaactaaagttttaaaatatttgtcataaagggcagtttctgttgcttgaattttattatcattttgttttttgcagtgatagttgttttgcagtcataaatcatctgtccttatatgttagagtgttttcttaggatgatacatttatagcagtagacttagtaagggagagggtaggcatagctttaagacttctaatacatattgtactttagaaaagttgaacccgtttacatttttattaccattcaagacctaaatataatttttaaatgtcattttaaaacggtttatcctaatactataggcatttcctcctgatcattatctacatatactctgtgatatgattatcacagaagtataaccttagtgtatatttggtttcatttttttccacttagtattatttatgagattatctgtaagccattttctgtttgtattttatttatatttatgacagtggcataccccttgtgtatatttatgtaacatatttacccaaatatctattatttgaaatggtaagttttggccacaaggtggcgcccggtaaaaagaaatgcccagacattctcttcaaaactgcttttggtttatgtgaagtctgttcttatcaactgcatacaattctactgtttgatgtaataaaacacagagcgagatgatatactacaagggattgttacttattaaaattgggctttagcttatttccaggtttttgttactggaaatcatcttaatacttttcttttgaaatatcctttcaagtggatagcttgaagtcctaagtgacttagcacaaagtcaaatttagaacttcagtctgagctataggcaagtttcacctttgtattctgtgctcacgctggtgcttcatatgaatgctgaaggacatgtgacaaagtgatgaactgctgtctgtgttcagtggcagaatagtttgcatcaatatattttcactgacaaagatggggaacagatgtaccaacaggtgctgtgaatccaaattttgttttgcttgttaaatatgccacaggtatttaatatcagtcatttgcaacggcaaaaacagatgccaaatatttctcctgtaacaatcccctatagtatatcatctcactctgtcttttattacatcaaacagtagaattgtatgcagttgattagaacagacttcacataaatcaaaagcagttttgacaaagaatgtctgagcatttctttttaccaggcgccaccttgtggccaaaacttaccatttcaaattcatttggcttgccaattttgtgtttaatttttgacatacatttaatttgagagtgtagaggaaatatggatttgcactattttgaattttaaaaataagtgacattttgctaatattttatattatactattttaaatatgaataaatgtaaatttcaatacaatttcaatttcatggctctagtatccctaactagttagaattgtgctggcatgcagtactacttggtgattatactgataccagtactcaagaaaaagtgtttaatcaataccatcattaaaatttgactaatataactagacaagtagaatgggtaaatagaaaatatacagtcatccctcaagtatctttggggactggctgcagacatcatccccaaaccaaaattcacagatactcaagtcccttatataaaatgatgtagtatttgcatatagcctatgcatatcctttcatatgctttaaatcatctctagattacttatgatgcttaatacaatgtaaacgctatgtaagtaggtgttacactgttttaaaatttgtattttttattgttttattatcttttattgtttttttccaaatattttccatctgcaaaatatcacttggctgaatccacagatacaaaactggatatggagggccaattgtatattataattattttatctatatatatccatctctctctctctctctctattatatatatatatatgtatatatatatgtagctacctatctgtctacctttatttcccaagtaaaatgactaactctttgaaaaagcttaggatgcaaatttcagcagaaaatgtcagcataaagttccgctagcaggagcaactgttgatctagtataattgacagcatatgtgcatagctgctttcttgtgtcaggtggtagctgcttaacaatcatacaatgagaaattgagtcattcagtaaagcaggacaaaaccatgccatctcatttaaaataatgccttggaaatgaagagtaaatctggatattggtataaagttgatttccgaggttgtggctagctccagcaacctatgatgttatctctaactttggtgtcagataactcttttccaaattatcttctttttaggctgttaaattccaaaagtttcaagcctttgactggggaagcagtgccaagaattattttcattacaaccaggtaaagtttttagtcttttcattaaagggggccctgaaaactcatcaagaaagccagcctggcctatcaggattctggccaggctcaggtgctgtgggaaatgtttgcaggagttgactagtgtttgttttccatcagtccattcagaacaatccaagctttgtagctggtgttgtggactgggcctcctctctttgtcctttccccagctctaaataagaatcatcactgttatgcattactcagagtaatcacgaacatagcctgtagagtcagacagtcctatgtctgttatctggtagctatggggccttggccagattacattgcttaagcctcttccctgcactaataaaattagcatttttgagcatgctaaatgcactgaagtgtgaggcactttgctctgttttttaaatttaattcccagagcatctctgtgatgtaaacgtgattatctctgacttaaacatgaggacagtgaggcttagagaatttctgtgacttgtcttacacccaaagggaagaagtaggagggcatgaccccaaactctttccagccactattgccttgagtgtactacctatggagtacaacaccttccttgccggggattaaacattcattttggtgcccagcctacggcagcagctccacagctagtggcgattataattagcattctctcatttggggttagatttctttttttgtgccggtaatggcaacttgaaaagatactcaaagagattttaaaaatttagtctgttagtcaaacttactagacaatgtttaatggagattgcgcttattgtgattttgaaaattaaaacaacaacaacaacgaggctttctctggttccttttcattgtagagttatcctcccacatacaatgtgaaggacatgcttgtgccgactgcagtctggagcgggggtcacgactggcttgcagatgtctacgacgtcaatatcttactgactcagatcaccaacttggtgttccatgagagcattccggaatgggagcatcttgacttcatttggggcctggatgccccttggaggctttataataaaattattaatctaatgaggaaatatcagtgaaagctggacttgagctgtgtaccaccaagtcaatgattatgtcatgtgaaaatgtgtttgcttcatttctgtaaaacacttgtttttctttcccaggtcttttgtttttttatatccaagaaaatgataactttgaagatgcccagttcactctagtttcaattagaaacatactagctattttttctttaattagggctggaataggaagccagtgtctcaaccatagtattgtctctttaagtcttttaaatatcactgatgtgtaaaaaggtcattatatccattctgtttttaaaatttaaaatatattgactttttgcccttcataggacaaagtaatatatgtgttggaattttaaaattgtgttgtcattggtaaatctgtcactgacttaagcgaggtataaaagtacgcagttttcatgtccttgccttaaagagctctctagtctaacggtcttgtagttagagatctaaatgacattttatcatgttttcctgcagcaggtgcatagtcaaatccagaaatatcacagctgtgccagtaataaggatgctaacaattaattttatcaaacctaactgtgacagctgtgatttgacacgttttaattgctcaggttaaatgaaatagttttccggcgtcttcaaaaacaaattgcactgataaaacaaaaacaaaagtatgttttaaatgctttgaagactgatacactcaaccatctatattcatgagctctcaatttcatggcaggccatagttctacttatctgagaagcaaatccctgtggagactataccactattttttctgagattaatgtactcttggagcccgctactgtcgttattgatcacatctgtgtgaagccaaagccccgtggttgcccatgagaagtgtccttgttcattttcacccaaatgaagtgtgaacgtgatgttttcggatgcaaactcagctcagggattcattttgtgtcttagttttatatgcatccttatttttaatacacctgcttcacgtccctatgttgggaagtccatatttgtctgcttttcttgcagcatcatttccttacaatactgtccggtggacaaaatgacaattgatatgtttttctgatataattactttagctgcactaacagtacaatgcttgttaatggttaatataggcagggcgaatactactttgtaacttttaaagtcttaaacttttcaataaaattgagtgagacttataggcccaaagaattgtgtgtatgttttttctcttttatttaacagtcatccggttcttccctttctctcttggcagtacttggaactcttctcaaggttaatgtgtttactccgggaaatgaagggctaccaagtaacgagtgatttttaaaaatcatttttttgtatgccacaagagggcactcttggctttgcttgcaaatcttcccacgctctcttccctcactactttttcttttcttttcttttcttttcttttcttttcttttcttttcttttcttttctttattattattatactttaagttttagggtacatgtgcacaatgtgcaggttagttacatatgtatacatgtgccatgctggtgtgctgcacccattaactcgtcatttagcattatctcctaaagctatccctcctacctccccccaccccacaacagtccccagagtgtgatgttccccttcctgtgtccatgtgttctcattgttcaattcccacctatgagtgagaatatgcggtgtttggtttttttttcttgggatagtttactgagattgatgatttccaatttcatccacgtccctacaaaggacatgaactcatcattttttatggctgcatagcattccatggtgtatatgtgccacattttcttaatccagtctatcattgttggacatttgggttggttccaagtctttgctattgtgaatagtgccgcaataaacatacgtgtgcatgtgtctttatagcagcatgatttatagtcctttgggtatatacccagtaatgggatggctgggtcaaatggtatttctagttctagatccctgaggaatcgccacactgacttccacaatggttgaactagtttacagtcccaccaacagtgtaaaagtgttcctatttctccacatcctctccagcacccgttgtttcctgactttgtaatgatttccattctaactggtgtgagatggtatctcattgtggttttgattttcattcctctgatggccagtgatggtgagcattttttcatgtgttttttggctgcataaatgccttcttttgagaagtgtctgttcatgtcctcactactttttctaagattttctccagcatctaccagggaaggtagagaactttgttgctttctgagccagttggaatttgatggccagccatggtagtattatttctgttcaggagtataattaattcttgcctagtggataacatctggaaaacgttctcaaggaatacctgcccctcaaatgtaaactgaaaataaaaatttgcctaataggtaaatgagtgatcagaacttcagatgtggttgggggatgggtggcaggaggaaaaggttttaactcaggcctcacacatatataggactgaaatttagactagtgacatccccaaataatgattattttgtgtttgtgggtattaaaggtgttaattttttaaatataaacattaaaaatacactgtaatgtgatttcttgtcattaatttttcaatgtattaagagattttcaaaagataagtcactctgatctgttgtgacactgaaaggcaactttgaatagtaattattctgatttttaaaaagttacccaatcagtatttcattatgttgcgctaggtgcagatttgaacttcaattccaaattaagcaagcaaaactctttgtcaaattgtcttagcattactgaaaataatacttcacattttgtgttgtgccttacggggtttcaaagccttttcataaagctttaatatttgcaataatccctttgacaggtgaggcgagcatttgtttctctacaggcaagggaatgcctttttaatggggatgattttatatgccttaaaagtttcagagaggttgatgagagtttcaacagtataagtgagtagaagcgcccacttaaatgcgacatgtaagtggatcagaatagaaagtgtttaaaaccaaatatgcaccagtatttggacta

SEQ ID NO: 2 - DNA, 72 bp, Homo sapiens
tctagagtggatgtatatacaacacattctcctgctggaacttctgtgcaaaacatgttacactggagccag

SEQ ID NO: 3 - DNA, 18 bp, Artificial Sequence
  Synthetic Construct
  modified_base (1)..(18): 2′-methoxyethoxy
ctacttggctccagtgta

SEQ ID NO: 4 - DNA, 18 bp, Artificial Sequence
  Synthetic Construct
  modified_base (1)..(18): 2′-methoxyethoxy
aatgcctacttggctcca

SEQ ID NO: 5 - DNA, 18 bp, Artificial Sequence
  Synthetic Construct
  modified_base (1)..(18): 2′-methoxyethoxy
cctggaatgcctacttgg

SEQ ID NO: 6 - DNA, 18 bp, Artificial Sequence
  Synthetic Construct
  modified_base (1)..(18): 2′-methoxyethoxy
gcactcctggaatgccta

SEQ ID NO: 7 - DNA, 18 bp, Artificial Sequence
  Synthetic Construct
  modified_base (1)..(18): 2′-methoxyethoxy
caaatgcactcctggaat

SEQ ID NO: 8 - DNA, 18 bp, Artificial Sequence
  Synthetic Construct
  modified_base (1)..(18): 2′-methoxyethoxy
aaccccaaatgcactcct

SEQ ID NO: 9 - DNA, 18 bp, Artificial Sequence
  Synthetic Construct
  modified_base (1)..(18): 2′-methoxyethoxy
acatgaaccccaaatgca

SEQ ID NO: 10 - DNA, 18 bp, Artificial Sequence
  Synthetic Construct
  modified_base (1)..(18): 2′-methoxyethoxy
attttacatgaaccccaa

SEQ ID NO: 11 - DNA, 18 bp, Artificial Sequence
  Synthetic Construct
  modified_base (1)..(18): 2′-methoxyethoxy
tgttgattttacatgaac

SEQ ID NO: 12 - DNA, 18 bp, Artificial Sequence
  Synthetic Construct
  modified_base (1)..(18): 2′-methoxyethoxy
tctgatgttgattttaca

SEQ ID NO: 13 - DNA, 18 bp, Artificial Sequence
  Synthetic Construct
  modified_base (1)..(18): 2′-methoxyethoxy
acctttctgatgttgatt

SEQ ID NO: 14 - DNA, 18 bp, Artificial Sequence
  Synthetic Construct
  modified_base (1)..(18): 2′-methoxyethoxy
cccagacctttctgatgt

SEQ ID NO: 15 - DNA, 16 bp, Artificial Sequence
  Synthetic Construct
  modified_base (1)..(16): 2′-methoxyethoxy
cagatttgtaagcagg

SEQ ID NO: 16 - DNA, 16 bp, Artificial Sequence
  Synthetic Construct
  modified_base (1)..(16): 2′-methoxyethoxy
tcaaagcactaaaaac

SEQ ID NO: 17 - DNA, 16 bp, Artificial Sequence
  Synthetic Construct
  modified_base (1)..(16): 2′-methoxyethoxy
aaccccaaatgcactc

SEQ ID NO: 18 - DNA, 16 bp, Artificial Sequence
  Synthetic Construct
  modified_base (1)..(16): 2′-methoxyethoxy
gcactcctggaatgcc

SEQ ID NO: 19 - DNA, 19 bp, Artificial Sequence
  Synthetic Construct
  modified_base (1)..(19): 2′-methoxyethoxy
atgcactcctggaatgcct

SEQ ID NO: 20 - DNA, 18 bp, Artificial Sequence
  Synthetic Construct
  modified_base (1)..(18): 2′-methoxyethoxy
gcccttcaaagcactaaa

SEQ ID NO: 21 - DNA, 18 bp, Artificial Sequence
  Synthetic Construct
  modified_base (1)..(18): 2′-methoxyethoxy
ttcaaagcactaaaaact

SEQ ID NO: 22 - DNA, 19 bp, Artificial Sequence
  Synthetic Construct
  modified_base (1)..(19): 2′-methoxyethoxy
accccaaatgcactcctgg

SEQ ID NO: 23 - DNA, 18 bp, Artificial Sequence
  Synthetic Construct
  modified_base (1)..(18): 2′-methoxyethoxy
cccaaatgcactcctgga

SEQ ID NO: 24 - DNA, 17 bp, Artificial Sequence
  Synthetic Construct
  modified_base (1)..(17): 2′-methoxyethoxy
caaatgcactcctggaa

SEQ ID NO: 25 - DNA, 16 bp, Artificial Sequence
  Synthetic Construct
  modified_base (1)..(16): 2′-methoxyethoxy
aatgcactcctggaat

SEQ ID NO: 26 - DNA, 16 bp, Artificial Sequence
  Synthetic Construct
  modified_base (1)..(16): 2′-methoxyethoxy
ccaaatgcactcctgg

SEQ ID NO: 27 - DNA, 17 bp, Artificial Sequence
  Synthetic Construct
  modified_base (1)..(17): 2′-methoxyethoxy
tgataataaacattgta

SEQ ID NO: 28 - DNA, 16 bp, Artificial Sequence
  Synthetic Construct
  modified_base (1)..(16): 2′-methoxyethoxy
cactcctggaatgcct

SEQ ID NO: 29 - DNA, 17 bp, Artificial Sequence
  Synthetic Construct
  modified_base (1)..(17): 2′-methoxyethoxy
atgcactcctggaatgc

SEQ ID NO: 30 - DNA, 20 bp, Artificial Sequence
  Synthetic Construct
  modified_base (1)..(20): 2′-methoxyethoxy
aaatgcactcctggaatgcc

SEQ ID NO: 31 - DNA, 16 bp, Artificial Sequence
  Synthetic Construct
  modified_base (1)..(16): 2′-methoxyethoxy
tgcccttcaaagcact

SEQ ID NO: 32 - DNA, 18 bp, Artificial Sequence
  Synthetic Construct
  modified_base (1)..(18): 2′-methoxyethoxy
aaatgcactcctggaatg

SEQ ID NO: 33 - DNA, 17 bp, Artificial Sequence
  Synthetic Construct
  modified_base (1)..(17): 2′-methoxyethoxy
accccaaatgcactcct

SEQ ID NO: 34 - DNA, 20 bp, Artificial Sequence
  Synthetic Construct
  modified_base (1)..(20): 2′-methoxyethoxy
ccccaaatgcactcctggaa

SEQ ID NO: 35 - DNA, 20 bp, Artificial Sequence
  Synthetic Construct
  modified_base (1)..(20): 2′-methoxyethoxy
cccaaatgcactcctggaat

SEQ ID NO: 36 - DNA, 19 bp, Artificial Sequence
  Synthetic Construct
  modified_base (1)..(19): 2′-methoxyethoxy
gtctttctatttggaaagg

SEQ ID NO: 37 - DNA, 18 bp, Artificial Sequence
  Synthetic Construct
  modified_base (1)..(18): 2′-methoxyethoxy
ttgtaagcaggttgtctt

SEQ ID NO: 38 - DNA, 18 bp, Artificial Sequence
  Synthetic Construct
  modified_base (1)..(18): 2′-methoxyethoxy
ccaaatgcactcctggaa

SEQ ID NO: 39 - DNA, 16 bp, Artificial Sequence
  Synthetic Construct
  modified_base (1)..(16): 2′-methoxyethoxy
ttctatttggaaaggg

SEQ ID NO: 40 - DNA, 20 bp, Artificial Sequence
  Synthetic Construct
  modified_base (1)..(20): 2′-methoxyethoxy
gtaagcaggttgtctttcta

SEQ ID NO: 41 - DNA, 18 bp, Artificial Sequence
  Synthetic Construct
  modified_base (1)..(18): 2′-methoxyethoxy
ccccaaatgcactcctgg

SEQ ID NO: 42 - DNA, 20 bp, Artificial Sequence
  Synthetic Construct
  modified_base (1)..(20): 2′-methoxyethoxy
caaatgcactcctggaatgc

SEQ ID NO: 43 - DNA, 19 bp, Artificial Sequence
  Synthetic Construct
  modified_base (1)..(19): 2′-methoxyethoxy
aatgcactcctggaatgcc

SEQ ID NO: 44 - DNA, 18 bp, Artificial Sequence
  Synthetic Construct
  modified_base (1)..(18): 2′-methoxyethoxy
tgcactcctggaatgcct

SEQ ID NO: 45 - DNA, 19 bp, Artificial Sequence
  Synthetic Construct
  modified_base (1)..(19): 2′-methoxyethoxy
aaccccaaatgcactcctg

SEQ ID NO: 46 - DNA, 20 bp, Artificial Sequence
  Synthetic Construct
  modified_base (1)..(20): 2′-methoxyethoxy
tttgtaagcaggttgtcttt

SEQ ID NO: 47 - DNA, 17 bp, Artificial Sequence
  Synthetic Construct
  modified_base (1)..(17): 2′-methoxyethoxy
aaatgcactcctggaat

SEQ ID NO: 48 - DNA, 17 bp, Artificial Sequence
  Synthetic Construct
  modified_base (1)..(17): 2′-methoxyethoxy
gcaggttgtctttctat

SEQ ID NO: 49 - DNA, 17 bp, Artificial Sequence
  Synthetic Construct
  modified_base (1)..(17): 2′-methoxyethoxy
aatgcactcctggaatg

SEQ ID NO: 50 - DNA, 16 bp, Artificial Sequence
  Synthetic Construct
  modified_base (1)..(16): 2′-methoxyethoxy
tgcactcctggaatgc

SEQ ID NO: 51 - DNA, 16 bp, Artificial Sequence
  Synthetic Construct
  modified_base (1)..(16): 2′-methoxyethoxy
cccaaatgcactcctg

SEQ ID NO: 52 - DNA, 17 bp, Artificial Sequence
  Synthetic Construct
  modified_base (1)..(17): 2′-methoxyethoxy
aaccccaaatgcactcc

SEQ ID NO: 53 - DNA, 16 bp, Artificial Sequence
  Synthetic Construct
  modified_base (1)..(16): 2′-methoxyethoxy
ccccaaatgcactcct

SEQ ID NO: 54 - DNA, 18 bp, Artificial Sequence
  Synthetic Construct
  modified_base (1)..(18): 2′-methoxyethoxy
tatttggaaagggtttgc

SEQ ID NO: 55 - DNA, 16 bp, Artificial Sequence
  Synthetic Construct
  modified_base (1)..(16): 2′-methoxyethoxy
agcactaaaaactaga

SEQ ID NO: 56 - DNA, 20 bp, Artificial Sequence
  Synthetic Construct
  modified_base (1)..(20): 2′-methoxyethoxy
aaccccaaatgcactcctgg

SEQ ID NO: 57 - DNA, 18 bp, Artificial Sequence
  Synthetic Construct
  modified_base (1)..(18): 2′-methoxyethoxy
accccaaatgcactcctg

SEQ ID NO: 58 - DNA, 16 bp, Artificial Sequence
  Synthetic Construct
  modified_base (1)..(16): 2′-methoxyethoxy
caaatgcactcctgga

SEQ ID NO: 59 - DNA, 19 bp, Artificial Sequence
  Synthetic Construct
  modified_base (1)..(19): 2′-methoxyethoxy
cccaaatgcactcctggaa

SEQ ID NO: 60 - DNA, 19 bp, Artificial Sequence
  Synthetic Construct
  modified_base (1)..(19): 2′-methoxyethoxy
gttgattttacatgaaccc

SEQ ID NO: 61 - DNA, 16 bp, Artificial Sequence
  Synthetic Construct
  modified_base (1)..(16): 2′-methoxyethoxy
actcctggaatgccta

SEQ ID NO: 62 - DNA, 17 bp, Artificial Sequence
  Synthetic Construct
  modified_base (1)..(17): 2′-methoxyethoxy
ttctgatgttgatttta

SEQ ID NO: 63 - DNA, 20 bp, Artificial Sequence
  Synthetic Construct
  modified_base (1)..(20): 2′-methoxyethoxy
ccaaatgcactcctggaatg

SEQ ID NO: 64 - DNA, 19 bp, Artificial Sequence
  Synthetic Construct
  modified_base (1)..(19): 2′-methoxyethoxy
aaatgcactcctggaatgc

SEQ ID NO: 65 - DNA, 18 bp, Artificial Sequence
  Synthetic Construct
  modified_base (1)..(18): 2′-methoxyethoxy
atgcactcctggaatgcc

SEQ ID NO: 66 - DNA, 17 bp, Artificial Sequence
  Synthetic Construct
  modified_base (1)..(17): 2′-methoxyethoxy
gcactcctggaatgcct

SEQ ID NO: 67 - DNA, 16 bp, Artificial Sequence
  Synthetic Construct
  modified_base (1)..(16): 2′-methoxyethoxy
accccaaatgcactcc

SEQ ID NO: 68 - DNA, 16 bp, Artificial Sequence
  Synthetic Construct
  modified_base (1)..(16): 2′-methoxyethoxy
ataataaacattgtat

SEQ ID NO: 69 - DNA, 19 bp, Artificial Sequence
  Synthetic Construct
  modified_base (1)..(19): 2′-methoxyethoxy
tgcactcctggaatgccta

SEQ ID NO: 70 - DNA, 16 bp, Artificial Sequence
  Synthetic Construct
  modified_base (1)..(16): 2′-methoxyethoxy
atgcactcctggaatg

SEQ ID NO: 71 - DNA, 19 bp, Artificial Sequence
  Synthetic Construct
  modified_base (1)..(19): 2′-methoxyethoxy
caaatgcactcctggaatg

SEQ ID NO: 72 - DNA, 17 bp, Artificial Sequence
  Synthetic Construct
  modified_base (1)..(17): 2′-methoxyethoxy
tgcactcctggaatgcc

SEQ ID NO: 73 - DNA, 20 bp, Artificial Sequence
  Synthetic Construct
  modified_base (1)..(20): 2′-methoxyethoxy
aatgcactcctggaatgcct

SEQ ID NO: 74 - DNA, 19 bp, Artificial Sequence
  Synthetic Construct
  modified_base (1)..(19): 2′-methoxyethoxy
tgttgattttacatgaacc

SEQ ID NO: 75 - DNA, 18 bp, Artificial Sequence
  Synthetic Construct
  modified_base (1)..(18): 2′-methoxyethoxy
tctatttggaaagggttt

SEQ ID NO: 76 - DNA, 19 bp, Artificial Sequence
  Synthetic Construct
  modified_base (1)..(19): 2′-methoxyethoxy
ccccaaatgcactcctgga

SEQ ID NO: 77 - DNA, 17 bp, Artificial Sequence
  Synthetic Construct
  modified_base (1)..(17): 2′-methoxyethoxy
ccccaaatgcactcctg

SEQ ID NO: 78 - DNA, 16 bp, Artificial Sequence
  Synthetic Construct
  modified_base (1)..(16): 2′-methoxyethoxy
tctgatgttgatttta

SEQ ID NO: 79 - DNA, 17 bp, Artificial Sequence
  Synthetic Construct
  modified_base (1)..(17): 2′-methoxyethoxy
ccaaatgcactcctgga

SEQ ID NO: 80 - DNA, 16 bp, Artificial Sequence
  Synthetic Construct
  modified_base (1)..(16): 2′-methoxyethoxy
aaatgcactcctggaa

SEQ ID NO: 81 - DNA, 18 bp, Artificial Sequence
  Synthetic Construct
  modified_base (1)..(18): 2′-methoxyethoxy
ataataaacattgtattt

SEQ ID NO: 82 - DNA, 17 bp, Artificial Sequence
  Synthetic Construct
  modified_base (1)..(17): 2′-methoxyethoxy
cactcctggaatgccta

SEQ ID NO: 83 - DNA, 18 bp, Artificial Sequence
  Synthetic Construct
  modified_base (1)..(18): 2′-methoxyethoxy
aatgcactcctggaatgc

SEQ ID NO: 84 - DNA, 17 bp, Artificial Sequence
  Synthetic Construct
  modified_base (1)..(17): 2′-methoxyethoxy
cccaaatgcactcctgg

SEQ ID NO: 85 - DNA, 20 bp, Artificial Sequence
  Synthetic Construct
  modified_base (1)..(20): 2′-methoxyethoxy
accccaaatgcactcctgga

SEQ ID NO: 86 - DNA, 20 bp, Artificial Sequence
  Synthetic Construct
  modified_base (1)..(20): 2′-methoxyethoxy
tctatttggaaagggtttgc

SEQ ID NO: 87 - DNA, 20 bp, Artificial Sequence
  Synthetic Construct
  modified_base (1)..(20): 2′-methoxyethoxy
atgcactcctggaatgccta

SEQ ID NO: 88 - DNA, 18 bp, Artificial Sequence
  Synthetic Construct
  modified_base (1)..(18): 2′-methoxyethoxy
atgttgattttacatgaa

SEQ ID NO: 89 - DNA, 19 bp, Artificial Sequence
  Synthetic Construct
  modified_base (1)..(19): 2′-methoxyethoxy
ccaaatgcactcctggaat

SEQ ID NO: 90 - DNA, 18 bp, Artificial Sequence
  Synthetic Construct
  modified_base (1)..(18): 2′-methoxyethoxy
taataaacattgtatttt

SEQ ID NO: 91 - DNA, 18 bp, Artificial Sequence
  Synthetic Construct
  modified_base (1)..(18): 2′-methoxyethoxy
ttgataataaacattgta

SEQ ID NO: 92 - DNA, 25 bp, Artificial Sequence
  Synthetic Construct
ttcttctgtgtggatttaatgagag

SEQ ID NO: 93 - DNA, 20 bp, Artificial Sequence
  Synthetic Construct
aattcttggcactgcttccc

SEQ ID NO: 94 - DNA, 18 bp, Artificial Sequence
  Synthetic Construct
gaaaacagcattaaggtg

SEQ ID NO: 95 - DNA, 18 bp, Artificial Sequence
  Synthetic Construct
agaatgaaaacagcatta

SEQ ID NO: 96 - DNA, 18 bp, Artificial Sequence
  Synthetic Construct
aatgaagaatgaaaacag

SEQ ID NO: 97 - DNA, 18 bp, Artificial Sequence
  Synthetic Construct
attgaaatgaagaatgaa

SEQ ID NO: 98 - DNA, 18 bp, Artificial Sequence
  Synthetic Construct
aatacattgaaatgaaga

SEQ ID NO: 99 - DNA, 18 bp, Artificial Sequence
  Synthetic Construct
aaataaatacattgaaat

SEQ ID NO: 100 - DNA, 18 bp, Artificial Sequence
  Synthetic Construct
ctgcaaaataaatacatt

SEQ ID NO: 101 - DNA, 18 bp, Artificial Sequence
  Synthetic Construct
ctagactgcaaaataaat

SEQ ID NO: 102 - DNA, 18 bp, Artificial Sequence
  Synthetic Construct
ccactctagactgcaaaa

SEQ ID NO: 103 - DNA, 18 bp, Artificial Sequence
  Synthetic Construct
tacatccactctagactg

SEQ ID NO: 104 - DNA, 18 bp, Artificial Sequence
  Synthetic Construct
gtatatacatccactcta

SEQ ID NO: 105 - DNA, 18 bp, Artificial Sequence
  Synthetic Construct
gtgttgtatatacatcca

SEQ ID NO: 106 - DNA, 18 bp, Artificial Sequence
  Synthetic Construct
agaatgtgttgtatatac

SEQ ID NO: 107 - DNA, 18 bp, Artificial Sequence
  Synthetic Construct
gcaggagaatgtgttgta

SEQ ID NO: 108 - DNA, 18 bp, Artificial Sequence
  Synthetic Construct
ttccagcaggagaatgtg

SEQ ID NO: 109 - DNA, 18 bp, Artificial Sequence
  Synthetic Construct
agaagttccagcaggaga

SEQ ID NO: 110 - DNA, 18 bp, Artificial Sequence
  Synthetic Construct
ttgcacagaagttccagc

SEQ ID NO: 111 - DNA, 18 bp, Artificial Sequence
  Synthetic Construct
atgttttgcacagaagtt

SEQ ID NO: 112 - DNA, 18 bp, Artificial Sequence
  Synthetic Construct
gtaacatgttttgcacag

SEQ ID NO: 113 - DNA, 18 bp, Artificial Sequence
  Synthetic Construct
ccagtgtaacatgttttg

SEQ ID NO: 114 - DNA, 18 bp, Artificial Sequence
  Synthetic Construct
tggctccagtgtaacatg

SEQ ID NO: 115 - DNA, 18 bp, Artificial Sequence
  Synthetic Construct
taaggtggcattgataat

SEQ ID NO: 116 - DNA, 18 bp, Artificial Sequence
  Synthetic Construct
cagcattaaggtggcatt

(19) United States
(12) Patent Application Publication
(10) Pub. No.: US 2022/0228152 A1
(43) Pub. Date: Jul. 21, 2022

(54) OLIGONUCLEOTIDE THERAPY FOR WOLMAN DISEASE AND CHOLESTERYL ESTER STORAGE DISEASE

(71) Applicant: Deep Genomics Incorporated, Toronto (CA)

(72) Inventors: Daniele MERICO, Toronto (CA); Kahlin CHEUNG-ONG, Toronto (CA); Mark SUN, Toronto (CA)

(21) Appl. No.: 17/615,413

(22) PCT Filed: May 29, 2020
(86) PCT No.: PCT/CA2020/050740

§ 371 Date: Nov. 30, 2021

Related U.S. Application Data
(60) Provisional application No. 62/854,719, filed on May 30, 2019.

(51) Int. Cl. C12N 15/113

(52) U.S. Cl. CPC C12N 15/1137; C12Y 301/01013; C12N 2320/33; C12N 2310/11; C12N 2310/322; C12N 2310/3231; C12N 2310/3233; C12N 2310/315

(57) ABSTRACT

The present disclosure provides antisense oligonucleotides, compositions, and methods that target a LIPA intron flanking exon 8, thereby modulating splicing of LIPA pre-mRNA to increase the level of LIPA mRNA molecules having exon 8, e.g., to provide a therapy for Wolman Disease or Cholesteryl Ester Storage Disease. The present disclosure provides an antisense oligonucleotide including a nucleobase sequence at least 70% complementary to a LIPA pre-mRNA target sequence in a 5′-flanking intron, a 3′-flanking intron, or a combination of exon 8 and the 5′-flanking or 3′-flanking intron.

### FIELD OF THE INVENTION

The present invention relates to the field of oligonucleotides and their use for the treatment of disease. In particular, the invention pertains to antisense oligonucleotides that may be used in the treatment of Wolman Disease and Cholesteryl Ester Storage Disease.

### BACKGROUND

Lysosomal acid lipase (LAL) or LIPA (lipase A, lysosomal acid type) (Entrez Gene ID 3988) is a lysosomal enzyme encoded by the LIPA gene required for the hydrolysis of cholesteryl esters and triglycerides, which are derived from the internalization of plasma lipoprotein particles (chylomicron remnants, LDL, IDL) by endocytosis.

LIPA is expressed at medium to high levels in the spleen, brain, adipose tissue, and lung, whereas liver expression is in the medium to low range. Two curated NCBI Reference Sequence (RefSeq) isoforms are known: NM_000235 and NM_001127605; NM_000235 (chr10:90973326-91011796) being treated as the principal transcript.

Pathogenic loss of function variants in LIPA follow a recessive mode of inheritance. Complete loss-of-function (detectable LIPA activity 0-2.5%) results in the more severe Wolman Disease (WD), whereas partial loss-of-function (detectable LIPA activity 2-12%) results in the less severe Cholesteryl Ester Storage Disease (CESD). One such pathogenic loss of function LIPA variant is [Hg19/b37] chr10:90982268:C:T NM_000235.3(LIPA):c.894G>A (p.GIn298=).

CESD is characterized by LDL hypercholesterolemia, hypertriglyceridemia, HDL deficiency, atherosclerosis, hepatosplenomegaly with liver dysfunction, and abnormal intracellular lipid accumulation in many organs. Reported CESD patient deaths are mainly due to atherosclerotic vascular disease or hepatic disease.

Accumulated lipids mainly consist of cholesteryl ester, and only secondarily triglycerides. Accumulation mainly occurs in liver hepatocytes, macrophages (in the liver, spleen, lymph nodes and other organs), adrenal glands and intestine, and is in general proportional to the tissue and cell type contribution to LDL absorption and metabolism.

CESD liver is almost always characterized by hepatomegaly and steatosis, which progresses to fibrosis in the majority of patients and eventually to micronodular cirrhosis and liver failure in about 10-15% of CESD cases. In the liver, lipid deposition is observed in both hepatocytes and Kupffer cells.

Excess LDL cholesterol causes accelerated atherosclerosis, resulting in increased risk of coronary artery disease and stroke. Elevated blood LDL is caused by low intracellular cholesterol in hepatocytes, stimulating intracellular cholesterol synthesis and VLDL production, which results in increased LDL. A similar mechanism leads to reduced HDL production: low intracellular cholesterol reduces the ABCA1 transporter expression, which is required for HDL production. Low HDL is expected to further exacerbate the atherosclerotic process due to excess LDL and its deposition on artery walls.

Splenomegaly is often present in patients with CESD and WD and can cause secondary complications such as anemia and/or thrombocytopenia (resulting in bleeding episodes). Splenomegaly is secondary to liver disease, primarily because of portal vein hypertension, although macrophage cholesterol accumulation and foam cell formation has also been reported in the spleen; this may be secondary to LDL cholesterol excess and spleen disease. Thrombocytopenia is secondary to splenomegaly and liver disease.

Gastrointestinal lipid deposition is present in the majority of patients, resulting in malabsorption, abdominal pain and diarrhea, but more severe gastrointestinal pathology can also be present. Esophageal varices are typically present in patients with more severe liver dysfunction.

CESD onset occurs in infancy to adulthood. Whereas untreated WD is fatal within the first 6 months of life, individuals with CESD may have a normal or near normal lifespan. Enzyme activity level appears to not be strictly predictive of phenotype and disease course.

Current WD treatment is enzyme replacement with a recombinant form of LIPA, delivered by intravenous injection bi-weekly. Similarly, enzyme replacement can also ameliorate the CESD disease phenotype. Prior to the introduction of this treatment, WD could only be treated by hematopoietic stem cell transplantation, which had a mixed success rate. Enzyme replacement is complicated by patient immune response to the enzyme and short blood half-life.

Statins (and/or other related drugs) and a low-fat diet are somewhat effective at normalizing LDL cholesterol levels and preventing accelerated atherosclerosis in CESD. Statins are not effective, however, at preventing liver disorder.

In certain cases, CESD may also be treated by liver transplant, but this is expensive, it requires organ availability, the transplant survival rate is <100%, and recipients need to receive immunosuppressive therapy.

In general, greater than 15-20% normal LIPA activity is expected to be sufficient for a robust therapeutic intervention, since pathological levels of LIPA are typically <15%.

Certain human genetic diseases (e.g., caused by genetic aberrations, such as point mutations) may be caused by aberrant splicing. As such, there is a need for a splicing modulator to treat diseases that are caused by aberrant splicing.

### SUMMARY OF THE INVENTION

In general, the invention provides antisense oligonucleotides and methods of their use in the treatment of conditions associated with incorrect splicing of LIPA pre-mRNA (e.g., exon 8 skipping).

In one aspect, the invention provides an antisense oligonucleotide including a nucleobase sequence that is at least 70% (e.g., at least 80%, at least 90%, at least 95%, or 100%) complementary to a LIPA pre-mRNA target sequence (e.g., g.34393G>A mutation in SEQ ID NO: 1). The LIPA pre-mRNA target sequence may be disposed in, e.g., a 5′-flanking intron, a 3′-flanking intron, or a combination of an exon and the 5′-flanking or 3′-flanking intron. In some embodiments, the oligonucleotide includes a targeting moiety covalently linked to the nucleobase sequence. In some embodiments, the antisense oligonucleotide includes a total of 15 to 22 (e.g., 16, 17, 18, or 19) nucleosides in the nucleobase sequence. In some embodiments, the antisense oligonucleotide includes a total of 20 to 30 (e.g., 20, 21, or 22) nucleosides in the nucleobase sequence.

In some embodiments, the LIPA target sequence is in a 5′-flanking intron adjacent to exon 8, 3′-flanking intron adjacent to exon 8, or a combination of exon 8 and the adjacent 5′-flanking or 3′-flanking intron. In certain embodiments, the LIPA target sequence reduces the binding of a splicing factor to an intronic splicing silencer in the 5′-flanking or 3′-flanking intron.

In particular embodiments, the LIPA target sequence includes at least one nucleotide (e.g., 5, 10, 11, 12, 13, 14, 15, 16, 17, 18, 19, or 20 consecutive nucleotides) located among positions 34222-34321 in SEQ ID NO: 1 (e.g., the LIPA target sequence is wholly within these positions). In further embodiments, the LIPA target sequence includes at least one nucleotide (e.g., 5, 10, 11, 12, 13, 14, 15, 16, 17, 18, 19, or 20 consecutive nucleotides) located among positions 34394-34493 in SEQ ID NO: 1. In yet further embodiments, the LIPA target sequence includes at least one nucleotide (e.g., 5, 10, 11, 12, 13, 14, 15, 16, 17, 18, 19, or 20 consecutive nucleotides) located among positions 34398-34480 in SEQ ID NO: 1 (e.g., the LIPA target sequence is wholly within these positions). In still further embodiments, the LIPA target sequence includes at least one nucleotide (e.g., 5, 10, 11, 12, 13, 14, 15, 16, 17, 18, 19, or 20 consecutive nucleotides) located among positions 34401-34422 in SEQ ID NO: 1 (e.g., the LIPA target sequence is wholly within these positions). In other embodiments, the LIPA target sequence includes at least one nucleotide (e.g., 5, 10, 11, 12, 13, 14, 15, 16, 17, 18, 19, or 20 consecutive nucleotides) located among positions 34456-34473 in SEQ ID NO: 1 (e.g., the LIPA target sequence is wholly within these positions).

In yet other embodiments, the nucleobase sequence is complementary to a sequence within the 5′-flanking intron of the pre-mRNA. In still other embodiments, the LIPA target sequence is located within the 5′-flanking intron among positions up to 34321 in SEQ ID NO: 1. In some embodiments, the nucleobase sequence has at least 70% (e.g., at least 80%, at least 90%, at least 95%, or 100%) sequence identity to SEQ ID NO: 68, 81, or 98. In some embodiments, the nucleobase sequence is complementary to an aberrant LIPA sequence having a mutation in SEQ ID NO: 1 (e.g., a g.34393G>A mutation in SEQ ID NO: 1).

In certain embodiments, the LIPA target sequence is located within the 3′-flanking intron. In particular embodiments, the LIPA target sequence is located within the 3′-flanking intron among positions up to 34500 in SEQ ID NO: 1.

In further embodiments, the nucleobase sequence has at least 70% (e.g., at least 80%, at least 90%, at least 95%, or 100%) sequence identity to any one of SEQ ID NOs: 7, 9-15, 22-26, 29, 32, 34-41, 45-49, 51, 54, 56-60, 62-64, 67, 70-72, 74-80, 83-86, 88 and 89. In yet further embodiments, the LIPA target sequence is located among positions 34394 to 34498 in SEQ ID NO: 1. In still further embodiments, the nucleobase sequence has at least 70% (e.g., at least 80%, at least 90%, at least 95%, or 100%) sequence identity to SEQ ID NO: 7, 9, 12, 13, 15, 22, 23, 24, 25, 26, 32, 34, 35, 36, 38, 39, 40, 41, 45, 47, 48, 49, 51, 54, 56, 57, 58, 59, 62, 63, 64, 70, 71, 74, 75, 76, 77, 78, 79, 80, 83, 84, 85, 86, 88, or 89. In some embodiments, the nucleobase sequence has at least 70% (e.g., at least 80%, at least 90%, at least 95%, or 100%) sequence identity to SEQ ID NO: SEQ ID NO: 7, 22, 23, 24, 26, 32, 34, 38, 41, 49, 56, 58, 59, 63, 70, 71, 75, 76, 79, 80, 84, 85, 86, or 88.

In other embodiments, the nucleobase sequence has at least 70% (e.g., at least 80%, at least 90%, at least 95%, or 100%) sequence identity to SEQ ID NO: 84. In yet other embodiments, the nucleobase sequence has at least 70% (e.g., at least 80%, at least 90%, at least 95%, or 100%) sequence identity to SEQ ID NO: 26. In still other embodiments, the nucleobase sequence has at least 70% (e.g., at least 80%, at least 90%, at least 95%, or 100%) sequence identity to SEQ ID NO: 22. In some embodiments, the nucleobase sequence has at least 70% (e.g., at least 80%, at least 90%, at least 95%, or 100%) sequence identity to SEQ ID NO: 85. In certain embodiments, the nucleobase sequence has at least 70% (e.g., at least 80%, at least 90%, at least 95%, or 100%) sequence identity to SEQ ID NO: 76. In particular embodiments, the nucleobase sequence has at least 70% (e.g., at least 80%, at least 90%, at least 95%, or 100%) sequence identity to SEQ ID NO: 41. In further embodiments, the nucleobase sequence has at least 70% (e.g., at least 80%, at least 90%, at least 95%, or 100%) sequence identity to SEQ ID NO: 56. In yet further embodiments, the nucleobase sequence has at least 70% (e.g., at least 80%, at least 90%, at least 95%, or 100%) sequence identity to SEQ ID NO: 23. In still further embodiments, the nucleobase sequence has at least 70% (e.g., at least 80%, at least 90%, at least 95%, or 100%) sequence identity to SEQ ID NO: 79. In some embodiments, the nucleobase sequence has at least 70% (e.g., at least 80%, at least 90%, at least 95%, or 100%) sequence identity to SEQ ID NO: 59. In certain embodiments, the nucleobase sequence has at least 70% (e.g., at least 80%, at least 90%, at least 95%, or 100%) sequence identity to SEQ ID NO: 58. In particular embodiments, the nucleobase sequence has at least 70% (e.g., at least 80%, at least 90%, at least 95%, or 100%) sequence identity to SEQ ID NO: 34. In further embodiments, the nucleobase sequence has at least 70% (e.g., at least 80%, at least 90%, at least 95%, or 100%) sequence identity to SEQ ID NO: 54.

In yet further embodiments, the 3′-terminal nucleotide of the oligonucleotide is complementary to the LIPA pre-mRNA corresponding to any one position selected from the group consisting of 34394-34398 SEQ ID NO: 1.

In still further embodiments, the nucleobase sequence has at least 70% (e.g., at least 80%, at least 90%, at least 95%, or 100%) sequence identity to any one of SEQ ID NOs: 7, 22, 23, 24, 26, 32, 34, 38, 41, 49, 56, 58, 59, 63, 70, 71, 75, 76, 79, 80, 84, 85, 86, and 88. In other embodiments, the sequence identity is at least 80% (e.g., at least 90%, at least 95% (e.g., 100%)).

In yet other embodiments, the antisense oligonucleotide includes at least one modified nucleobase. In still other embodiments, the antisense oligonucleotide includes at least one modified internucleoside linkage. In some embodiments, the modified internucleoside linkage is a phosphorothioate linkage. In certain embodiments, the phosphorothioate linkage is a stereochemically enriched phosphorothioate linkage. In particular embodiments, at least 50% of internucleoside linkages in the antisense oligonucleotide are independently the modified internucleoside linkage. In further embodiments, at least 70% (e.g., at least 80%, at least 90%, at least 95%, or 100%) of internucleoside linkages in the antisense oligonucleotide are independently the modified internucleoside linkage. In yet further embodiments, all internucleoside linkages in the antisense oligonucleotide are independently the modified internucleoside linkage.

In still further embodiments, the antisense oligonucleotide includes at least one modified sugar nucleoside. In some embodiments, at least one modified sugar nucleoside is a 2′-modified sugar nucleoside. In certain embodiments, at least one 2′-modified sugar nucleoside includes a 2′-modification selected from the group consisting of 2′-fluoro, 2′-methoxy, and 2′-methoxyethoxy. In particular embodiments, the 2′-modified sugar nucleoside includes the 2′-methoxyethoxy modification. In further embodiments, at least one modified sugar nucleoside is a bridged nucleic acid. In yet further embodiments, the bridged nucleic acid is a locked nucleic acid (LNA), ethylene-bridged nucleic acid (ENA), or cEt nucleic acid. In still further embodiments, all nucleosides in the antisense oligonucleotide are independently the modified sugar nucleosides. In some embodiments, the antisense oligonucleotide is a morpholino oligomer.

In certain embodiments, at least 50% (e.g., at least 70% or 100%) of the internucleoside linkages are phosphorothioate linkages, and at least 50% (e.g., at least 70% or 100%) of the sugars are 2′-modified, e.g., with 2′-methoxyethoxy. For example, the antisense oligonucleotide may be fully modified with phosphorothioate internucleoside linkaages and 2′-methoxyethoxy groups.

In certain embodiments, the antisense oligonucleotide, e.g., as modified as discussed herein, further includes a targeting moiety. In particular embodiments, the targeting moiety is covalently conjugated at the 5′-terminus of the antisense oligonucleotide. In further embodiments, the targeting moiety is covalently conjugated at the 3′-terminus of the antisense oligonucleotide. In yet further embodiments, the targeting moiety is covalently conjugated at an internucleoside linkage of the antisense oligonucleotide. In still further embodiments, the targeting moiety is covalently conjugated through a linker (e.g., a cleavable linker). In other embodiments, the linker is a cleavable linker. In yet other embodiments, the targeting moiety includes N-acetylgalactosamine (e.g., is an N-acetylgalactosamine cluster).

In some embodiments, the N-acetylgalactosamine cluster is of the following structure:

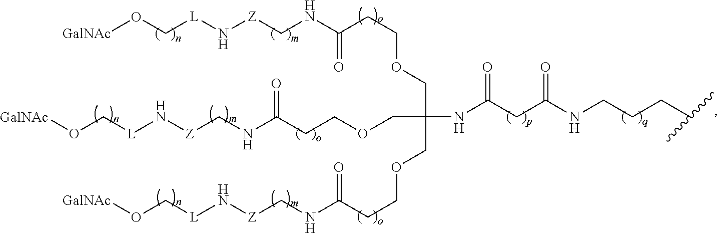

where each L is independently CO or CH₂, each Z is independently CO or CH₂, each n is independently 1 to 9, each m is independently 1 to 5, each o is independently 0 to 1, each p is independently 1 to 10, and each q is independently 1 to 10.

In certain embodiments, each L is CH₂. In particular embodiments, each Z is CO. In further embodiments, each n is 5. In yet further embodiments, each m is 2. In still further embodiments, each o is 1. In some embodiments, each p is 2. In particular embodiments, each p is 3. In other embodiments, each q is 4. In yet other embodiments, the N-acetylgalactosamine cluster is of the following structure:

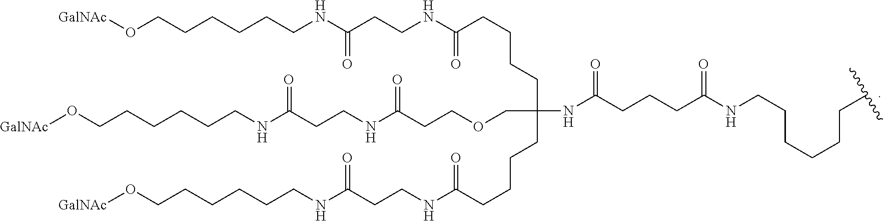

In still other embodiments, the antisense oligonucleotide includes at least 12 nucleosides. In some embodiments, the antisense oligonucleotide includes at least 16 nucleosides. In certain embodiments, the antisense oligonucleotide includes a total of 50 nucleosides or fewer (e.g., 30 nucleosides or fewer, or 20 nucleosides or fewer). In particular embodiments, the antisense oligonucleotide includes a total of 16 to 20 nucleosides.

In another aspect, the invention provides a pharmaceutical composition including the antisense oligonucleotide of the invention and a pharmaceutically acceptable excipient.

In yet another aspect, the invention provides a method of increasing the level of exon-containing (e.g., exon 8-containing) LIPA mRNA molecules in a cell expressing an aberrant LIPA gene. The method includes contacting the cell with the antisense oligonucleotide of the invention.

In some embodiments, the cell is in a subject. In certain embodiments, the cell is a hepatocyte. In particular embodiments, the cell is a Kupffer cell.

In still another aspect, the invention provides a method of treating Wolman Disease or Cholesteryl Ester Storage Disease in a subject having an aberrant LIPA gene. The method includes administering a therapeutically effective amount of the antisense oligonucleotide of the invention or the pharmaceutical composition of the invention to the subject in need thereof.

In some embodiments, the administering step is performed parenterally. In certain embodiments, the method further includes administering to the subject a therapeutically effective amount of a second therapy for Wolman Disease or Cholesteryl Ester Storage Disease. In particular embodiments, the second therapy is a recombinant lysosomal acid lipase or a statin or a salt thereof. In further embodiments, the second therapy is a hematopoietic stem cell transplantation.

In yet further embodiments, the aberrant LIPA gene is LIPA having a g.34393G>A mutation in SEQ ID NO: 1.

Recognized herein is the need for compositions and methods for treating diseases that may be caused by abnormal splicing resulting from an underlying genetic aberration. In some cases, antisense nucleic acid molecules, such as oligonucleotides, may be used to effectively modulate the splicing of targeted genes in genetic diseases, in order to alter the gene products produced. This approach can be applied in therapeutics to selectively modulate the expression and gene product composition for genes involved in genetic diseases.

The present disclosure provides compositions and methods that may advantageously use antisense oligonucleotides targeted to and hybridizable with nucleic acid molecules that encode for LIPA. Such antisense oligonucleotides may target one or more splicing regulatory elements in one or more exons (e.g., exon 8) or introns (e.g., 5′-flanking intro or 3′ flanking intron) of LIPA. These splicing regulatory elements modulate splicing of LIPA ribonucleic acid (RNA).

In one aspect, the present disclosure provides a LIPA RNA splice-modulating antisense oligonucleotide having a sequence targeted to an intron adjacent to an exon (e.g., exon 8) of LIPA. In some embodiments, a genetic aberration of LIPA includes the c.894G>A mutation. In some embodiments, the c.894G>A mutation results from LIPA chr10:90982268:C:T [hg19/b37] (g.34393G>A in SEQ ID NO: 1). In some embodiments, the antisense oligonucleotide has a sequence targeted to one or more splicing regulatory elements. In some embodiments, the one or more splicing regulatory elements include an intronic splicing silencer element. In some embodiments, the sequence is targeted to an intron adjacent to an abnormally spliced exon (e.g., a flanking intron). In some embodiments, the antisense oligonucleotide modulates variant splicing to yield an increase in exon inclusion (e.g., exon 8 inclusion). In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides.

In another aspect, the present disclosure provides a method for modulating splicing of LIPA RNA in a cell, tissue, or organ of a subject, including bringing the cell, tissue, or organ in contact with an antisense oligonucleotide including one or more sequences targeted to an intron adjacent to an exon (e.g., exon 8) of LIPA. In some embodiments, the genetic aberration of LIPA includes c.894G>A. In some embodiments, the c.894G>A results from LIPA chr10:90982268:C:T [hg19/b37] (g.34393G>A in SEQ ID NO: 1). In some embodiments, the antisense oligonucleotide has a sequence targeted to one or more splicing regulatory elements. In some embodiments, the one or more splicing regulatory elements are an intronic splicing silencer element. In some embodiments, the sequence is targeted to an intron adjacent to an abnormally spliced exon (e.g., a flanking intron). In some embodiments, the antisense oligonucleotide modulates variant splicing to yield an increase in exon inclusion (e.g., exon 8 inclusion), e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%, as compared to the ratio of exon-including LIPA transcripts (e.g., exon 8-including LIPA transcripts) to the total number of LIPA transcript molecules in a cell including LIPA gene having an exon-skipping mutation (e.g., an exon 8-skipping mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject has or is suspected of having a disease, e.g., Wolman Disease or Cholesteryl Ester Storage Disease, and the subject is monitored for a progression or regression of the disease in response to bringing the cell, tissue, or organ in contact with the composition.

In another aspect, the present disclosure provides a method for treating Wolman Disease or Cholesteryl Ester Storage Disease in a subject, including administering to the subject a therapeutically effective amount of an antisense oligonucleotide including a sequence targeted to an intron adjacent to an exon (e.g., exon 8) of LIPA. The antisense oligonucleotide modulates splicing of LIPA RNA. In some embodiments, the genetic aberration of LIPA includes the c.894G>A mutation. In some embodiments, the c.894G>A mutation results from LIPA chr10:90982268:C:T [hg19/b37] (g.34393G>A mutant of SEQ ID NO: 1). In some embodiments, the antisense oligonucleotide has a sequence targeted to one or more splicing regulatory elements. In some embodiments, the one or more splicing regulatory elements are an intronic splicing silencer element. In some embodiments, the sequence is targeted to an intron adjacent to an abnormally spliced exon of the genetic aberration of LIPA that modulates variant splicing of LIPA RNA (e.g., a flanking intron). In some embodiments, the antisense oligonucleotide modulates splicing to yield an increase in exon (e.g., exon 8) inclusion (e.g., increase by at least 5%, at least 10%, at least 15%, at least 20%, at least 25%, at least 30%, at least 35%, at least 40%, at least 45%, or at least 50%; e.g., up to 100%, up to 90%, up to 80%, up to 70%, up to 60%, up to 50%, up to 40%, up to 30%, up to 20%), as compared to the ratio of exon-including LIPA transcripts (e.g., exon 8-including LIPA transcripts) to the total number of LIPA transcript molecules in a cell including LIPA gene having an exon-skipping mutation (e.g., an exon 8-skipping mutation) in the absence of a treatment with an antisense oligonucleotide. In some embodiments, the antisense oligonucleotide has a length of 12 to 20 nucleotides. In some embodiments, the antisense oligonucleotide has a length of 12 to 30 nucleotides. In some embodiments, the antisense oligonucleotide has a length of 12 to 50 nucleotides. In some embodiments, the subject is monitored for a progression or regression of Wolman Disease or Cholesteryl Ester Storage Disease in response to administering to the subject the therapeutically effective amount of the antisense oligonucleotide.

In another aspect, the present disclosure provides a pharmaceutical composition for treatment of Wolman Disease or Cholesteryl Ester Storage Disease including an antisense oligonucleotide and a pharmaceutically acceptable carrier. The antisense oligonucleotide includes a sequence targeted to an intron adjacent to the abnormally spliced exon. The antisense oligonucleotide modulates splicing of LIPA RNA. In some embodiments, the genetic aberration of LIPA includes c.894G>A. In certain embodiments, the therapeutically effective amount is about 1 mg/kg to 10 mg/kg (e.g., about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg). In particular embodiments, the antisense oligonucleotide or the pharmaceutical composition is administered from once monthly to once weekly. In certain embodiments, the antisense oligonucleotide or the pharmaceutical composition is administered once weekly, biweekly, or monthly. In some embodiments, the c.894G>A mutation results from LIPA chr10:90982268:C:T [hg19/b37] (g.34393G>A mutant of SEQ ID NO: 1).

### Definitions

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description.

The term “about,” as used herein, represents ±10% of the specified value.

The term “acyl,” as used herein, represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, represents a chemical substituent of formula —OR, where R is acyl. An optionally substituted acyloxy is an acyloxy that is optionally substituted as described herein for acyl.

The term “alkane-tetrayl,” as used herein, represents a tetravalent, acyclic, straight or branched chain, saturated hydrocarbon group having from 1 to 16 carbons, unless otherwise specified. Alkane-tetrayl may be optionally substituted as described for alkyl.

The term “alkane-triyl,” as used herein, represents a trivalent, acyclic, straight or branched chain, saturated hydrocarbon group having from 1 to 16 carbons, unless otherwise specified. Alkane-triyl may be optionally substituted as described for alkyl.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, a substituted alkyl includes two substituents (oxo and hydroxy, or oxo and alkoxy) to form a group —L—CO—R, where L is a bond or optionally substituted C₁₋₁₁ alkylene, and R is hydroxyl or alkoxy. Each of the substituents may itself be unsubstituted or, valency permitting, substituted with unsubstituted substituent(s) defined herein for each respective group.

The term “alkylene,” as used herein, represents a divalent substituent that is a monovalent alkyl having one hydrogen atom replaced with a valency. An optionally substituted alkylene is an alkylene that is optionally substituted as described herein for alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, represents a divalent substituent that is an aryl having one hydrogen atom replaced with a valency. An optionally substituted arylene is an arylene that is optionally substituted as described herein for aryl.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “bicyclic sugar moiety,” as used herein, represents a modified sugar moiety including two fused rings. In certain embodiments, the bicyclic sugar moiety includes a furanosyl ring.

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₆₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine) and Hoogsteen base pairs.

The term “contiguous,” as used herein in the context of an oligonucleotide, refers to nucleosides, nucleobases, sugar moieties, or internucleoside linkages that are immediately adjacent to each other. For example, “contiguous nucleobases” means nucleobases that are immediately adjacent to each other in a sequence.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, represents a divalent substituent that is a cycloalkyl having one hydrogen atom replaced with a valency. An optionally substituted cycloalkylene is a cycloalkylene that is optionally substituted as described herein for cycloalkyl.

The term “cycloalkoxy,” as used herein, represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “duplex,” as used herein, represents two oligonucleotides that are paired through hybridization of complementary nucleobases.

The term “exon 8,” as used herein, refers to exon 8 of LIPA pre-mRNA or genomic DNA, e.g., SEQ ID NO: 2, which corresponds to positions 34322 to 34393 in SEQ ID NO: 1 (hg19/b37 coordinates chr13: 90982268-90982339), or a mutant version thereof (e.g., g.34393G>A in SEQ ID NO: 1).

The term “flanking intron,” as used herein, refers to an intron that is adjacent to the 5′- or 3′-end of a LIPA exon (e.g., exon 8) or a mutant thereof. The flanking intron is a 5′-flanking intron or a 3′-flanking intron. The 5′-flanking intron corresponds to the flanking intron that is adjacent to the 5′-end of the exon (e.g., exon 8) targeted for inclusion. In some embodiments, the 5′-flanking intron is disposed between exon 7 and exon 8 in SEQ ID NO: 1. The 3′-flanking intron corresponds to the flanking intron that is adjacent to the 3′-end of the exon (e.g., exon 8) targeted for inclusion. In some embodiments, the 3′-flanking intron is disposed between exon 8 and exon 9 in SEQ ID NO: 1).

The term “genetic aberration,” as used herein, generally refers to a mutation or variant in a gene. Examples of genetic aberration may include, but are not limited to, a point mutation (single nucleotide variant or single base substitution), an insertion or deletion (indel), a transversion, a translocation, an inversion, or a truncation. An aberrant LIPA gene may include one or more mutations causing the splicing of pre-mRNA to skip an exon in the LIPA gene (e.g., exon 8).

The term “halo,” as used herein, represents a halogen selected from bromine, chlorine, iodine, and fluorine.

The term “heteroalkane-tetrayl,” as used herein refers to an alkane-tetrayl group interrupted once by one heteroatom; twice, each time, independently, by one heteroatom; three times, each time, independently, by one heteroatom; or four times, each time, independently, by one heteroatom. Each heteroatom is, independently, O, N, or S. In some embodiments, the heteroatom is O or N. An unsubstituted C_{x-y} heteroalkane-tetrayl contains from X to Y carbon atoms as well as the heteroatoms as defined herein. The heteroalkane-tetrayl group may be unsubstituted or substituted (e.g., optionally substituted heteroalkane-tetrayl), as described for heteroalkyl.

The term “heteroalkane-triyl,” as used herein refers to an alkane-triyl group interrupted once by one heteroatom; twice, each time, independently, by one heteroatom; three times, each time, independently, by one heteroatom; or four times, each time, independently, by one heteroatom. Each heteroatom is, independently, O, N, or S. In some embodiments, the heteroatom is O or N. An unsubstituted C_{x-y} heteroalkane-triyl contains from X to Y carbon atoms as well as the heteroatoms as defined herein. The heteroalkane-triyl group may be unsubstituted or substituted (e.g., optionally substituted heteroalkane-triyl), as described for heteroalkyl.

The term “heteroalkyl,” as used herein, refers to an alkyl group interrupted one or more times by one or two heteroatoms each time. Each heteroatom is independently O, N, or S. None of the heteroalkyl groups includes two contiguous oxygen atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. In some embodiments, carbon atoms are found at the termini of a heteroalkyl group. In some embodiments, heteroalkyl is PEG.

The term “heteroalkylene,” as used herein, represents a divalent substituent that is a heteroalkyl having one hydrogen atom replaced with a valency. An optionally substituted heteroalkylene is a heteroalkylene that is optionally substituted as described herein for heteroalkyl.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroarylene,” as used herein, represents a divalent substituent that is a heteroaryl having one hydrogen atom replaced with a valency. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkoxy; acyloxy; aryloxy; amino; arylalkoxy; cycloalkyl; cycloalkoxy; halogen; heterocyclyl;

heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclylene,” as used herein, represents a divalent substituent that is a heterocyclyl having one hydrogen atom replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The term “heteroorganic,” as used herein, refers to (i) an acyclic hydrocarbon interrupted one or more times by one or two heteroatoms each time, or (ii) a cyclic hydrocarbon including one or more (e.g., one, two, three, or four) endocyclic heteroatoms. Each heteroatom is independently O, N, or S. None of the heteroorganic groups includes two contiguous oxygen atoms. An optionally substituted heteroorganic group is a heteroorganic group that is optionally substituted as described herein for alkyl.

The term “hydrocarbon,” as used herein, refers to an acyclic, branched or acyclic, linear compound or group, or a monocyclic, bicyclic, tricyclic, or tetracyclic compound or group. The hydrocarbon, when unsubstituted, consists of carbon and hydrogen atoms. Unless specified otherwise, an unsubstituted hydrocarbon includes a total of 1 to 60 carbon atoms (e.g., 1 to 16, 1 to 12, or 1 to 6 carbon atoms). An optionally substituted hydrocarbon is an optionally substituted acyclic hydrocarbon or an optionally substituted cyclic hydrocarbon. An optionally substituted acyclic hydrocarbon is optionally substituted as described herein for alkyl. An optionally substituted cyclic hydrocarbon is an optionally substituted aromatic hydrocarbon or an optionally substituted non-aromatic hydrocarbon. An optionally substituted aromatic hydrocarbon is optionally substituted as described herein for aryl. An optionally substituted non-aromatic cyclic hydrocarbon is optionally substituted as described herein for cycloalkyl. In some embodiments, an acyclic hydrocarbon is alkyl, alkylene, alkane-triyl, or alkane-tetrayl. In certain embodiments, a cyclic hydrocarbon is aryl or arylene. In particular embodiments, a cyclic hydrocarbon is cycloalkyl or cycloalkylene.

The terms “hydroxyl” and “hydroxy,” as used interchangeably herein, represent —OH.

The term “hydrophobic moiety,” as used herein, represents a monovalent group covalently linked to an oligonucleotide backbone, where the monovalent group is a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen. Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ hydrocarbon (e.g., optionally substituted C₁₋₆₀ alkylene) or an optionally substituted C₂₋₆₀ heteroorganic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

The term “internucleoside linkage,” as used herein, represents a divalent group or covalent bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester. The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, phosphorodithioate linkages, boranophosphonate linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

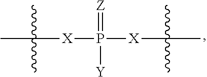

where

Z is O, S, B, or Se;

Y is —X—L—R¹;

each X is independently —O—, —S—, —N(—L—R¹)—, or L;

each L is independently a covalent bond or a linker (e.g., optionally substituted C₁₋₆₀ hydrocarbon linker or optionally substituted C₂₋₆₀ heteroorganic linker);

each R¹ is independently hydrogen, —S—S—R², —O—CO—R², —S—CO—R², optionally substituted C₁₋₉ heterocyclyl, a hydrophobic moiety, or a targeting moiety; and

each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl.

When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either (1) L is a linker or (2) R¹ is not a hydrogen, the internucleoside group is known as a phosphotriester. When Z is sulfur, all X groups are —O—, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

The term “LIPA” or “LAL,” as used herein, represents a nucleic acid (e.g., genomic DNA, pre-mRNA, or mRNA) that is translated and, if genomic DNA, first transcribed, in vivo to Lysosomal Acid Lipase protein. An exemplary genomic DNA sequence comprising the human LIPA gene is given by SEQ ID NO: 1 (NCBI Reference Sequence: NG_008194.1). SEQ ID NO: 1 provides the sequence for the antisense strand of the genomic DNA of LIPA (positions 4865-43335 in SEQ ID NO: 1). One of skill in the art will recognize that an RNA sequence typically includes uridines instead of thymidines. The term “LIPA” or “LAL,” as used herein, represents wild-type and mutant versions. An exemplary mutant nucleic acid (e.g., genomic DNA, pre-mRNA, or mRNA) results in Lysosomal Acid Lipase protein lacking exon 8.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

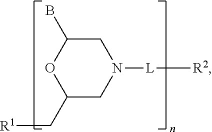

where

n is an integer of at least 10 (e.g., 12 to 50) indicating the number of morpholino units;

each B is independently a nucleobase;

R¹ is a 5′ group;

R² is a 3′ group; and

L is (i) a morpholino internucleoside linkage or, (ii) if L is attached to R², a covalent bond. A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer. A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer.

The term “morpholino internucleoside linkage,” as used herein, represents a divalent group of the following structure.

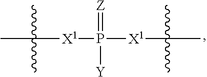

where

Z is O or S;

X¹ is a bond, —CH₂—, or —O—;

X² is a bond, —CH₂—O—, or —O—; and

Y is —NR₂, where each R is independently C₁₋₆ alkyl (e.g., methyl), or both R combine together with the nitrogen atom to which they are attached to form a C₂₋₉ heterocyclyl (e.g., N-piperazinyl);

provided that both X¹ and X² are not simultaneously a bond.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or “natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or O6-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in U.S. Pat. No. 3,687,808; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art (e.g., modified or unmodified ribofuranose-nucleobase and 2′-deoxyribofuranose-nucleobase compounds and groups known in the art). The sugar may be ribofuranose. The sugar may be modified or unmodified. An unmodified sugar nucleoside is ribofuranose or 2′-deoxyribofuranose having an anomeric carbon bonded to a nucleobase. An unmodified nucleoside is ribofuranose or 2′-deoxyribofuranose having an anomeric carbon bonded to an unmodified nucleobase. Non-limiting examples of unmodified nucleosides include adenosine, cytidine, guanosine, uridine, 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

The term “nucleotide,” as used herein, represents a nucleoside bonded to an internucleoside linkage or a monovalent group of the following structure —X¹—P(X²)(R¹)^{2,} where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more (e.g., 10 to 50) contiguous nucleosides covalently bound together by internucleoside linkages. An oligonucleotide includes a 5′ end and a 3′ end. The 5′ end of an oligonucleotide may be, e.g., hydroxyl, a targeting moiety, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a targeting moiety, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus.

The term “oxo,” as used herein, represents a divalent oxygen atom (e.g., the structure of oxo may be shown as ═O).

The term “pharmaceutically acceptable,” as used herein, refers to those compounds, materials, compositions, and/or dosage forms, which are suitable for contact with the tissues of an individual (e.g., a human), without excessive toxicity, irritation, allergic response and other problem complications commensurate with a reasonable benefit/risk ratio.

The term “protecting group,” as used herein, represents a group intended to protect a functional group (e.g., a hydroxyl, an amino, or a carbonyl) from participating in one or more undesirable reactions during chemical synthesis. The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Wuts, “Greene's Protective Groups in Organic Synthesis,” 4th Edition (John Wiley & Sons, New York, 2006), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and arylalkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl;

dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydroxy carbonyl, t-butyloxycarbonyl, diisopropylmethoxycarbonyl, isopropoxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like.

The term “pyrid-2-yl hydrazone,” as used herein, represents a group of the structure:

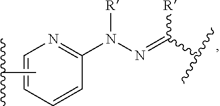

where each R′ is independently H or optionally substituted C₁₋₆ alkyl. Pyrid-2-yl hydrazone may be unsubstituted (i.e., each R′ is H).

The term “splice site,” as used herein, generally refers to a site in a genome corresponding to an end of an intron that may be involved in a splicing procedure. A splice site may be a 5′ splice site (e.g., a 5′ end of an intron) or a 3′ splice site (e.g., a 3′ end of an intron). A given 5′ splice site may be associated with one or more candidate 3′ splice sites, each of which may be coupled to its corresponding 5′ splice site in a splicing operation.

The term “splicing enhancer,” as used herein, refers to motifs with positive effects (e.g., causing an increase) on exon inclusion.

The term “splicing regulatory element,” as used herein, refers to an exonic splicing silencer element, an exonic splicing enhancer element, an intronic splicing silencer element, and an an intronic splicing enhancer element. An exonic splicing silencer element is a portion of the target pre-mRNA exon that reduces the ratio of transcripts including this exon relative to the total number of the gene transcripts. An intronic splicing silencer element is a portion of the target pre-mRNA intron that reduces the ratio of transcripts including the exon adjacent to the target intron relative to the total number of the gene transcripts. An exonic splicing enhancer element is a portion of the target pre-mRNA exon that increases the ratio of transcripts including this exon relative to the total number of the gene transcripts. An intronic splicing enhancer element is a portion of the target pre-mRNA intron that increases the ratio of transcripts including the exon adjacent to the target intron relative to the total number of the gene transcripts.

The term “splicing silencer,” as used herein, refers to motifs with negative effects (e.g., causing a decrease) on exon inclusion.

The term “stereochemically enriched,” as used herein, refers to a local stereochemical preference for one enantiomer of the recited group over the opposite enantiomer of the same group. Thus, an oligonucleotide containing a stereochemically enriched internucleoside linkage is an oligonucleotide in which a stereogenic internucleoside linkage (e.g., phosphorothioate) of predetermined stereochemistry is present in preference to a stereogenic internucleoside linkage (e.g., phosphorothioate) of stereochemistry that is opposite of the predetermined stereochemistry. This preference can be expressed numerically using a diastereomeric ratio for the stereogenic internucleoside linkage (e.g., phosphorothioate) of the predetermined stereochemistry. The diastereomeric ratio for the stereogenic internucleoside linkage (e.g., phosphorothioate) of the predetermined stereochemistry is the molar ratio of the diastereomers having the identified stereogenic internucleoside linkage (e.g., phosphorothioate) with the predetermined stereochemistry relative to the diastereomers having the identified stereogenic internucleoside linkage (e.g., phosphorothioate) with the stereochemistry that is opposite of the predetermined stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “subject,” as used herein, represents a human or non-human animal (e.g., a mammal) that is suffering from, or is at risk of, disease, disorder, or condition, as determined by a qualified professional (e.g., a doctor or a nurse practitioner) with or without known in the art laboratory test(s) of sample(s) from the subject. A non-limiting example of a disease, disorder, or condition includes Wolman Disease or Cholesteryl Ester Storage Disease (e.g., Wolman Disease or Cholesteryl Ester Storage Disease associated with exon 8 skipping).

A “sugar” or “sugar moiety,” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-5-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

The term “targeting moiety,” as used herein, represents a moiety (e.g., N-acetylgalactosamine or a cluster thereof) that specifically binds or reactively associates or complexes with a receptor or other receptive moiety associated with a given target cell population. An antisense oligonucleotide may contain a targeting moiety. An antisense oligonucleotide including a targeting moiety is also referred to herein as a conjugate. A targeting moiety may include one or more ligands (e.g., 1 to 6 ligands, 1 to 3 ligands, or 1 ligand). The ligand can be an antibody or an antigen-binding fragment or an engineered derivative thereof (e.g., Fcab or a fusion protein (e.g., scFv)). Alternatively, the ligand may be a small molecule (e.g., N-acetylgalactosamine).

The term “therapeutically effective amount,” as used herein, represents the quantity of an antisense oligonucleotide of the invention necessary to ameliorate, treat, or at least partially arrest the symptoms of a disease or disorder (e.g., to increase the level of LIPA mRNA molecules including the otherwise skipped exon (e.g., exon 8)). Amounts effective for this use may depend, e.g., on the severity of the disease and the weight and general state of the subject. Typically, dosages used in vitro may provide useful guidance in the amounts useful for in vivo administration of the pharmaceutical composition, and animal models may be used to determine effective dosages for treatment of particular disorders. In some embodiments, a therapeutically effective amount of an antisense oligonucleotide of the invention reduces the plasma triglycerides level, e.g., at least 5%, at least 10%, at least 15%, at least 20%, at least 25%, at least 30%, at least 35%, at least 40%, at least 45%, or at least 50%; e.g., up to 80%, up to 70%, up to 60%, up to 50%, up to 40%, up to 30%, or up to 20%, as compared to the plasma triglycerides level prior to the administration of an antisense oligonucleotide. In some embodiments, a therapeutically effective amount of an antisense oligonucleotide of the invention reduces or maintains the plasma triglyceride levels in the subject to 300 mg/dL or less, 250 mg/dL or less, 200 mg/dL or less, or to 150 mg/dL or less. In some embodiments, a therapeutically effective amount of an antisense oligonucleotide of the invention reduces the plasma low density lipoprotein (LDL-C) level, e.g., at least 5%, at least 10%, at least 15%, at least 20%, at least 25%, at least 30%, at least 35%, at least 40%, at least 45%, or at least 50%; e.g., up to 80%, up to 70%, up to 60%, up to 50%, up to 40%, up to 30%, or up to 20%, as compared to the LDL-C level prior to the administration of an antisense oligonucleotide. In some embodiments, a therapeutically effective amount of an antisense oligonucleotide of the invention reduces or maintains the plasma LDL-C levels in the subject to less than 300 mg/dL, less than 250 mg/dL, less than 200 mg/dL, less than 190 mg/dL, less than 160 mg/dL, less than 150 mg/dL, less than 130 mg/dL, or less than 100 mg/dL. Lipid levels can be assessed using plasma lipid analyses or tissue lipid analysis. In plasma lipid analysis, blood plasma can be collected, and total plasma free cholesterol levels can be measured using, for example colorimetric assays with a COD-PAP kit (Wako Chemicals), total plasma triglycerides can be measured using, for example, a Triglycerides/GB kit (Boehringer Mannheim), and/or total plasma cholesterol can be determined using a Cholesterol/HP kit (Boehringer Mannheim). In tissue lipid analysis, lipids can be extracted, for example, from liver, spleen, and/or small intestine samples (e.g., using the method in Folch et al. J. Biol. Chem 226: 497-505 (1957)). Total tissue cholesterol concentrations can be measured, for example, using O-phthalaldehyde.

The term “thiocarbonyl,” as used herein, represents a C(═S) group. Non-limiting example of functional groups containing a “thiocarbonyl” includes thioesters, thioketones, thioaldehydes, thioanhydrides, thioacyl chlorides, thioamides, thiocarboxylic acids, and thiocarboxylates.

The term “thioheterocyclylene,” as used herein, represents a divalent group —S—R′—, where R′ is a heterocyclylene as defined herein.

The term “thiol,” as used herein, represents an —SH group.

The term “triazolocycloalkenylene,” as used herein, refers to the heterocyclylenes containing a 1,2,3-triazole ring fused to an 8-membered ring, all of the endocyclic atoms of which are carbon atoms, and bridgehead atoms are sp²-hybridized carbon atoms. Triazocycloalkenylenes can be optionally substituted in a manner described for heterocyclyl.

The term “triazoloheterocyclylene,” as used herein, refers to the heterocyclylenes containing a 1,2,3-triazole ring fused to an 8-membered ring containing at least one heteroatom. The bridgehead atoms in triazoloheterocyclylene are carbon atoms. Triazoloheterocyclylenes can be optionally substituted in a manner described for heterocyclyl.

Enumeration of positions within oligonucleotides and nucleic acids, as used herein and unless specified otherwise, starts with the 5′-terminal nucleoside as 1 and proceeds in the 3′-direction.

The compounds described herein, unless otherwise noted, encompass isotopically enriched compounds (e.g., deuterated compounds), tautomers, and all stereoisomers and conformers (e.g. enantiomers, diastereomers, EIZ isomers, atropisomers, etc.), as well as racemates thereof and mixtures of different proportions of enantiomers or diastereomers, or mixtures of any of the foregoing forms as well as salts (e.g., pharmaceutically acceptable salts).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

FIGS. 1A and 1B show that the chr10:90982268:C:T [hg19/b37] variant reduces exon 8 inclusion in GM03111 fibroblasts. FIG. 1A is a schematic of the effect of the target variant on splicing of LIPA exon 8. FIG. 1B shows RT-PCR analysis in apparently healthy (GM00288 and GM09503) and LIPA-variant (GM03111 and GM00863) fibroblasts. Exon inclusion (165 bp) and exclusion (92 bp) products are indicated by arrows. 100 bp DNA ladder is shown for size reference.

FIG. 2 shows that LIPA enzyme activity is decreased in fibroblasts containing the LIPA variant c.894G>A (GM03111), measured by its ability to cleave of the fluorogenic substrate, 4-methylumberlliferyl oleate. Mean enzyme activity is presented in arbitrary units, error bars represent standard deviation in 2 technical replicates.

FIG. 3 shows representative capillary electrophoresis of RT-PCR products of GM03111 fibroblasts transfected with antisense oligonucleotides having the sequences set forth in SEQ ID Nos: 7, 3-14, 31-58, and 75-91. In FIG. 3, DG-FAM (Eurofins Genomics, Louisville, Ky.) refers to a conjugate of an M13 primer sequence and an FAM moiety used as an antisense oligonucleotide. The exon 8 inclusion band is at 165 bp and the exclusion band at 92 bp.

FIG. 4 shows viability of HepG2 cells transfected with antisense oligonucleotide compared to PSI in LIPA c.894G>A fibroblasts. In FIG. 4, each dot represents an antisense oligonucleotide identified by its SEQ ID NO.

FIG. 5 shows the rescue of LIPA enzyme activity in LIPA mutant fibroblasts by antisense oligonucleotide SEQ ID NO. 84. Error bars represent standard deviation in 4 replicates.

### DETAILED DESCRIPTION

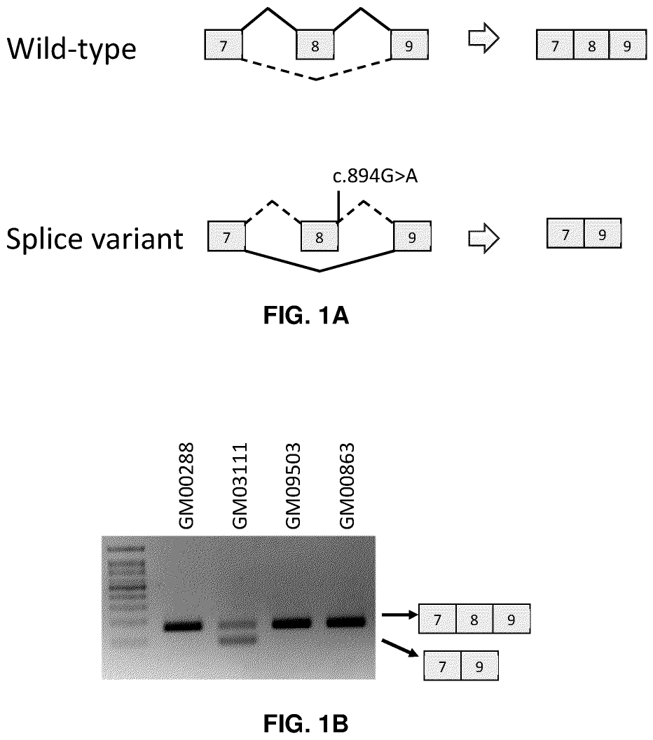

In general, the present invention provides antisense oligonucleotides, compositions, and methods that target a LIPA exon (e.g., exon 8) or a flanking intron. Surprisingly, the inventors have found that altering LIPA gene splicing to promote inclusion of an otherwise skipped exon (e.g., exon 8) in the transcript of splice variants (FIG. 1A) may be used to treat Wolman Disease or Cholesteryl Ester Storage Disease, and antisense oligonucleotides may be used to alter splicing of the LIPA gene to include the otherwise skipped exon (e.g., exon 8). The antisense oligonucleotides of the invention may modulate splicing of LIPA pre-mRNA to increase the level of LIPA mRNA molecules having the otherwise skipped exon (e.g., exon 8). Accordingly, the antisense oligonucleotides may be used to treat Wolman Disease or Cholesteryl Ester Storage Disease in a subject in need of a treatment therefor. Typically, an antisense oligonucleotide includes a nucleobase sequence at least 70% (e.g., at least 80%, at least 90%, at least 95%, or 100%) complementary to a LIPA pre-mRNA sequence in a 5′-flanking intron, a 3′-flanking intron, or a combination of an exon (e.g., exon 8) and a 5′-flanking or 3′-flanking intron (e.g., a 5′-flanking or 3′-flanking intron adjacent to exon 8).

Genetic variants may correspond to changes or modifications in transcription and/or splicing. RNA is initially transcribed from DNA as pre-mRNA, with protein-coding and 5′UTR/3′UTR exons separated by introns. Splicing generally refers to the molecular process, carried out by the spliceosome complexes that may remove introns and adjoins exons, producing a mature mRNA sequence, which is then scanned and translated to protein by the ribosome. The molecular reaction catalyzed by the spliceosome may comprise (i) nucleophilic attack of the branch site adenosine 2′0H onto the outmost base of the intronic donor dinucleotide, with consequent release of the outmost exonic donor base 3′OH; and (ii) nucleophilic attack of the exonic donor 3′OH onto the outmost exonic acceptor base, with consequent release of the intron lariat and the spliced exons.

Advantageously, the antisense oligonucleotides described herein may exhibit reduced or no toxicity. A combination of the targeting moiety, nucleobase sequence, nucleoside modifications, and/or internucleoside linkage modification may provide the reduced or no toxicity effect and/or enhance pharmacokinetics without sacrificing antisense activity. Without wishing to be bound by theory, favorable toxicity and pharmacokinetic properties of the antisense oligonucleotides described herein may be due to their efficient targeting to hepatocytes and/or Kupffer cells. The hepatocyte and/or Kupffer cell targeting efficiency may be measured using techniques and methods known in the art, e.g., an RNA in situ hybridization.

Splicing sequence changes can include the following categories: (a) alteration of a splice site (denominated canonical splice site) or exon recognition sequence required for the proper composition of a gene product, and (b) activation and utilization of an incorrect splice site (denominated cryptic splice site), or incorrect recognition of intronic sequence as an exon (denominated pseudo exon). Both (a) and (b) may result in the improper composition of a gene product. The splice site recognition signal may be required for spliceosome assembly and can comprise the following structures: (i) highly conserved intronic dinucleotide (AG, GT) immediately adjacent to the exon-intron boundary, and (ii) consensus sequence surrounding the intronic dinucleotide (often delimited to 3 exonic and 6 intronic nucleotides for the donor site, 3 exonic and 20 intronic nucleotides for the acceptor site) and branch site (variable position on the intronic acceptor side), both with lower conservation and more sequence variety.

In addition to splice site recognition, the exon recognition signal may comprise a plethora of motifs recognized by splicing factors and other RNA binding proteins, some of which may be ubiquitously expressed and some of which may be tissue specific. These motifs may be distributed over the exon body and in the proximal intronic sequence. The term “splicing enhancer” refers to motifs with positive effects (e.g., causing an increase) on exon inclusion, and the term “splicing silencer” refers to motifs with negative effects (e.g., causing a decrease) on exon inclusion. The exon recognition signal may be particularly important for correct splicing in the presence of weak consensus sequence. When a variant weakens the splice site recognition, the exon can be skipped and/or a nearby cryptic splice site which is already fairly strong can be used. In the presence of short introns, full intron retention is also a possible outcome. In particular, alteration of the intronic dinucleotide often results in splicing alteration, whereas consensus sequence alteration may be, on average, less impactful and more context-dependent. When the exon recognition signal is weakened, exon skipping may be a more likely outcome, but cryptic splice site use is also possible, especially in the presence of a very weak consensus sequence. Variants can also strengthen a weak cryptic splice site in proximity of the canonical splice site, and significantly increase its usage resulting in improper splicing and incorrect gene product (with effects including amino acid insertion/deletion, frameshift, and stop-gain).

Antisense oligonucleotides can be used to modulate gene splicing (e.g., by targeting splicing regulatory elements of the gene).

Antisense oligonucleotides may comprise splice-switching oligonucleotides (SSOs), which may modulate splicing by steric blockage preventing the spliceosome assembly or the binding of splicing factors and RNA binding proteins. Blocking binding of specific splicing factors or RNA binding proteins that have an inhibitory effect may be used to produce increased exon inclusion (e.g., exon 8 inclusion). Specific steric blocker antisense oligonucleotide chemistries may include the modified RNA chemistry with phosphorothioate backbone (PS) with a sugar modification (e.g., 2′-modification) and phosphorodiamidate morpholino (PMO). Exemplary PS backbone sugar modifications may include 2′-O-methyl (2′OMe) and 2′-O-methoxyethyl (2′-MOE), which is also known as 2′-methoxyethoxy. Other nucleotide modifications may be used, for example, for the full length of the oligonucleotide or for specific bases. The oligonucleotides can be covalently conjugated to a targeting moiety (e.g., a GaINAc cluster), or to a peptide (e.g., a cell penetrating peptide), or to another molecular or multimolecular group (e.g., a hydrophobic moiety or neutral polymer) different from the rest of the oligonucleotide. Antisense oligonucleotides may be used as a single stereoisomer or a combination of stereoisomers.

The LIPA gene (lipase A, lysosomal acid type, Entrez Gene ID 3988) may play an important role in the pathogenicity of Wolman Disease and Cholesteryl Ester Storage Disease. LIPA is a gene encoding a lysosomal enzyme required for the hydrolysis of cholesteryl esters and triglycerides, which are derived from the internalization of plasma lipoprotein particles (chylomicron remnants, LDL, IDL) by endocytosis. The gene may be expressed in liver hepatocytes. Defective hydrolysis of cholesteryl esters and triglycerides can lead to toxic effects in the liver. LIPA homozygous or compound heterozygous loss-of-function may result in the autosomal recessive Wolman Disease, partial loss-of-function may result in Cholesteryl Ester Storage Disease.

Recognizing a need for effective splicing modulation therapies for diseases such as Wolman Disease or Cholesteryl Ester Storage Disease, the present disclosure provides LIPA splice-modulating antisense oligonucleotides comprising sequences targeted to an intron adjacent to an abnormally spliced exon (e.g., exon 8) of LIPA. In some embodiments, the antisense oligonucleotide has a sequence targeted to one or more splicing regulatory elements which may be located in an intron adjacent to an abnormally spliced exon (e.g., exon 8) of LIPA. The present disclosure also provides methods for modulating splicing of LIPA RNA in a cell, tissue, or organ of a subject by bringing the cell, tissue, or organ in contact with an antisense oligonucleotide of the invention. A LIPA splice-modulating antisense oligonucleotide may comprise a nucleobase sequence targeted to a splicing regulatory element of an intron adjacent to an abnormally spliced exon (e.g., exon 8) of LIPA. In addition, the present disclosure provides a method for treating Wolman Disease or Cholesteryl Ester Storage Disease in a subject by administering to the subject a therapeutically effective amount of an oligonucleotide of the invention. A LIPA splice-modulating antisense oligonucleotide may comprise a sequence targeted to a splicing regulatory element of or an intron adjacent to an abnormally spliced exon (e.g., exon 8) of LIPA.

Splicing regulatory elements may include, for example, exonic splicing silencer elements or intronic splicing silencer elements. The antisense oligonucleotides may comprise sequences targeted to an intron adjacent to the exon (e.g., exon 8) of LIPA which modulates variant splicing of LIPA RNA. The modulation of splicing may result in an increase in exon inclusion (e.g., exon 8 inclusion). Antisense oligonucleotides may comprise a total of 8 to 50 nucleotides (e.g., 8 to 16 nucleotides, 8 to 20 nucleotides, 12 to 20 nucleotides, 12 to 30 nucleotides, or 12 to 50 nucleotides).

Genetic aberrations of the LIPA gene may play an important role in pathogenicity. In particular, a LIPA chr10:90982268:C:T [hg19/b37] genetic aberration (g.34393G>A mutant of SEQ ID NO: 1), may result in NM_000235.3(LIPA) cDNA change c.894G>A and no change in the protein sequence at amino acid position 298 (GIn) in exon 8. Genome coordinates may be expressed, for example, with respect to human genome reference hg19/b37. For example, this variant has been reported as pathogenic in patients with Wolman Disease or Cholesteryl Ester Storage Disease.

These exemplary genetic aberrations may be targeted with antisense oligonucleotides to increase levels of exon inclusion (e.g., exon 8 inclusion).

Different antisense oligonucleotides can be combined for increasing an exon inclusion (e.g., exon 8 inclusion) of LIPA. A combination of two antisense oligonucleotides may be used in a method of the invention, such as two antisense oligonucleotides, three antisense oligonucleotides, four different antisense oligonucleotides, or five different antisense oligonucleotides targeting the same or different regions or “hotspots.”

An antisense oligonucleotide according to the invention may be indirectly administered using suitable techniques and methods known in the art. It may for example be provided to an individual or a cell, tissue or organ of the individual in the form of an expression vector wherein the expression vector encodes a transcript comprising said oligonucleotide. The expression vector is preferably introduced into a cell, tissue, organ or individual via a gene delivery vehicle. In an embodiment, there is provided a viral based expression vector comprising an expression cassette or a transcription cassette that drives expression or transcription of an antisense oligonucleotide as identified herein. Accordingly, the present invention provides a viral vector expressing an antisense oligonucleotide according to the invention.

An antisense oligonucleotide according to the invention may be directly administered using suitable techniques and methods known in the art, e.g., using conjugates described herein.

### Conjugates

Oligonucleotides of the invention may include an auxiliary moiety, e.g., a targeting moiety, hydrophobic moiety, cell penetrating peptide, or a polymer. An auxiliary moiety may be present as a 5′ terminal modification (e.g., covalently bonded to a 5′-terminal nucleoside), a 3′ terminal modification (e.g., covalently bonded to a 3′-terminal nucleoside), or an internucleoside linkage (e.g., covalently bonded to phosphate or phosphorothioate in an internucleoside linkage).

### Targeting Moieties

An oligonucleotide of the invention may include a targeting moiety.

A targeting moiety is selected based on its ability to target oligonucleotides of the invention to a desired or selected cell population that expresses the corresponding binding partner (e.g., either the corresponding receptor or ligand) for the selected targeting moiety. For example, an oligonucleotide of the invention could be targeted to hepatocytes expressing asialoglycoprotein receptor (ASGP-R) by selecting a targeting moiety containing N-acetylgalactosamine (GaINAc).

A targeting moiety may include one or more ligands (e.g., 1 to 9 ligands, 1 to 6 ligands, 1 to 3 ligands, 3 ligands, or 1 ligand). The ligand may target a cell expressing asialoglycoprotein receptor (ASGP-R), IgA receptor, HDL receptor, LDL receptor, or transferrin receptor. Non-limiting examples of the ligands include N-acetylgalactosamine, glycyrrhetinic acid, glycyrrhizin, lactobionic acid, lactoferrin, IgA, or a bile acid (e.g., lithocholyltaurine or taurocholic acid).

The ligand may be a small molecule, e.g., a small molecules targeting a cell expressing asialoglycoprotein receptor (ASGP-R). A non-limiting example of a small molecule targeting an asialoglycoprotein receptor is N-acetylgalactosamine. Alternatively, the ligand can be an antibody or an antigen-binding fragment or an engineered derivative thereof (e.g., Fcab or a fusion protein (e.g., scFv)).

A targeting moiety may be -LinkA(-T)ₚ, where LinkA is a multivalent linker, each T is a ligand (e.g., asialoglycoprotein receptor-targeting ligand (e.g., N-acetylgalactosamine)), and p is an integer from 1 to 9. When each T is N-acetylgalactosamine, the targeting moiety is referred to as a galactosamine cluster. Galactosamine clusters that may be used in oligonucleotides of the invention are known in the art. Non-limiting examples of the galactosamine clusters that may be included in the oligonucleotides of the invention are provided in U.S. Pat. Nos. 5,994,517; 7,491,805; 9,714,421; 9,867,882; 9,127,276; US 2018/0326070; US 2016/0257961; WO 2017/100461; and in Sliedregt et al., J. Med. Chem., 42:609-618, 1999. Ligands other than GaINAc may also be used in clusters, as described herein for galactosamine clusters.

Targeting moiety -LinkA(-T)p may be a group of formula (I):

$$\text{-} {Q}^{1} \text{-} {{Q}^{2} ( {[ \text{-} {Q}^{3} \text{-} {Q}^{4} \text{-} {Q}^{5} ]}_{s} \text{-} {Q}^{6} \text{-} T )}_{p} , \qquad ( I )$$

where

each s is independently an integer from 0 to 20 (e.g., from 0 to 10), where the repeating units are the same or different;

Q¹ is a conjugation linker (e.g., [-Q³-Q⁴-Q⁵]ₛ-Q^{c}-, where Q^{C} is optionally substituted C₂₋₁₂ heteroalkylene (e.g., a heteroalkylene containing —C(O)—N(H)—, —N(H)—C(O)—, —S(O)₂—N(H)—, —N(H)—S(O)₂—, or —S—S—), optionally substituted C₁₋₁₂ thioheterocyclylene

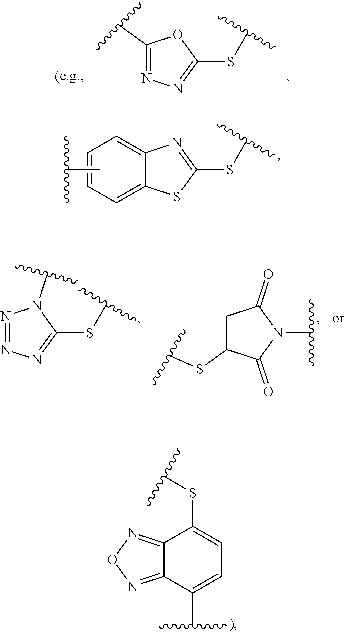

optionally substituted C₁₋₁₂ heterocyclylene (e.g., 1,2,3-triazole-1,4-diyl or

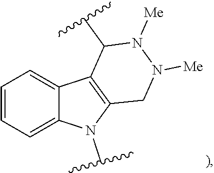 [SMILES: *C1c2c(n(*)c3ccccc23)CN(C)N1C]

cyclobut-3-ene-1,2-dione-3,4-diyl, pyrid-2-yl hydrazone, optionally substituted C₆₋₁₆ triazoloheterocyclylene

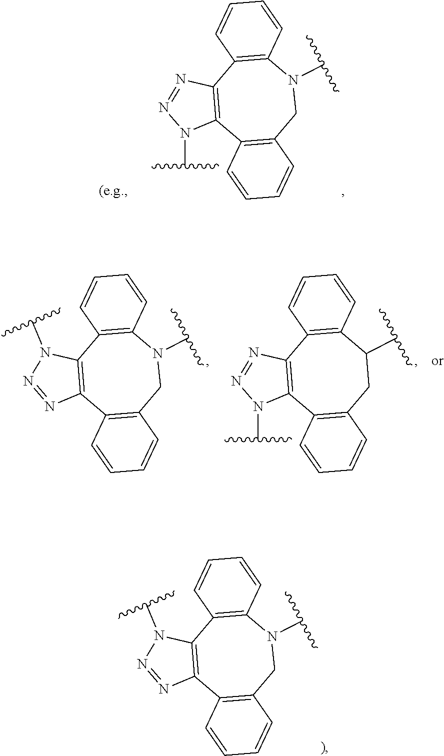 [SMILES: *N1Cc2ccccc2-c2c(nnn2*)-c2ccccc21 ; *N1Cc2ccccc2-c2nnn(*)c2-c2ccccc21 ; *C1Cc2ccccc2-c2c(nnn2*)-c2ccccc21 ; *N1Cc2ccccc2-c2nnn(*)c2-c2ccccc21]

optionally substituted C₈₋₁₆ triazolocycloalkenylene

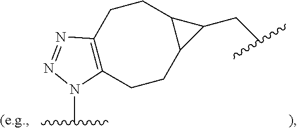 [SMILES: *CC1C2CCc3nnn(*)c3CCC12]

or a dihydropyridazine group

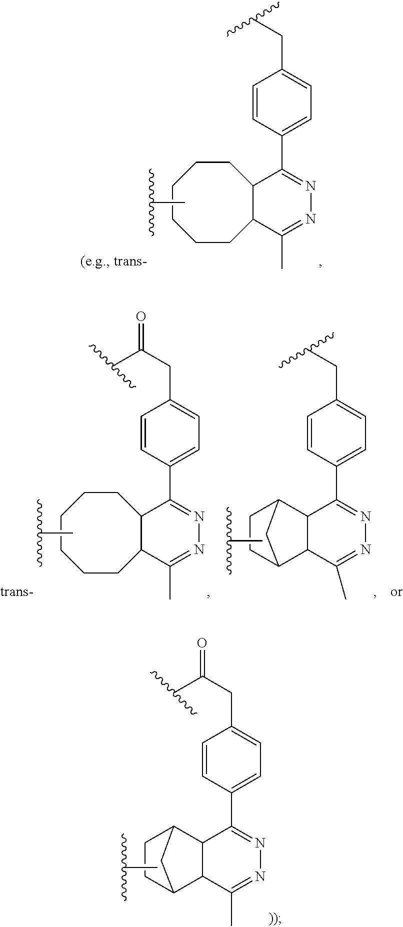

Q² is a linear group (e.g., [-Q³-Q⁴-Q⁵]ₛ-), if p is 1, or a branched group (e.g., [-Q³-Q⁴-Q⁵]ₛ-Q⁷([-Q³-Q⁴-Q⁵]ₛ-(Q⁷)ₚ₁)ₚ₂, where p1 is 0, 1, or 2, and p2 is 0, 1, 2, or 3), if p is an integer from 2 to 9;

each Q³ and each Q⁶ is independently absent, —CO—, —NH—, —O—, —S—, —SO₂—, —OC(O)—, —C(O)O—, —NHC(O)—, —C(O)NH—, —CH₂—, —CH₂NH—, —NHCH₂—, —CH₂O—, or —OCH₂—;

each Q⁴ is independently absent, optionally substituted C₁₋₁₂ alkylene, optionally substituted C₂₋₁₂ alkenylene, optionally substituted C₂₋₁₂ alkynylene, optionally substituted C₂₋₁₂ heteroalkylene, optionally substituted C₆₋₁₀ arylene, optionally substituted C₁₋₉ heteroarylene, or optionally substituted C₁₋₉ heterocyclylene;

each Q⁵ is independently absent, —CO—, —NH—, —O—, —S—, —SO₂—, —CH₂—, —C(O)O—, —OC(O)—, —C(O)NH—, —NH—C(O)—, —NH—CH(R^{a})—C(O)—, —C(O)—CH(R^{a})—NH—, —OP(O)(OH)O—, or —OP(S)(OH)O—;

each Q⁷ is independently optionally substituted hydrocarbon or optionally substituted heteroorganic (e.g., C₁₋₆ alkane-triyl, optionally substituted C₁₋₆ alkane-tetrayl, optionally substituted C₂₋₆ heteroalkane-triyl, or optionally substituted C₂₋₆ heteroalkane-tetrayl); and

each R^{a} is independently H or an amino acid side chain; provided that at least one of Q³, Q⁴, and Q⁵ is present.

In some instances, for each occurrence of [-Q³-Q⁴-Q⁵]ₛ-, at least one of Q³, Q⁴, and Q⁵ is present.

In some instances, Q⁷ may be a structure selected from the group consisting of:

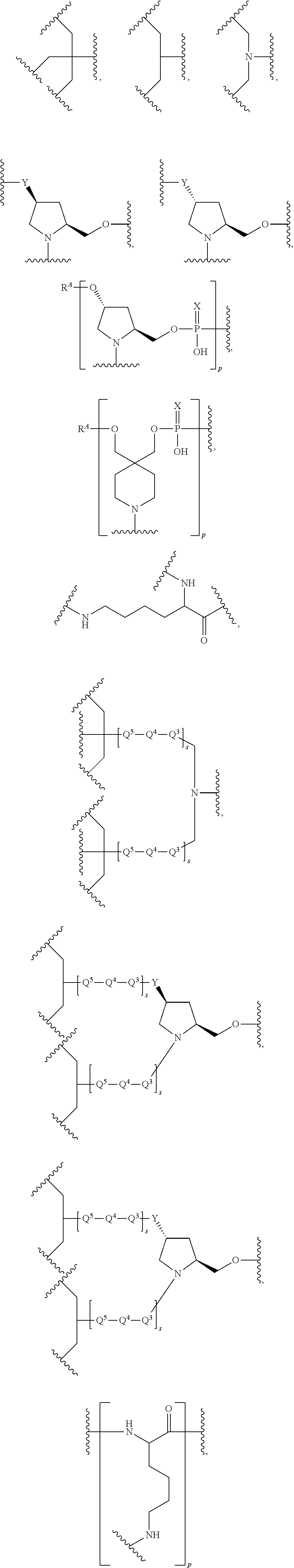

where RA is H or oligonucleotide, X is O or S, Y is O or NH, and the remaining variables are as described for formula (I).

Group -LinkA- may include a poly(alkylene oxide) (e.g., polyethylene oxide, polypropylene oxide, poly(trimethylene oxide), polybutylene oxide, poly(tetramethylene oxide), and diblock or triblock co-polymers thereof). In some embodiments, -LinkA- includes polyethylene oxide (e.g., poly(ethylene oxide) having a molecular weight of less than 1 kDa).

In some instances, -LinkA(-T)ₚ is of the following structure:

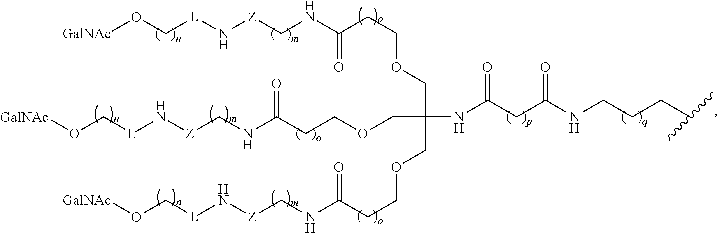

where each L is independently CO or CH₂, each Z is independently CO or CH₂, each n is independently 1 to 9, each m is independently 1 to 5, each o is independently 0 to 1, each p is independently 1 to 10, and each q is independently 1 to 10.

In some instances, each L is CH₂. In some instances, each Z is CO. In some instances, each n is 5. In some instances, each m is 2. In some instances, each o is 1. In some instances, each p is 2. In some instances, each p is 3. In some instances, each q is 4.

In some instances, -LinkA(-T)ₚ is of the following structure:

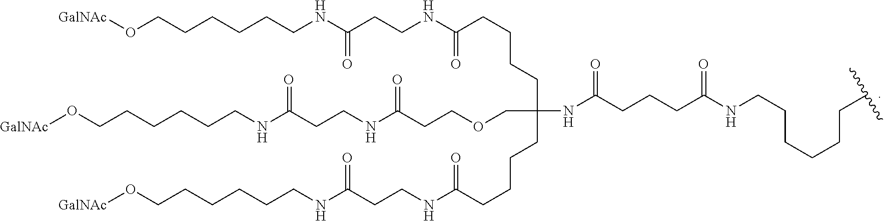

In some instances, -LinkA(-T)ₚ is covalently bonded to a phosphate that is bonded to a 5′-terminal nucleoside. In some instances, -LinkA(-T)ₚ is covalently bonded to phosphate that is bonded to a 3′-terminal nucleoside.

In some instances, -Q²([-Q³-Q⁴-Q₅]ₛ-Q⁶-T)ₚ is a group of the following structure:

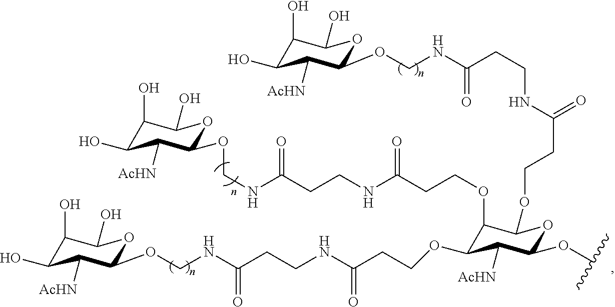

where n is 1 to 20 (e.g., 6).

In some instances, -Q²([-Q³-Q⁴-Q⁵]ₛ-Q⁶-T)ₚ is a group of the following structure:

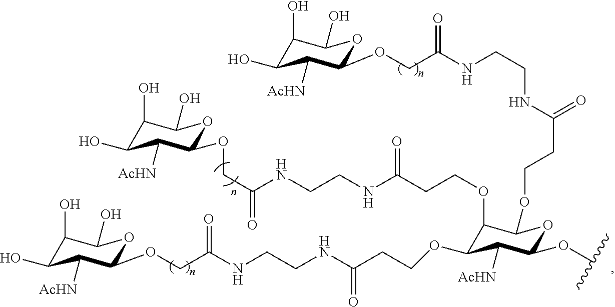

where n is 1 to 20 (e.g., 6).

In some instances, -LinkA(-T)ₚ is a group of the following structure:

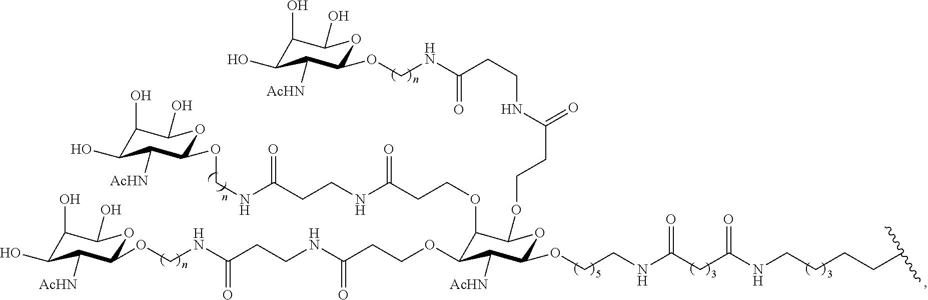

where n is 1 to 20.

In some instances, -LinkA(-T)ₚ is a group of the following structure:

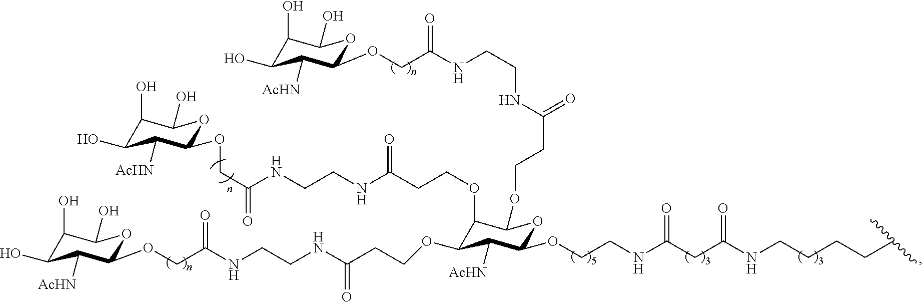

where n is 1 to 20.

In some instances, -LinkA(-T)ₚ is a group of the following structure:

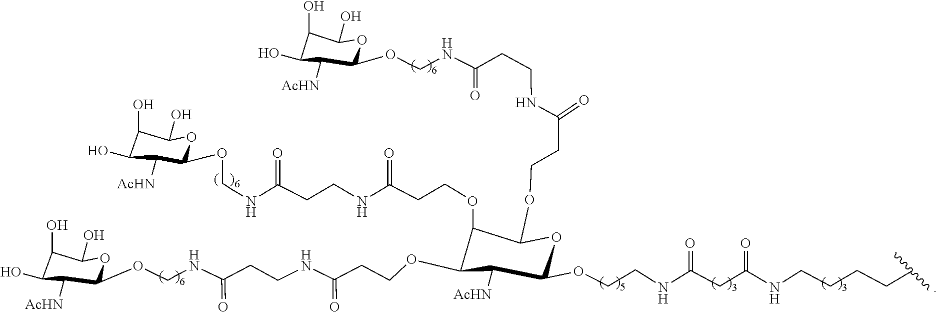

In some instances, -LinkA(-T)ₚ is a group of the following structure:

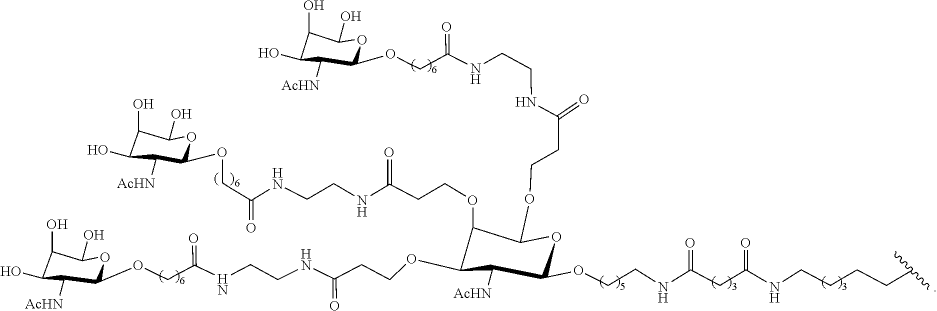

### Hydrophobic Moieties

Advantageously, an oligonucleotide including a hydrophobic moiety may exhibit superior cellular uptake, as compared to an oligonucleotide lacking the hydrophobic moiety. Oligonucleotides including a hydrophobic moiety may therefore be used in compositions that are substantially free of transfecting agents. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently linked to the oligonucleotide backbone (e.g., 5′-terminus). Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be an optionally substituted C₁₋₆₀ hydrocarbon (e.g., optionally substituted C₁₋₆₀ alkylene) or an optionally substituted C₂₋₆₀ heteroorganic (e.g., optionally substituted C₂₋₆₀ heteroalkylene), where the linker may be optionally interrupted with one, two, or three instances independently selected from the group consisting of an optionally substituted arylene, optionally substituted heterocyclylene, and optionally substituted cycloalkylene. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

### Cell Penetrating Peptides

One or more cell penetrating peptides (e.g., from 1 to 6 or from 1 to 3) can be attached to an oligonucleotide disclosed herein as an auxiliary moiety. The CPP can be linked to the oligonucleotide through a disulfide linkage, as disclosed herein. Thus, upon delivery to a cell, the CPP can be cleaved intracellularly, e.g., by an intracellular enzyme (e.g., protein disulfide isomerase, thioredoxin, or a thioesterase) and thereby release the polynucleotide.

CPPs are known in the art (e.g., TAT or Arg₈) (Snyder and Dowdy, 2005, Expert Opin. Drug Deliv. 2, 43-51). Specific examples of CPPs including moieties suitable for conjugation to the oligonucleotides disclosed herein are provided, e.g., in WO 2015/188197; the disclosure of these CPPs is incorporated by reference herein.

CPPs are positively charged peptides that are capable of facilitating the delivery of biological cargo to a cell. It is believed that the cationic charge of the CPPs is essential for their function. Moreover, the transduction of these proteins does not appear to be affected by cell type, and these proteins can efficiently transduce nearly all cells in culture with no apparent toxicity. In addition to full-length proteins, CPPs have also been used successfully to induce the intracellular uptake of DNA, antisense polynucleotides, small molecules, and even inorganic 40 nm iron particles suggesting that there is considerable flexibility in particle size in this process.

In one embodiment, a CPP useful in the methods and compositions of the invention includes a peptide featuring substantial alpha-helicity. It has been discovered that transfection is optimized when the CPP exhibits significant alpha-helicity. In another embodiment, the CPP includes a sequence containing basic amino acid residues that are substantially aligned along at least one face of the peptide. A CPP useful in the invention may be a naturally occurring peptide or a synthetic peptide.

### Polymers

An oligonucleotide of the invention may include covalently attached neutral polymer-based auxiliary moieties. Neutral polymers include poly(C₁₋₆ alkylene oxide), e.g., poly(ethylene glycol) and poly(propylene glycol) and copolymers thereof, e.g., di- and triblock copolymers. Other examples of polymers include esterified poly(acrylic acid), esterified poly(glutamic acid), esterified poly(aspartic acid), poly(vinyl alcohol), poly(ethylene-co-vinyl alcohol), poly(N-vinyl pyrrolidone), poly(ethyloxazoline), poly(alkylacrylates), poly(acrylamide), poly(N-alkylacrylamides), poly(N-acryloylmorpholine), poly(lactic acid), poly(glycolic acid), poly(dioxanone), poly(caprolactone), styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyurethane, N-isopropylacrylamide polymers, and poly(N,N-dialkylacrylamides). Exemplary polymer auxiliary moieties may have molecular weights of less than 100, 300, 500, 1000, or 5000 Da (e.g., greater than 100 Da). Other polymers are known in the art.

### Nucleobase Modifications

Oligonucleotides of the invention may include one or more modified nucleobases. Unmodified nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and 0-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deazaadenine, 7-deazaguanine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The replacement of cytidine with 5-methylcytidine can reduce immunogenicity of oligonucleotides, e.g., those oligonucleotides having CpG units.

The replacement of one or more guanosines with, e.g., 7-deazaguanosine or 6-thioguanosine, may inhibit the antisense activity reducing G tetraplex formation within antisense oligonucleotides.

### Sugar Modifications

Oligonucleotides of the invention may include one or more sugar modifications in nucleosides. Nucleosides having an unmodified sugar include a sugar moiety that is a furanose ring as found in ribonucleosides and 2′-deoxyribonucleosides.

Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-5-2′-substituted ribose), bridged sugars (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

Typically, a sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. A locking modification is an incorporation of a bridge between 4′-carbon atom and 2′-carbon atom of ribofuranose. Nucleosides having a sugar with a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

### Internucleoside Linkage Modifications

Oligonucleotides of the invention may include one or more internucleoside linkage modifications. The two main classes of internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH₃)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Modified linkages, compared to natural phosphodiester linkages, can be used to alter, typically increase, nuclease resistance of the oligonucleotide. Methods of preparation of phosphorous-containing and non-phosphorous-containing internucleoside linkages are known in the art.

Internucleoside linkages may be stereochemically enriched. For example, phosphorothioate-based internucleoside linkages (e.g., phosphorothioate diester or phosphorothioate triester) may be stereochemically enriched. The stereochemically enriched internucleoside linkages including a stereogenic phosphorus are typically designated S_{P} or R_{P} to identify the absolute stereochemistry of the phosphorus atom. Within an oligonucleotide, S_{P} phosphorothioate indicates the following structure:

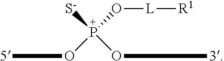

Within an oligonucleotide, R_{P} phosphorothioate indicates the following structure:

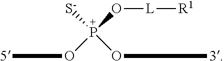

The oligonucleotides of the invention may include one or more neutral internucleoside linkages. Non-limiting examples of neutral internucleoside linkages include phosphotriesters, phosphorothioate triesters, methylphosphonates, methylenemethylimino (5′-CH₂—N(CH₃)—O-3′), amide-3 (5′-CH₂—C(═O)—N(H)-3′), amide-4 (5′-CH₂—N(H)—C(═O)-3′), formacetal (5′-O—CH₂—O-3′), and thioformacetal (5′-S—CH₂—O-3′). Further neutral internucleoside linkages include nonionic linkages including siloxane (dialkylsiloxane), carboxylate ester, carboxamide, sulfide, sulfonate ester, and amides (See for example: Carbohydrate Modifications in Antisense Research; Y. S. Sanghvi and P. D. Cook, Eds., ACS Symposium Series 580; Chapters 3 and 4, 40-65).

### Terminal Modifications

Oligonucleotides of the invention may include a terminal modification, e.g., a 5′-terminal modification or a 3′-terminal modification.

The 5′ end of an oligonucleotide may be, e.g., hydroxyl, a hydrophobic moiety, a targeting moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. An unmodified 5′-terminus is hydroxyl or phosphate. An oligonucleotide having a 5′ terminus other than 5′-hydroxyl or 5′-phosphate has a modified 5′ terminus.

The 3′ end of an oligonucleotide may be, e.g., hydroxyl, a targeting moiety, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An unmodified 3′-terminus is hydroxyl or phosphate. An oligonucleotide having a 3′ terminus other than 3′-hydroxyl or 3′-phosphate has a modified 3′ terminus.

The terminal modification (e.g., 5′-terminal modification) may be, e.g., a targeting moiety as described herein.

The terminal modification (e.g., 5′-terminal modification) may be, e.g., a hydrophobic moiety as described herein.

### Complementarity

In some embodiments, oligonucleotides of the invention are complementary to a LIPA target sequence over the entire length of the oligonucleotide. In other embodiments, oligonucleotides are at least 99%, 95%, 90%, 85%, 80%, or 70% complementary to the LIPA target sequence. In further embodiments, oligonucleotides are at least 80% (e.g., at least 90% or at least 95%) complementary to the LIPA target sequence over the entire length of the oligonucleotide and include a nucleobase sequence that is fully complementary to a LIPA target sequence. The nucleobase sequence that is fully complementary may be, e.g., 6 to 20, 10 to 18, or 18 to 20 contiguous nucleobases in length.

An oligonucleotide of the invention may include one or more (e.g., 1, 2, 3, or 4) mismatched nucleobases relative to the target nucleic acid. In certain embodiments, a splice-switching activity against the target is reduced by such mismatch, but activity against a non-target is reduced by a greater amount. Thus, the off-target selectivity of the oligonucleotides may be improved.

### Methods for Preparing Compositions

The present disclosure provides methods for preparing or generating compositions provided herein. A nucleic acid molecule, such as an oligonucleotide, comprising a targeted sequence may be generated, for example, by various nucleic acid synthesis approaches. For example, a nucleic acid molecule comprising a sequence targeted to a splice site may be generated by oligomerization of modified and/or unmodified nucleosides, thereby producing DNA or RNA oligonucleotides. Antisense oligonucleotides can be prepared, for example, by solid phase synthesis. Such solid phase synthesis can be performed, for example, in multi-well plates using equipment available from vendors such as Applied Biosystems (Foster City, CA). It is well known to use similar techniques to prepare oligonucleotides such as the phosphorothioates and alkylated derivatives. Oligonucleotides may be subjected to purification and/or analysis using methods known to those skilled in the art. For example, analysis methods may include capillary electrophoresis (CE) and electrospray-mass spectroscopy.

### Pharmaceutical Compositions

An oligonucleotide of the invention may be included in a pharmaceutical composition. A pharmaceutical composition typically includes a pharmaceutically acceptable diluent or carrier. A pharmaceutical composition may include (e.g., consist of), e.g., a sterile saline solution and an oligonucleotide of the invention. The sterile saline is typically a pharmaceutical grade saline. A pharmaceutical composition may include (e.g., consist of), e.g., sterile water and an oligonucleotide of the invention. The sterile water is typically a pharmaceutical grade water. A pharmaceutical composition may include (e.g., consist of), e.g., phosphate-buffered saline (PBS) and an oligonucleotide of the invention. The sterile PBS is typically a pharmaceutical grade PBS.

Pharmaceutical compositions may include one or more oligonucleotides and one or more excipients. Excipients may be selected from water, salt solutions, alcohol, polyethylene glycols, gelatin, lactose, amylase, magnesium stearate, talc, silicic acid, viscous paraffin, hydroxymethylcellulose and polyvinylpyrrolidone.

Pharmaceutical compositions including an oligonucleotide encompass any pharmaceutically acceptable salts of the oligonucleotide. Pharmaceutical compositions including an oligonucleotide, upon administration to a subject (e.g., a human), are capable of providing (directly or indirectly) the biologically active metabolite or residue thereof. Accordingly, for example, the disclosure is also drawn to pharmaceutically acceptable salts of oligonucleotides. Suitable pharmaceutically acceptable salts include, but are not limited to, sodium and potassium salts. In certain embodiments, prodrugs include one or more conjugate group(s) attached to an oligonucleotide, wherein the one or more conjugate group(s) is cleaved by endogenous enzymes within the body.

Lipid moieties have been used in nucleic acid therapies in a variety of methods. In certain such methods, the nucleic acid, such as an oligonucleotide, is introduced into preformed liposomes or lipoplexes made of mixtures of cationic lipids and neutral lipids. DNA complexes with mono- or poly-cationic lipids may form, e.g., without the presence of a neutral lipid. A lipid moiety may be, e.g., selected to increase distribution of a pharmaceutical agent to a particular cell or tissue. A lipid moiety may be, e.g., selected to increase distribution of a pharmaceutical agent to fat tissue. A lipid moiety may be, e.g., selected to increase distribution of a pharmaceutical agent to muscle tissue.

Pharmaceutical compositions may include a delivery system. Examples of delivery systems include, but are not limited to, liposomes and emulsions. Certain delivery systems are useful for preparing certain pharmaceutical compositions including those including hydrophobic compounds. Certain organic solvents such as dimethylsulfoxide may be used.

Pharmaceutical compositions may include one or more tissue-specific delivery molecules designed to deliver the one or more pharmaceutical agents of the present invention to specific tissues or cell types. For example, pharmaceutical compositions may include liposomes coated with a targeting moiety as described herein.

Pharmaceutical compositions may include a co-solvent system. Certain co-solvent systems include, e.g., benzyl alcohol, a nonpolar surfactant, a water-miscible organic polymer, and an aqueous phase. Such co-solvent systems may be used, e.g., for hydrophobic compounds. A non-limiting example of a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Pharmaceutical compositions may be prepared for administration by injection or infusion (e.g., intravenous, subcutaneous, intramuscular, intrathecal, intracerebroventricular, etc.). A pharmaceutical composition may include, e.g., a carrier and may be formulated, e.g., in aqueous solution, e.g., water or physiologically compatible buffers, e.g., Hanks's solution, Ringer's solution, or physiological saline buffer. Other ingredients may also be included (e.g., ingredients that aid in solubility or serve as preservatives). Injectable suspensions may be prepared, e.g., using appropriate liquid carriers, suspending agents and the like. Certain pharmaceutical compositions for injection are presented in unit dosage form, e.g., in ampoules or in multi-dose containers. Certain pharmaceutical compositions for injection may be, e.g., suspensions, solutions, or emulsions in oily or aqueous vehicles, and may contain excipients (e.g., suspending, stabilizing and/or dispersing agents). Certain solvents suitable for use in pharmaceutical compositions for injection include, but are not limited to, lipophilic solvents and fatty oils, e.g., sesame oil, synthetic fatty acid esters (e.g., ethyl oleate or triglycerides), and liposomes.

### Methods of the Invention

The invention provides methods of using oligonucleotides of the invention.

A method of the invention may be a method of increasing the level of an exon-containing (e.g., exon 8-containing) LIPA mRNA molecules in a cell expressing an aberrant LIPA gene by contacting the cell with an antisense oligonucleotide of the invention.

A method of the invention may be a method of treating Wolman Disease or Cholesteryl Ester Storage Disease in a subject having an aberrant LIPA gene by administering a therapeutically effective amount of an antisense oligonucleotide of the invention or a pharmaceutical composition of the invention to the subject in need thereof.

The oligonucleotide of the invention or the pharmaceutical composition of the invention may be administered to the subject using methods known in the art. For example, the oligonucleotide of the invention or the pharmaceutical composition of the invention may be administered parenterally (e.g., intravenously, intramuscularly, subcutaneously, transdermally, intranasally, or intrapulmonarily) to the subject.

Dosing is typically dependent on a variety of factors including, e.g., severity and responsiveness of the disease state to be treated. The treatment course may last, e.g., from several days to several years, or until a cure is effected or a diminution of the disease state is achieved. Optimal dosing schedules can be calculated from measurements of drug accumulation in the body of the patient. Thus, optimum dosages, dosing methodologies and repetition rates can be established as needed. Optimum dosages may vary depending on the relative potency of individual oligonucleotides, and can generally be estimated based on EC₅₀s found to be effective in in vitro and in vivo animal models. In general, dosage may be from 0.01 μg to 1 g per kg of body weight, and may be given once or more daily, weekly, monthly, bimonthly, trimonthly, every six months, annually, or biannually. Frequency of dosage may vary. Repetition rates for dosing may be established, for example, based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 1 g per kg of body weight, e.g., once daily, twice daily, three times daily, every other day, weekly, biweekly, monthly, bimonthly, trimonthly, every six months, annually or biannually.

Methods of treating Wolman Disease or Cholesteryl Ester Storage Disease in a subject in need thereof may also include administering to the subject a second therapeutic (e.g., a cholesterol lowering statin or a recombinant lysosomal acid lipase (e.g., sebelipase alfa)). Non-limiting examples of statins include HMG CoA reductase inhibitors, e.g., pravastatin, lovastatin, simvastatin, atorvastatin, fluvastatin, and other statins, e.g., fluindostatin. In some embodiments, the method includes administering to the subject a pharmaceutically acceptable salt of a statin or niacin combination (e.g., pravastatin sodium salt, atorvastatin calcium salt, or lovastatin/niacin). In one example, an oligonucleotide of the invention and a statin or a pharmaceutically acceptable salt thereof are administered together in the same pharmaceutical composition. In another example, an oligonucleotide of the invention and a statin or a pharmaceutically acceptable salt thereof are administered separately at about the same time (e.g., one minute apart or less, or five minutes apart or less). In some embodiments, an oligonucleotide of the invention and a statin or a pharmaceutically acceptable salt thereof are administered separately via the same route of administration (e.g., intravenous injection). In some embodiments, an oligonucleotide of the invention and a statin or a pharmaceutically acceptable salt thereof are administered separately via different routes of administration (e.g., intravenous injection of an oligonucleotide of the invention and oral administration of a statin or a pharmaceutically acceptable salt thereof). In some embodiments, the second therapy is a recombinant lysosomal acid lipase (e.g., sebelipase alfa). Details on administration of the recombinant lysosomal acid lipase (e.g., sebelipase alfa) are described, e.g., in U.S. Pat. No. 10,166,274.

The pharmaceutical composition of the invention may contain a statin, e.g., pravastatin, lovastatin, simvastatin, atorvastatin, or fluvastatin in an amount as normally employed for such statin as exemplified in the 71st edition of the Physician's Desk Reference (PDR). Thus, depending upon the particular statin, it may be employed in amounts within the range from about 0.1 mg to 2000 mg per day in single or divided doses, and preferably from about 0.2 to about 200 mg per day. Most preferably for pravastatin, a daily dosage of 10 to 80 mg may be employed; for lovastatin, a daily dosage of 10 to 80 mg may be employed, for simvastatin a daily dosage of 5 to 80 mg may be employed; for atorvastatin, a daily dosage of 10 to 80 mg may be employed; and for fluvastatin, a daily dosage of 20 to 80 mg may be employed.

In some embodiments, an oligonucleotide of the invention is administered prior to a statin. In further embodiments, an oligonucleotide of the invention is administered within 1 hour of the statin administration (e.g., before, e.g., 15 min, 30 min, or 1 hour before). In some embodiments, an oligonucleotide of the invention is administered within 12 hours of the statin (e.g., before, e.g., 1, 2, 3, 4, 5, 6, 7, 8, 9, 10, 11, or 12 hours before). In certain embodiments, an oligonucleotide of the invention is administered within 24 hours of the statin (e.g., before, e.g., 12 or 24 hours before). In particular embodiments, an oligonucleotide of the invention is administered within 1 week of the statin administration (e.g., before, e.g., 1, 2, 3, 4, 5, or 6 days before). In some embodiments, an oligonucleotide of the invention is administered within 1 month of the statin administration (e.g., before, e.g., 1, 2, 3, or 4 weeks before).

In some embodiments, an oligonucleotide of the invention is administered after a statin. In further embodiments, an oligonucleotide of the invention is administered within 1 hour of the statin administration (e.g., after, e.g., 15 min, 30 min, or 1 hour after). In some embodiments, an oligonucleotide of the invention is administered within 12 hours of the statin administration (e.g., after, e.g., 1, 2, 3, 4, 5, 6, 7, 8, 9, 10, 11, or 12 hours after). In certain embodiments, an oligonucleotide of the invention is administered within 24 hours of the statin administration (e.g., after, e.g., 12 or 24 hours after). In particular embodiments, an oligonucleotide of the invention is administered within 1 week of the statin administration (e.g., after, e.g., 1, 2, 3, 4, 5, or 6 days after). In some embodiments, an oligonucleotide of the invention is administered within 1 month of the statin administration (e.g., after, e.g., 1, 2, 3, or 4 weeks after).

### EXAMPLES

The following materials, methods, and examples are illustrative only and not intended to be limiting.

### Materials and Methods

In general, the practice of the present invention employs, unless otherwise indicated, conventional techniques of chemistry, molecular biology, and recombinant DNA technology. See, e.g., Sambrook, Fritsch and Maniatis, Molecular Cloning: Cold Spring Harbor Laboratory Press (1989) and Current Protocols in Molecular Biology, eds. Ausubel et al., John Wiley & Sons (1992).

Oligonucleotides. All antisense oligonucleotides used were obtained from Integrated DNA Technologies Inc. (USA). All bases in the antisense oligonucleotides were 2′-O-methoxyethyl-modified (MOE) with a full phosphorothioate backbone.

Cell lines. Fibroblast cell lines GM00288, GM00863, GM03111, GM06122, and GM09503 were obtained from the Coriell Institute for Medical Research. Lines GM00288 and GM09503 are from ostensibly healthy individuals. The GM06122 is from a clinically unaffected individual who is heterozygous for the LIPA gene mutation (c.796G>T(p.G266X)). The GM00863 line is from a Wolman Disease patient and is heterozygous for LIPA gene mutations c.290C>G (p.T97R) and c.353G>A (p.G118D). The GM03111 line is from a CESD patient and is heterozygous for LIPA gene mutations c.894G>A and c.967_968delAG (p.S323Lfs*44).

Cell culture. Fibroblast cells were grown in Eagle's Minimal Essential Medium (Gibco) supplemented with 15% Fetal Bovine Serum (Gibco) and 1× Non-Essential Amino Acids Solution (Gibco) in a humidified incubator at 37° C. with 5% CO₂. Upon reaching confluency the cells were passaged by washing with Hanks Buffered Saline Solution followed by dissociation with 0.05% Trypsin-EDTA (Gibco) and plated in 4-fold dilution. HepG2 cells were grown in Dulbecco's Modified Eagle's Medium (Gibco) supplemented with 10% Fetal Bovine Serum (Gibco) in a humidified incubator at 37° C. with 5% CO₂. Upon reaching confluency the cells were passaged by washing with Phosphate-Buffered Saline followed by TrypLE (Gibco) dissociation and plated in a culture flask in 2 to 4-fold dilution.

Transfection of fibroblasts with antisense oligonucleotides. Antisense oligonucleotides were transfected at absolute amounts of 300 pmol of an antisense oligonucleotide per well of a 12-well plate containing approximately 125,000 GM03111 fibroblast cells. For this, 200 μL of Opti-MEM media (Gibco) containing 3 μL of Lipofectamine 2000 (Invitrogen) was transferred to each well of a 12-well tissue culture plate containing 300 pmol antisense oligonucleotides. Antisense oligonucleotide-lipid complexes in the mixture were formed by tilting of the plate followed by incubation for 20 minutes at room temperature. Next, approximately 125,000 fibroblast cells in 800 μL fibroblast media solution were added to the antisense oligonucleotide-lipid complexes and incubated for 48 hours at 37° C. and 5% CO₂.

RNA preparation. RNA was isolated using the RNeasy Mini kit (Qiagen), according to manufacturer's instructions.

RT-PCR analysis. First-strand cDNA synthesis was performed using the High-Capacity cDNA Reverse Transcription Kit (Thermo Fisher), according to manufacturer's instructions. Target-specific fragments were amplified by PCR using the primers SEQ ID NO: 115 (TTCTTCTGTGTGGATTTAATGAGAG) and SEQ ID NO: 116 (AATTCTTGGCACTGCTTCCC). PCR reactions contained 5 μl first-strand cDNA product, 0.4 μM forward primer, 0.4 μM reverse primer, 300 μM of each dNTP, 25 mM Tricine, 7.0% Glycerol (m/v), 1.6% DMSO (m/v), 2 mM MgCl₂, 85 mM NH₄-acetate (pH 8.7), and 1 unit Taq DNA polymerase (FroggaBio) in a total volume of 25 μL. Fragments were amplified by a touchdown PCR program (95° C. for 120 sec; 10 cycles of 95° C. for 20 sec, 68° C. for 30 sec with a decrement of 1° C. per cycle, and 72° C. for 60 sec; followed by 20 cycles of 95° C. for 20 sec, 58° C. for 30 sec, and 72° C. for 60 sec; 72° C. for 180 sec).

Capillary electrophoresis. Samples were analyzed using a LabChip GX Touch Nucleic Acid Analyzer using a DNA 1K Hi Sensitivity LabChip and associated reagents according to manufacturer's recommendations (GE).

LIPA enzyme activity assay. Fibroblasts cultured in 12-well plates with approximately 125,000 cells/well were lysed using 200 μL 1% Triton-X with 1× HALT protease inhibitor (Thermo Fisher). Lysates were passed through a 23-gauge needle 5 times and centrifuged at 14,000 rpm for 10 min at 4° C. Supernatant containing the cell lysates were quantified using the Pierce BCA assay (Thermo Fisher) and samples normalized. 4 μg cell lysate was added to each well of a 96-well plate in quadruplicate. 10 μL of 50 μM Lalistat 2 (Cayman Chemicals) or 10% DMSO was added to each well and the plate was incubated at 37° C. for 10 min. The reaction was started by adding 50 μL of 1 mM 4-methylumbelliferyl oleate (4-MUO; Sigma) to each well and plates were incubated at 37° C. for 1 h. Fluorescence was measured at 340 nm(ex)/450 nm(em) in a BioTEK Synergy Neo 2 plate reader. Enzyme activity was determined by subtracting Lalistat 2-treated samples from DMSO-treated samples.

Viability assay. HepG2 cells were reverse-transfected by adding 50 pmol antisense oligonucleotide into each well of a 96-well plate along with 10 μL Lipofectamine RNAiMAX (Gibco) in Opti-MEM (Gibco) and incubated for 20 min at room temperature. Aproximatly 20,000 HepG2 cells were added to each well. Plates were incubated for 48 h at 37° C. and 5% CO2. Viability determination was performed using the Promega CellTiter Fluor kit, following manufacturer's instructions. The plates were incubated at 37° C. for 1.5 h. Fluorescence (ex: 400 nm, em: 505 nm) was measured using the BioTEK Synergy Neo 2 plate reader.

### Example 1. The Splicing and Enzymatic Activity of LIPA is Disrupted in the c.894G>A Variant and can be Partially Rescued Through the Use of Antisense Oligonucleotides

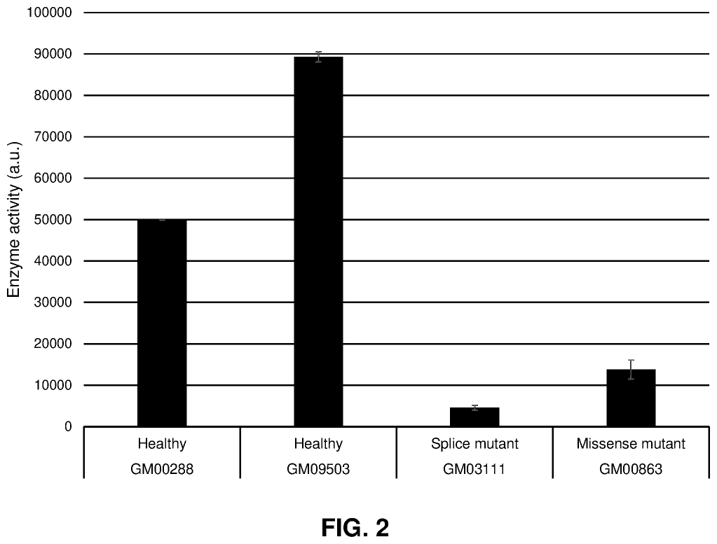

To confirm exon 8 skipping in the c.894G>A variant, RT-PCR was conducted on GM00288, GM03111, GM09503, and GM00863 cells (FIG. 1B). RT-PCR analysis shows that exon 8 is skipped more frequently in cells with the c.894G>A mutation (GM0311). LIPA enzyme activity (FIG. 2) demonstrates reduced LIPA activity in those cell lines where the donor is known to have Wolman Disease or CESD (GM03111 and GM00863).

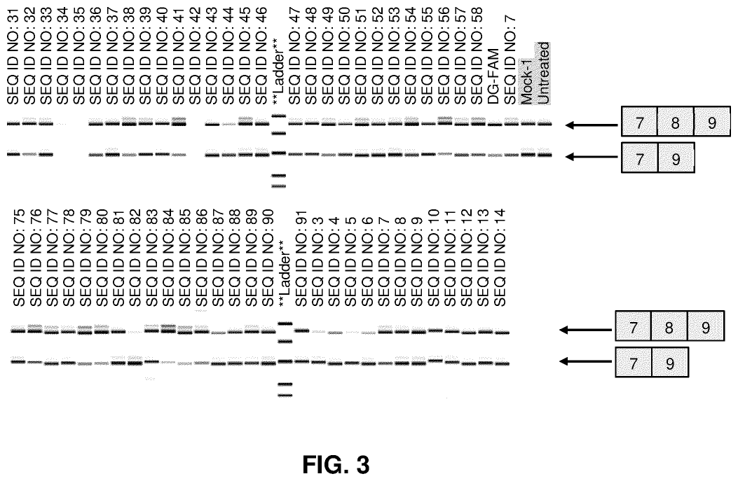

To examine the ability of antisense oligonucleotides to promote exon 8 inclusion in the c.894G>A variant the GM03111 cells were transfected with antisense oligonucleotides having sequences set forth in SEQ ID NOs: 3-91 (see Table 1). FIG. 3 shows representative RT-PCR samples measured by capillary electrophoresis. A 100 bp DNA ladder is shown for size reference with the exon 8 inclusion band at 165 bp and exclusion band at 92 bp. From both FIG. 3 and Table 1 it is clear that targeting the intronic regions surrounding exon 8 induces exon 8 inclusion. Percent spliced in (PSI) for exon 8 was then calculated as well as the change in percent spliced in compared to an inactive control antisense oligonucleotide (dPSI) (Table 1). These observations suggest antisense oligonucleotides targeting the surrounding introns of exon 8 may be useful in the treatment of Wolman Disease or Cholesteryl Ester Storage Disease associated with exon 8 skipping (e.g., Wolman Disease or Cholesteryl Ester Storage Disease caused by the c.894G>A mutation).

Targeting the regions within 100 bp of exon 8 in either of the surrounding introns can lead to a positive dPSI. Targeting these regions (positions 34222-34321 and 34394-34493 in SEQ ID NO: 1; which correspond to chr10:90982339-90982439 and chr10:90982168-90982268, respectively), e.g. using those sequences targeted to be complementary to the pre-mRNA in that region in SEQ ID NOs: 16, 20, 21, 27, 31, 55, 68, 81, 90, and 91 for region 1 and SEQ ID NOs: 3-15, 17-19, 22-26, 28-30, 32-54, 56-67, 69-80, and 82-89 for region 2, may be useful in the treatment of Wolman Disease or Cholesteryl Ester Storage Disease associated with exon 8 skipping (e.g., Wolman Disease or Cholesteryl Ester Storage Disease caused by the c.894G>A mutation). Targeting a “hotspot” corresponding to positions 34398-34480 within region 2, which shows particularly high dPSls, e.g. by using an oligonucleotide having a sequence of SEQ ID NO: 7, 22-26, 32, 34-36, 38-39, 41, 47, 49, 54, 56-59, 63-64, 70-71, 75-77, 79-80, 84-86, 88, or 89, may be particularly useful in the treatment of Wolman Disease or Cholesteryl Ester Storage Disease associated with exon 8 skipping (e.g., Wolman Disease or Cholesteryl Ester Storage Disease caused by the c.894G>A mutation).

TABLE 1

| | | | | | Start on | Stop on | | |
|---|---|---|---|---|---|---|---|---|
| | | | Start Chr10 | End Chr10 | SEQ ID | SEQ ID | | |
| SEQ ID NO: | PSI | Sequence | [hg19/b37] | [hg19/b37] | NO: 1 | NO: 1 | length | dPSI |
| 84 | 0.7852 | CCCAAATGCACTCCTGG | 90982242 | 90982258 | 34403 | 34419 | 17 | 0.5099 |
| 70 | 0.7145 | ATGCACTCCTGGAATG | 90982247 | 90982262 | 34399 | 34414 | 16 | 0.4391 |
| 71 | 0.6913 | CAAATGCACTCCTGGAATG | 90982244 | 90982262 | 34399 | 34417 | 19 | 0.4160 |
| 75 | 0.6730 | TCTATTTGGAAAGGGTTT | 90982186 | 90982203 | 34458 | 34475 | 18 | 0.3977 |
| 22 | 0.6562 | ACCCCAAATGCACTCCTGG | 90982240 | 90982258 | 34403 | 34421 | 19 | 0.3809 |
| 56 | 0.6546 | AACCCCAAATGCACTCCTGG | 90982239 | 90982258 | 34403 | 34422 | 20 | 0.3792 |
| 80 | 0.6459 | AAATGCACTCCTGGAA | 90982245 | 90982260 | 34401 | 34416 | 16 | 0.3706 |
| 79 | 0.6416 | CCAAATGCACTCCTGGA | 90982243 | 90982259 | 34402 | 34418 | 17 | 0.3662 |
| 26 | 0.6392 | CCAAATGCACTCCTGG | 90982243 | 90982258 | 34403 | 34418 | 16 | 0.3638 |
| 85 | 0.6381 | ACCCCAAATGCACTCCTGGA | 90982240 | 90982259 | 34402 | 34421 | 20 | 0.3628 |
| 63 | 0.6267 | CCAAATGCACTCCTGGAATG | 90982243 | 90982262 | 34399 | 34418 | 20 | 0.3514 |
| 88 | 0.6180 | ATGTTGATTTTACATGAA | 90982223 | 90982240 | 34421 | 34438 | 18 | 0.3426 |
| 23 | 0.6154 | CCCAAATGCACTCCTGGA | 90982242 | 90982259 | 34402 | 34419 | 18 | 0.3400 |
| 41 | 0.6127 | CCCCAAATGCACTCCTGG | 90982241 | 90982258 | 34403 | 34420 | 18 | 0.3374 |
| 24 | 0.6071 | CAAATGCACTCCTGGAA | 90982244 | 90982260 | 34401 | 34417 | 17 | 0.3318 |
| 86 | 0.5701 | TCTATTTGGAAAGGGTTTGC | 90982186 | 90982205 | 34456 | 34475 | 20 | 0.2948 |
| 59 | 0.5659 | CCCAAATGCACTCCTGGAA | 90982242 | 90982260 | 34401 | 34419 | 19 | 0.2906 |
| 76 | 0.5484 | CCCCAAATGCACTCCTGGA | 90982241 | 90982259 | 34402 | 34420 | 19 | 0.2730 |
| 38 | 0.5447 | CCAAATGCACTCCTGGAA | 90982243 | 90982260 | 34401 | 34418 | 18 | 0.2694 |
| 49 | 0.5282 | AATGCACTCCTGGAATG | 90982246 | 90982262 | 34399 | 34415 | 17 | 0.2529 |
| 58 | 0.5196 | CAAATGCACTCCTGGA | 90982244 | 90982259 | 34402 | 34417 | 16 | 0.2442 |
| 7 | 0.5122 | CAAATGCACTCCTGGAAT | 90982244 | 90982261 | 34400 | 34417 | 18 | 0.2369 |
| 32 | 0.5042 | AAATGCACTCCTGGAATG | 90982245 | 90982262 | 34399 | 34416 | 18 | 0.2288 |
| 34 | 0.4909 | CCCCAAATGCACTCCTGGAA | 90982241 | 90982260 | 34401 | 34420 | 20 | 0.2155 |
| 54 | 0.4570 | TATTTGGAAAGGGTTTGC | 90982188 | 90982205 | 34456 | 34473 | 18 | 0.1817 |
| 39 | 0.4417 | TTCTATTTGGAAAGGG | 90982185 | 90982200 | 34461 | 34476 | 16 | 0.1664 |
| 25 | 0.4074 | AATGCACTCCTGGAAT | 90982246 | 90982261 | 34400 | 34415 | 16 | 0.1321 |
| 57 | 0.4062 | ACCCCAAATGCACTCCTG | 90982240 | 90982257 | 34404 | 34421 | 18 | 0.1308 |
| 35 | 0.4044 | CCCAAATGCACTCCTGGAAT | 90982242 | 90982261 | 34400 | 34419 | 20 | 0.1290 |
| 89 | 0.4024 | CCAAATGCACTCCTGGAAT | 90982243 | 90982261 | 34400 | 34418 | 19 | 0.1271 |
| 36 | 0.3998 | GTCTTTCTATTTGGAAAGG | 90982181 | 90982199 | 34462 | 34480 | 19 | 0.1245 |
| 77 | 0.3893 | CCCCAAATGCACTCCTG | 90982241 | 90982257 | 34404 | 34420 | 17 | 0.1140 |
| 64 | 0.3840 | AAATGCACTCCTGGAATGC | 90982245 | 90982263 | 34398 | 34416 | 19 | 0.1086 |
| 51 | 0.3600 | CCCAAATGCACTCCTG | 90982242 | 90982257 | 34404 | 34419 | 16 | 0.0847 |
| 47 | 0.3547 | AAATGCACTCCTGGAAT | 90982245 | 90982261 | 34400 | 34416 | 17 | 0.0794 |
| 12 | 0.3501 | TCTGATGTTGATTTTACA | 90982219 | 90982236 | 34425 | 34442 | 18 | 0.0748 |
| 48 | 0.3464 | GCAGGTTGTCTTTCTAT | 90982174 | 90982190 | 34471 | 34487 | 17 | 0.0711 |
| 40 | 0.3393 | GTAAGCAGGTTGTCTTTCTA | 90982170 | 90982189 | 34472 | 34491 | 20 | 0.0640 |
| 78 | 0.3353 | TCTGATGTTGATTTTA | 90982219 | 90982234 | 34427 | 34442 | 16 | 0.0600 |
| 45 | 0.3272 | AACCCCAAATGCACTCCTG | 90982239 | 90982257 | 34404 | 34422 | 19 | 0.0519 |
| 83 | 0.3185 | AATGCACTCCTGGAATGC | 90982246 | 90982263 | 34398 | 34415 | 18 | 0.0432 |
| 62 | 0.3071 | TTCTGATGTTGATTTTA | 90982218 | 90982234 | 34427 | 34443 | 17 | 0.0318 |
| 74 | 0.3064 | TGTTGATTTTACATGAACC | 90982224 | 90982242 | 34419 | 34437 | 19 | 0.0311 |
| 13 | 0.3058 | ACCTTTCTGATGTTGATT | 90982214 | 90982231 | 34430 | 34447 | 18 | 0.0305 |
| 9 | 0.3054 | ACATGAACCCCAAATGCA | 90982234 | 90982251 | 34410 | 34427 | 18 | 0.0301 |
| 15 | 0.3033 | CAGATTTGTAAGCAGG | 90982163 | 90982178 | 34483 | 34498 | 16 | 0.0280 |
| 67 | 0.2930 | ACCCCAAATGCACTCC | 90982240 | 90982255 | 34406 | 34421 | 16 | 0.0176 |
| 29 | 0.2911 | ATGCACTCCTGGAATGC | 90982247 | 90982263 | 34398 | 34414 | 17 | 0.0157 |
| 68 | 0.2895 | ATAATAAACATTGTAT | 90982394 | 90982409 | 34252 | 34267 | 16 | 0.0142 |
| 46 | 0.2891 | TTTGTAAGCAGGTTGTCTTT | 90982167 | 90982186 | 34475 | 34494 | 20 | 0.0137 |
| 37 | 0.2889 | TTGTAAGCAGGTTGTCTT | 90982168 | 90982185 | 34476 | 34493 | 18 | 0.0135 |
| 60 | 0.2884 | GTTGATTTTACATGAACCC | 90982225 | 90982243 | 34418 | 34436 | 19 | 0.0131 |
| 11 | 0.2872 | TGTTGATTTTACATGAAC | 90982224 | 90982241 | 34420 | 34437 | 18 | 0.0119 |
| 72 | 0.2813 | TGCACTCCTGGAATGCC | 90982248 | 90982264 | 34397 | 34413 | 17 | 0.0060 |
| 98 | 0.2809 | AATACATTGAAATGAAGA | 90982350 | 90982367 | 34294 | 34311 | 18 | 0.0055 |
| 14 | 0.2781 | CCCAGACCTTTCTGATGT | 90982209 | 90982226 | 34435 | 34452 | 18 | 0.0027 |
| 10 | 0.2778 | ATTTTACATGAACCCCAA | 90982229 | 90982246 | 34415 | 34432 | 18 | 0.0024 |
| 81 | 0.2755 | ATAATAAACATTGTATTT | 90982394 | 90982411 | 34250 | 34267 | 18 | 0.0002 |
| 65 | 0.2739 | ATGCACTCCTGGAATGCC | 90982247 | 90982264 | 34397 | 34414 | 18 | −0.0014 |
| 18 | 0.2729 | GCACTCCTGGAATGCC | 90982249 | 90982264 | 34397 | 34412 | 16 | −0.0025 |
| 33 | 0.2716 | ACCCCAAATGCACTCCT | 90982240 | 90982256 | 34405 | 34421 | 17 | −0.0037 |
| 90 | 0.2711 | TAATAAACATTGTATTTT | 90982395 | 90982412 | 34249 | 34266 | 18 | −0.0042 |
| 42 | 0.2707 | CAAATGCACTCCTGGAATGC | 90982244 | 90982263 | 34398 | 34417 | 20 | −0.0046 |
| 21 | 0.2694 | TTCAAAGCACTAAAAACT | 90982417 | 90982434 | 34227 | 34244 | 18 | −0.0059 |
| 55 | 0.2694 | AGCACTAAAAACTAGA | 90982422 | 90982437 | 34224 | 34239 | 16 | −0.0059 |
| 50 | 0.2686 | TGCACTCCTGGAATGC | 90982248 | 90982263 | 34398 | 34413 | 16 | −0.0067 |
| 96 | 0.2685 | AATGAAGAATGAAAACAG | 90982360 | 90982377 | 34284 | 34301 | 18 | −0.0068 |
| 27 | 0.2677 | TGATAATAAACATTGTA | 90982392 | 90982408 | 34253 | 34269 | 17 | −0.0076 |
| 94 | 0.2675 | GAAAACAGCATTAAGGTG | 90982370 | 90982387 | 34274 | 34291 | 18 | −0.0078 |
| 95 | 0.2665 | AGAATGAAAACAGCATTA | 90982365 | 90982382 | 34279 | 34296 | 18 | −0.0088 |
| 93 | 0.2636 | CAGCATTAAGGTGGCATT | 90982375 | 90982392 | 34269 | 34286 | 18 | −0.0117 |
| 20 | 0.2616 | GCCCTTCAAAGCACTAAA | 90982413 | 90982430 | 34231 | 34248 | 18 | −0.0137 |
| 16 | 0.2615 | TCAAAGCACTAAAAAC | 90982418 | 90982433 | 34228 | 34243 | 16 | −0.0139 |
| 31 | 0.2605 | TGCCCTTCAAAGCACT | 90982412 | 90982427 | 34234 | 34249 | 16 | −0.0149 |
| 30 | 0.2594 | AAATGCACTCCTGGAATGCC | 90982245 | 90982264 | 34397 | 34416 | 20 | −0.0160 |
| 8 | 0.2593 | AACCCCAAATGCACTCCT | 90982239 | 90982256 | 34405 | 34422 | 18 | −0.0160 |
| 43 | 0.2557 | AATGCACTCCTGGAATGCC | 90982246 | 90982264 | 34397 | 34415 | 19 | −0.0196 |
| 53 | 0.2525 | CCCCAAATGCACTCCT | 90982241 | 90982256 | 34405 | 34420 | 16 | −0.0228 |
| 66 | 0.2462 | GCACTCCTGGAATGCCT | 90982249 | 90982265 | 34396 | 34412 | 17 | −0.0292 |
| 52 | 0.2460 | AACCCCAAATGCACTCC | 90982239 | 90982255 | 34406 | 34422 | 17 | −0.0294 |
| 44 | 0.2396 | TGCACTCCTGGAATGCCT | 90982248 | 90982265 | 34396 | 34413 | 18 | −0.0357 |
| 19 | 0.2387 | ATGCACTCCTGGAATGCCT | 90982247 | 90982265 | 34396 | 34414 | 19 | −0.0367 |
| 17 | 0.2357 | AACCCCAAATGCACTC | 90982239 | 90982254 | 34407 | 34422 | 16 | −0.0396 |
| 97 | 0.2347 | ATTGAAATGAAGAATGAA | 90982355 | 90982372 | 34289 | 34306 | 18 | −0.0407 |
| 73 | 0.2325 | AATGCACTCCTGGAATGCCT | 90982246 | 90982265 | 34396 | 34415 | 20 | −0.0429 |
| 99 | 0.2197 | AAATAAATACATTGAAAT | 90982345 | 90982362 | 34299 | 34316 | 18 | −0.0556 |
| 92 | 0.1946 | TAAGGTGGCATTGATAAT | 90982381 | 90982398 | 34263 | 34280 | 18 | −0.0807 |
| 87 | 0.1649 | ATGCACTCCTGGAATGCCTA | 90982247 | 90982266 | 34395 | 34414 | 20 | −0.1104 |
| 69 | 0.1344 | TGCACTCCTGGAATGCCTA | 90982248 | 90982266 | 34395 | 34413 | 19 | −0.1409 |
| 100 | 0.1325 | CTGCAAAATAAATACATT | 90982340 | 90982357 | 34304 | 34321 | 18 | −0.1429 |
| 6 | 0.0909 | GCACTCCTGGAATGCCTA | 90982249 | 90982266 | 34395 | 34412 | 18 | −0.1845 |
| 4 | 0.0827 | AATGCCTACTTGGCTCCA | 90982259 | 90982276 | 34385 | 34402 | 18 | −0.1926 |
| 28 | 0.0753 | CACTCCTGGAATGCCT | 90982250 | 90982265 | 34396 | 34411 | 16 | −0.2000 |
| 113 | 0.0682 | CCAGTGTAACATGTTTTG | 90982274 | 90982291 | 34370 | 34387 | 18 | −0.2071 |
| 5 | 0.0469 | CCTGGAATGCCTACTTGG | 90982254 | 90982271 | 34390 | 34407 | 18 | −0.2284 |
| 101 | 0.0457 | CTAGACTGCAAAATAAAT | 90982335 | 90982352 | 34309 | 34326 | 18 | −0.2296 |
| 3 | 0.0422 | CTACTTGGCTCCAGTGTA | 90982264 | 90982281 | 34380 | 34397 | 18 | −0.2331 |
| 91 | 0.0418 | TTGATAATAAACATTGTA | 90982391 | 90982408 | 34253 | 34270 | 18 | −0.2336 |
| 112 | 0.0247 | GTAACATGTTTTGCACAG | 90982279 | 90982296 | 34365 | 34382 | 18 | −0.2506 |
| 61 | 0.0224 | ACTCCTGGAATGCCTA | 90982251 | 90982266 | 34395 | 34410 | 16 | −0.2530 |
| 111 | 0.0215 | ATGTTTTGCACAGAAGTT | 90982284 | 90982301 | 34360 | 34377 | 18 | −0.2538 |
| 104 | 0.0189 | GTATATACATCCACTCTA | 90982320 | 90982337 | 34324 | 34341 | 18 | −0.2564 |
| 105 | 0.0183 | GTGTTGTATATACATCCA | 90982315 | 90982332 | 34329 | 34346 | 18 | −0.2570 |
| 82 | 0.0158 | CACTCCTGGAATGCCTA | 90982250 | 90982266 | 34395 | 34411 | 17 | −0.2595 |
| 110 | 0.0117 | TTGCACAGAAGTTCCAGC | 90982289 | 90982306 | 34355 | 34372 | 18 | −0.2636 |
| 106 | 0.0073 | AGAATGTGTTGTATATAC | 90982310 | 90982327 | 34334 | 34351 | 18 | −0.2680 |
| 109 | 0.0067 | AGAAGTTCCAGCAGGAGA | 90982295 | 90982312 | 34349 | 34366 | 18 | −0.2686 |
| 102 | 0.0051 | CCACTCTAGACTGCAAAA | 90982330 | 90982347 | 34314 | 34331 | 18 | −0.2703 |
| 103 | 0.0032 | TACATCCACTCTAGACTG | 90982325 | 90982342 | 34319 | 34336 | 18 | −0.2721 |
| 107 | 0.0015 | GCAGGAGAATGTGTTGTA | 90982305 | 90982322 | 34339 | 34356 | 18 | −0.2738 |
| 108 | 0.0000 | TTCCAGCAGGAGAATGTG | 90982300 | 90982317 | 34344 | 34361 | 18 | −0.2753 |
| 114 | 0.0000 | TGGCTCCAGTGTAACATG | 90982269 | 90982286 | 34375 | 34392 | 18 | −0.2753 |

### Example 2 Characterization of Target Regions (Hot Spots) for Increasing Inclusion of LIPA Exon 8

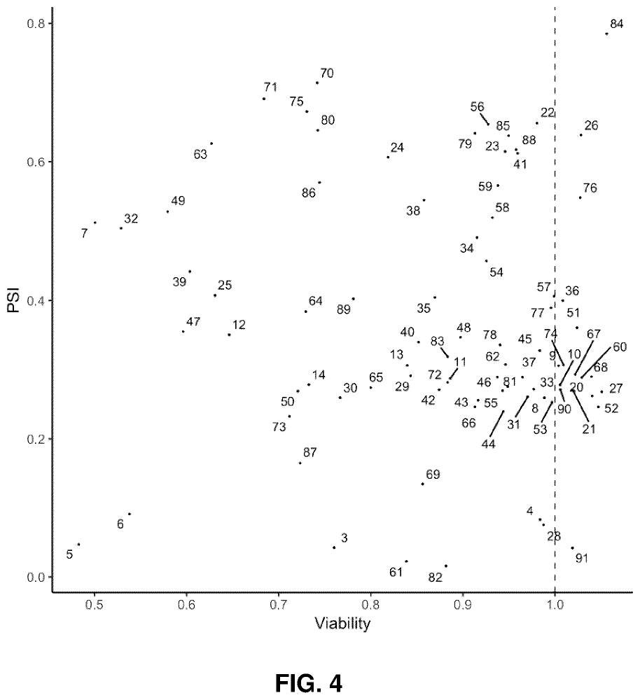

To determine the potential patient tolerability of the screened antisense oligonucleotides, HepG2 (human liver cancer) cells were transfected with antisense oligonucleotides having the sequences set forth in Table 2. Viability of the cells was measured with values normalized to a negative control. A low viability score translates to some observed toxicity in vitro. These values were plotted against their respective PSI in FIG. 4.

These observations suggest antisense oligonucleotides targeting the surrounding introns of exon 8 may be effective in the treatment of Wolman Disease or Cholesteryl Ester Storage Disease associated with exon 8 skipping (e.g., Wolman Disease or Cholesteryl Ester Storage Disease caused by the c.894G>A mutation).

These observations also suggest certain antisense oligonucleotides targeting the surrounding introns of exon 8, namely SEQ ID NOs: 84, 26, 22, 85, 76, 41, 56, 23, 79, 59, 58, 34, and 54 (corresponding to positions 34401-34422 and 34456-34473 in SEQ ID NO: 1; which correspond to chr10: 90982239-90982260 and chr10: 90982188-90982205, respectively) may be particularly effective for use in the treatment of Wolman Disease or Cholesteryl Ester Storage Disease associated with exon 8 skipping (e.g., Wolman Disease or Cholesteryl Ester Storage Disease caused by the c.894G>A mutation).

TABLE 2

| SEQ ID NO | PSI | Viability |
|---|---|---|
| 84 | 0.7852 | 1.056 |
| 70 | 0.7145 | 0.7418 |
| 71 | 0.6913 | 0.6839 |
| 75 | 0.6730 | 0.7306 |
| 22 | 0.6562 | 0.9804 |
| 56 | 0.6546 | 0.9275 |
| 80 | 0.6459 | 0.7425 |
| 79 | 0.6416 | 0.9131 |
| 26 | 0.6392 | 1.0282 |
| 85 | 0.6381 | 0.9497 |
| 63 | 0.6267 | 0.627 |
| 88 | 0.6180 | 0.9578 |
| 23 | 0.6154 | 0.9459 |
| 41 | 0.6127 | 0.9594 |
| 24 | 0.6071 | 0.8187 |
| 86 | 0.5701 | 0.7442 |
| 59 | 0.5659 | 0.938 |
| 76 | 0.5484 | 1.0274 |
| 38 | 0.5447 | 0.8577 |
| 49 | 0.5282 | 0.5794 |
| 58 | 0.5196 | 0.932 |
| 7 | 0.5122 | 0.5005 |
| 32 | 0.5042 | 0.5288 |
| 34 | 0.4909 | 0.9153 |
| 54 | 0.4570 | 0.9255 |
| 39 | 0.4417 | 0.6035 |
| 25 | 0.4074 | 0.6308 |
| 57 | 0.4062 | 0.9989 |
| 35 | 0.4044 | 0.8695 |
| 89 | 0.4024 | 0.781 |
| 36 | 0.3998 | 1.0084 |
| 77 | 0.3893 | 0.9956 |
| 64 | 0.3840 | 0.7294 |
| 51 | 0.3600 | 1.0239 |
| 47 | 0.3547 | 0.5962 |
| 12 | 0.3501 | 0.6463 |
| 48 | 0.3464 | 0.8973 |
| 40 | 0.3393 | 0.8518 |
| 78 | 0.3353 | 0.9404 |
| 45 | 0.3272 | 0.9834 |
| 83 | 0.3185 | 0.8834 |
| 62 | 0.3071 | 0.9463 |
| 74 | 0.3064 | 1.0092 |
| 13 | 0.3058 | 0.8395 |
| 9 | 0.3054 | 1.0039 |
| 67 | 0.2930 | 1.022 |
| 29 | 0.2911 | 0.8432 |
| 68 | 0.2895 | 1.0396 |
| 46 | 0.2891 | 0.9373 |
| 37 | 0.2889 | 0.9647 |
| 60 | 0.2884 | 1.029 |
| 11 | 0.2872 | 0.8863 |
| 72 | 0.2813 | 0.8834 |
| 14 | 0.2781 | 0.7326 |
| 10 | 0.2778 | 1.0056 |
| 81 | 0.2755 | 0.9486 |
| 65 | 0.2739 | 0.7999 |
| 33 | 0.2716 | 0.9769 |
| 90 | 0.2711 | 1.0058 |
| 42 | 0.2707 | 0.8742 |
| 21 | 0.2694 | 1.0196 |
| 55 | 0.2694 | 0.943 |
| 50 | 0.2686 | 0.7207 |
| 27 | 0.2677 | 1.0507 |
| 20 | 0.2616 | 1.0403 |
| 31 | 0.2605 | 0.9703 |
| 30 | 0.2594 | 0.7667 |
| 8 | 0.2593 | 0.9885 |
| 43 | 0.2557 | 0.9165 |
| 53 | 0.2525 | 0.9968 |
| 66 | 0.2462 | 0.913 |
| 52 | 0.2460 | 1.0471 |
| 44 | 0.2396 | 0.9439 |
| 73 | 0.2325 | 0.7117 |
| 87 | 0.1649 | 0.7232 |
| 69 | 0.1344 | 0.8563 |
| 6 | 0.0909 | 0.538 |
| 4 | 0.0827 | 0.9837 |
| 28 | 0.0753 | 0.9876 |
| 5 | 0.0469 | 0.4829 |
| 3 | 0.0422 | 0.7602 |
| 91 | 0.0418 | 1.019 |
| 61 | 0.0224 | 0.8386 |
| 82 | 0.0158 | 0.8816 |

### Example 3 Treatment of Cholesteryl Ester Storage Disease Patient Derived Cells Containing the c.894G>A Variant with a Splice Modulating Antisense Oligonucleotide Increases LIPA Activity

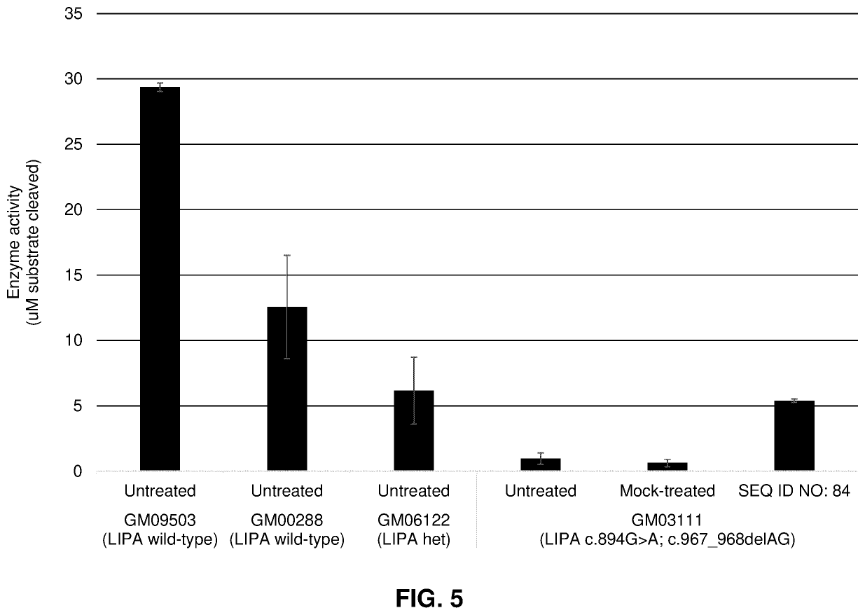

To test whether the rescue of the splice aberration by antisense oligonucleotide treatment would result in a corresponding rescue of function, a LIPA enzyme assay was conducted using GM03111 (LIPA c.894G>A compound heterozygous) fibroblasts transfected with an antisense oligonucleotide shown to increase exon 8 inclusion (FIG. 5), SEQ ID NO:84. Comparing LIPA enzyme activity in GM03111 fibroblasts, with and without antisense oligonucleotide treatment, to samples from an apparently healthy donor's fibroblasts (GM09503, GM00288) or fibroblasts from a clinically unaffected donor with a LIPA heterozygous mutation (GM06122), showed that treated cell activity was rescued to the level of a clinically unaffected individual and 50% of wild-type activity (GM00288). This corresponded to a 10-fold increase in activity versus untreated fibroblasts, indicating that antisense treatment with splice switching oligonucleotides may be effective in the treatment of Wolman Disease or Cholesteryl Ester Storage Disease by partially restoring LIPA activity associated with exon 8 inclusion (e.g., Wolman Disease or Cholesteryl Ester Storage Disease caused by the c.894G>A mutation).

### Example 4. Toxicology Study of Antisense Oligonucleotides Including a Targeting Moiety

The objective of the study was to determine the toxicity of 7 different GaINAc-conjugated oligonucleotides when given as a single subcutaneous injection to CD-1 mice and to assess the persistence, delayed onset or reversibility of any changes during a 7-day postdose period. The test items each contained an oligonucleotide sequence conjugated to an N-acetylgalactosamine (GaINAc) cluster show below:

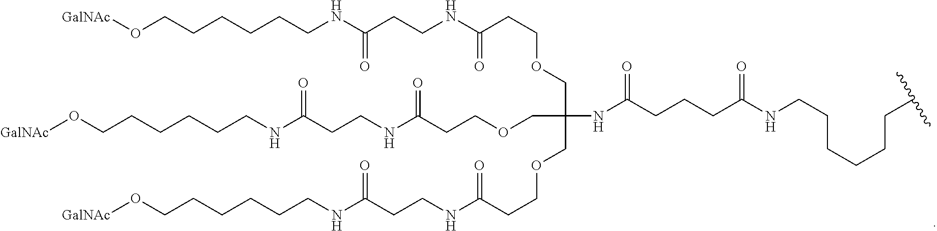

The test and control/vehicle items were administered on one occasion by subcutaneous injection as shown in Table 3.

TABLE 3

| | | Dose | Dose | Dose | No. of | |
|---|---|---|---|---|---|---|
| Group | | Level | concentration | Volume | Animals | |
| No. | Treatment^{a} | (mg/kg)^{b} | (mg/mL)^{b} | (mL/kg) | Males | Females |
| 1 | Vehicle^{c} | 0 | 0 | 5 | 3 | 3 |
| 2 | 22 | 30 | 6 | 5 | 3 | 3 |
| 3 | 22 | 100 | 20 | 5 | 3 | 3 |
| 4 | 22 | 300 | 60 | 5 | 3 | 3 |
| 5 | 23 | 30 | 6 | 5 | 3 | 3 |
| 6 | 23 | 100 | 20 | 5 | 3 | 3 |
| 7 | 23 | 300 | 60 | 5 | 3 | 3 |
| 8 | 26 | 30 | 6 | 5 | 3 | 3 |
| 9 | 26 | 100 | 20 | 5 | 3 | 3 |
| 10 | 26 | 300 | 60 | 5 | 3 | 3 |
| 11 | 41 | 30 | 6 | 5 | 3 | 3 |
| 12 | 41 | 100 | 20 | 5 | 3 | 3 |
| 13 | 41 | 300 | 60 | 5 | 3 | 3 |
| 14 | 56 | 30 | 6 | 5 | 3 | 3 |
| 15 | 56 | 100 | 20 | 5 | 3 | 3 |
| 16 | 56 | 300 | 60 | 5 | 3 | 3 |
| 17 | 84 | 30 | 6 | 5 | 3 | 3 |
| 18 | 84 | 100 | 20 | 5 | 3 | 3 |
| 19 | 84 | 300 | 60 | 5 | 3 | 3 |
| 20 | 85 | 30 | 6 | 5 | 3 | 3 |
| 21 | 85 | 100 | 20 | 5 | 3 | 3 |
| 22 | 85 | 300 | 60 | 5 | 3 | 3 |

^{a}The treatment column lists either Vehicle or a SEQ ID NO of the administered antisense oligonucleotide conjugated to the GalNAc cluster shown above.
^{b}Dose levels and concentrations were expressed as the full weight of the oligonucleotide/GalNAc conjugate.
^{c}The control animals were administered phosphate-buffered saline (PBS) alone.

Protocol. The test oligonucleotides were prepared fresh on the day of dosing. The vials of preweighed test oligonucleotides were removed from the freezer (−20±10° C.) and allowed to equilibrate for 30 minutes at room temperature. Once equilibrated, 2 mL of Phosphate Buffered Saline (PBS) were added to each vial and the formulation was mixed by gentle inversion and filtered through a 0.22 pm PVDF filter into a sterile container. The formulations were kept at room temperature pending transfer to the animal rooms for dosing.

Sixty-six male and female CD-1 mice were received from Charles River Laboratories Inc. (Raleigh, NC). On the first treatment day (Day 1) mice weighed 19.6 g to 37.9 g. Animals were assigned to their dose levels by block randomization based on body weights.

All animals were cared for, fed, watered and housed in accordance with ITR SOPs and codes of practice for animal welfare.

The test oligonucleotides and control/vehicle items were administered by subcutaneous injection on Day 1 at a dose level of 5 mL/kg per animal. The actual volume administered to each mouse was calculated and adjusted based on the most recent practical body weight of each animal. The difference between the weight of the test and control items containers before and after dosing, and the theoretical volume of dose formulations to be administered to the animals revealed that all animals received 101% to 104% of their nominal dose.

During the in-life period, mortality and clinical signs were monitored daily; the detailed clinical examinations and bodyweight were performed on Day 1 and prior to necropsy on Day 8. Food consumption was recorded on Day 1 and Day 7.

The maximum amount of blood was collected from each mouse via the abdominal aorta or cardiac puncture under isoflurane anesthesia and was transferred to the Clinical Pathology department where various clinical chemistry parameters were measured.

All animals were subjected to a gross pathology which consisted of an external examination, including identification of all clinically-recorded lesions, as well as a detailed internal examination. On completion of the gross examination, select organs were weighed, and all livers, kidneys and gross lesions were examined for microscopic findings.

Results. There were no clinical signs that could be attributed to the single subcutaneous administration of GaINAc conjugates of SEQ ID NOS: 22, 23, 26, 41, 56, 84, or 85 at dose levels up to 300 mg/kg. The clinical signs that were noted (including eating like behaviour, eye opacity, closed eyes, swelling anus, kinked tail, and crusts on the pinna) were considered to be incidental and/or procedurally related as they were observed at low incidence and/or were also noted during the pre-treatment period.

Similar to the absence of clinical signs there were no changes in body weights that could be attributed to the single subcutaneous administration of GaINAc conjugates of SEQ ID NOS: 22, 23, 26, 41, 56, 84, or 85 at a concentration of up to 300 mg/kg. The body weight fluctuations that were noted during the study were considered to be within the normal variation range for this species.

As with body weights, no changes in food consumption that could be attributed to the single subcutaneous administration of GaINAc conjugates of SEQ ID NOS: 22, 23, 26, 41, 56, 84, or 85 at dose levels up to 300 mg/kg. The food consumption fluctuations that were noted during the study were considered to be within the normal variation range for this species.

There were no changes in clinical chemistry parameters that could be attributed to the single subcutaneous administration of GaINAc conjugates of SEQ ID NOS: 22, 23, 26, 41, 56, 84, or 85 at dose levels up to 300 mg/kg. The observed values are comparable to the Control/Vehicle group and all fluctuations that were noted during the study were considered to be within the normal variation range for this species.

There were no changes in organ weights that could be attributed to the single subcutaneous administration of GaINAc conjugates of SEQ ID NOS: 22, 23, 26, 41, 56, 84, or 85 at dose levels of up to 300 mg/kg. For some animals the organ weights of some organs were higher or lower when compared to the Control/Vehicle animals; however, the weights were uncorrelated with the other animals in the group and were therefore considered to be aberrant. It must be noted that in females exhibiting estrus, the uterus weight was excluded from the group calculations.

There were no macroscopic findings in male and female mice related to the treatment of any of the seven test items: GaINAc conjugates of SEQ ID NOS: 22, 23, 26, 41, 56, 84, or 85 at doses of 30, 100, and 300 mg/kg. Findings noted infrequently at the dosing site of some treatment groups were considered procedure-related.

There were no microscopic findings in the liver and kidney of male mice related to treatment with any of the seven test items: GaINAc conjugates of SEQ ID NOS: 22, 23, 26, 41, 56, 84, or 85 at doses of 30, 100, and 300 mg/kg.

There were no microscopic findings in the liver and kidney of female mice related to treatment with two of the test items: GaINAc conjugates of SEQ ID NOS: 23 or 84 at doses of 30, 100, and 300 mg/kg.

Minimal tubular basophilic granulation was noted occasionally in the kidney of female mice treated with five of the test items: GaINAc conjugates of SEQ ID NOS: 22, 26, 41, 56, or 85 at doses of 100 and/or 300 mg/kg. There were no microscopic findings in the liver and kidney of female mice related to treatment with these five test items at a dose of 30 mg/kg.

SEQ ID NO: 22: Minimal tubular basophilic granulation was noted in the kidney of one female mouse (⅓) dosed with 300 mg/kg of the SEQ ID NO: 22 conjugated to the GaINAc cluster (Group 4).

SEQ ID NO: 26: Minimal tubular basophilic granulation was noted in the kidney of one female mouse (⅓) dosed with 300 mg/kg of the SEQ ID NO: 26 conjugated to the GaINAc cluster (Group 10).

SEQ ID NO: 41: Minimal tubular basophilic granulation was noted in the kidney of one female mouse (⅓) dosed with 100 mg/kg of DG2455 (Group 12) and one female mouse (⅓) dosed with 300 mg/kg of the SEQ ID NO: 41 conjugated to the GaINAc cluster (Group 13).

SEQ ID NO: 56: Minimal tubular basophilic granulation was noted in the kidney of one female mouse (⅓) dosed with 300 mg/kg of the SEQ ID NO: 56 conjugated to the GaINAc cluster (Group 16).

SEQ ID NO: 85: Minimal tubular basophilic granulation was noted in the kidney of one female mouse (⅓) dosed with 100 mg/kg of DG2499 (Group 21) and two female mice (⅔) dosed with 300 mg/kg of the SEQ ID NO: 85 conjugated to the GaINAc cluster (Group 22).

Findings noted infrequently at the dosing site of some treatment groups were considered procedure-related. Increased hepatocyte rarefaction was noted rarely and sporadically in the liver of female mice from some treatment groups and was considered to be within the range of expected background change.

All other microscopic findings were considered to be incidental as they were not dose-related, of low incidence or severity, and/or considered to be within the expected range of spontaneous background findings.

### Example 5. Preparation of an Exemplary Targeting Moiety

Some targeting moieties may be prepared using techniques and methods known in the art and those described herein. For example, a targeting moiety may be prepared according to the procedure illustrated in Schemes 1, 2, and 3 and described herein.

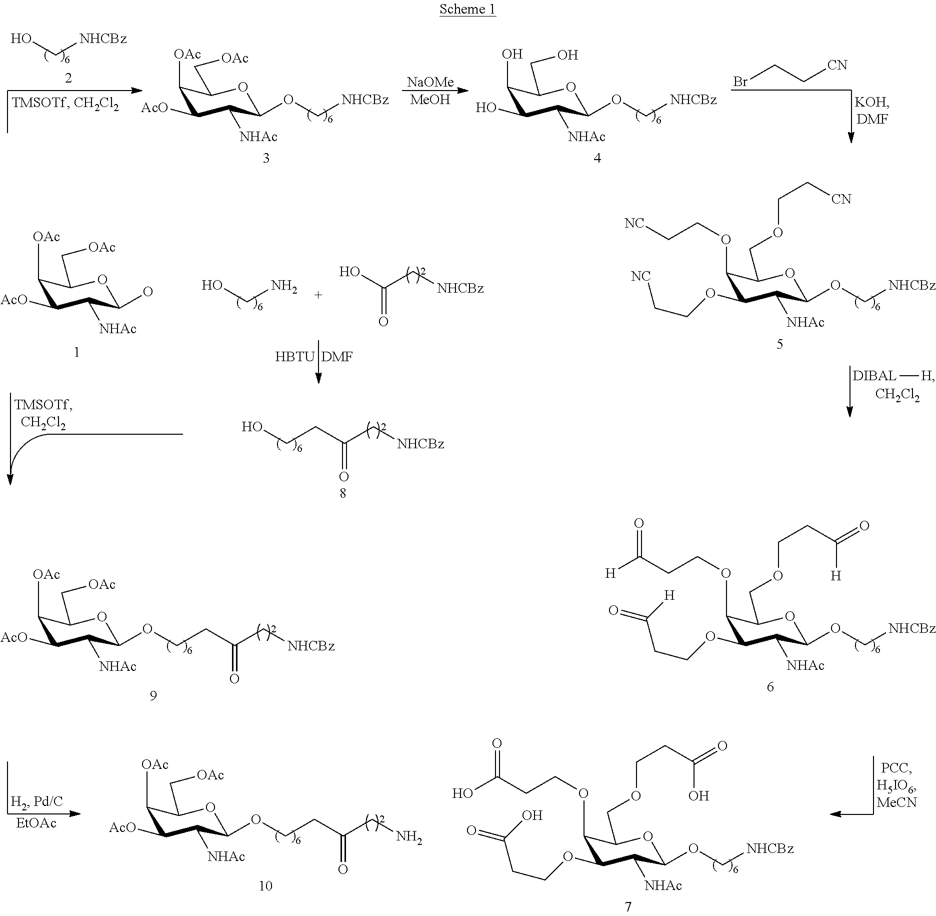

Preparation of compound 3: compound 1 was dissolved in DCM with compound 2 (0.9 equiv.). TMSOTf (1.0 equiv.) was added dropwise at room temperature, and the resulting mixture was stirred for 16 hours. Then, the reaction mixture was washed with 5% aqueous NaHCO3, stirred for 30 minutes, and separated, and the organic phase was collected. The organic phase was then extracted with dichloromethane (DCM) and concentrated to dryness. The product was recrystallized from 2:1 EtOAc/hexane to yield a white solid (83% yield).

Preparation of compound 4: compound 3 was dissolved in 1:1 methanol:CH₂Cl₂. NaOMe (0.11 equiv.) was added, and the resulting mixture was stirred under nitrogen for one hour at room temperature. The reaction mixture was concentration in vacuo to produce a while solid, which was used in the next step without further purification.

Preparation of compound 5: under inert atmosphere, 3-bromopropionitrile (12.1 equiv.) was added dropwise at 0° C. to a DMF solution of compound 4 (1.0 equiv.), and KOH (8.1 equiv.). The resulting mixture was gradually warmed to room temperature and stirred overnight. The mixture was then concentrated to give a residue, which was dissolve in EtOAc and washed with brine. The organic layer was dried over Na₂SO₄, concentrated, and subjected to silica gel chromatography (3:2 EtOAc/hexanes) to give the product.

Preparation of compound 6: to a stirred suspension of compound 5 in anhydrous CH₂Cl₂ at −70° C., DIBAL-H (1.0M) in CH₂Cl₂ may be added dropwise. The resulting mixture may then be stirred under inert atmosphere for 2 hours. The reaction mixture may be worked up using Fieser procedure to remove aluminum byproducts. First, the reaction mixture may be diluted with ether and warmed to 0° C. The imine intermediate may be hydrolyzed by slow addition of water. Then, 15% aqueous NaOH may be added, followed by water. The resulting mixture may be warmed to room temperature and stirred for 15 minutes, at which time, anhydrous MgSO₄ may be added. The resulting mixture may be stirred for 15 minutes and filtered through a Celite® pad. The product may be purified by silica gel chromatography (3:2 EtOAc/hexanes).

Preparation of compound 7: periodic acid may be added to MeCN and stirred vigorously for 15 minutes at room temperature. Compound 6 may then be added, followed by pyridinium chlorochromate (PCC) in MeCN in 2 parts. After 3 hours stirring, the reaction mixture may be diluted with EtOAc and washed with brine, NaHCO₃, brine, and dried over Na₂SO₄. The separated organic layer may be concentrated in vacuo to give compound 7. A quantitative yield is expected for this reaction.

Preparation of compound 8: CBz-protected β-alanine (1 equiv.) and HBTU (1.1 equiv.) were dissolved in DMF. The resulting solution was cooled to 0-10° C., and N,N-diisopropyl-N-ethylamine (DIPEA, 1.5 equiv. was added dropwise. The resulting mixture was stirred for at least 30 minutes at 0-10° C. and then cooled to −25° C. 6-amino-1-hexanol (1 equiv.) in DMF was added dropwise. After 4 hours, the reaction was quenched with water, and the resulting mixture was stirred for 1 hour and filtered, and the filter cake was washed with water. A slurry of the cake and water was filtered twice. The filter cake was dried under vacuum at 40° C. until water content was 0.3% or less (76% yield).

Preparation of compound 9: compound 1 (1.1 equiv.) was dissolved in DCM, and the resulting solution was cooled to 5-15° C. TMSOTf (1.2 equiv.) was added, and the resulting mixture was stirred for 2 hours at 5-15° C. Compound 8 (1.0 equiv.) was added to the reaction mixture, and the resulting mixture was stirred for 16 hours as 30-40° C. The reaction mixture was then cooled to 15-25° C.

Water was added, and the mixture was stirred for 10 minutes. Layers were separated, and the organic phase was washed with water twice. The organic layer was concentrated to dryness. The product was recrystallized from 2:1 EtOAc/hexane and filtered, and the filter cake was dried in vacuo to product the white solid (65% yield).

Preparation of compound 10: compound 9 was dissolved in ethyl acetate (EtOAc) under nitrogen, and trifluoroacetic acid (1.5 equiv) and Pd/C (20% (w/w)) were added with stirring. Hydrogen gas (balloon) was added to the reaction at 2 atm, and the resulting mixture was stirred at room temperature for 2 hours. Solid Pd/C was filtered through a pad of Celite®, and the filtrate was concentrated in vacuo to give a crude product, which may be used without purification in the next step (coupling to the tri-perfluorophenyl ester of compound 7).

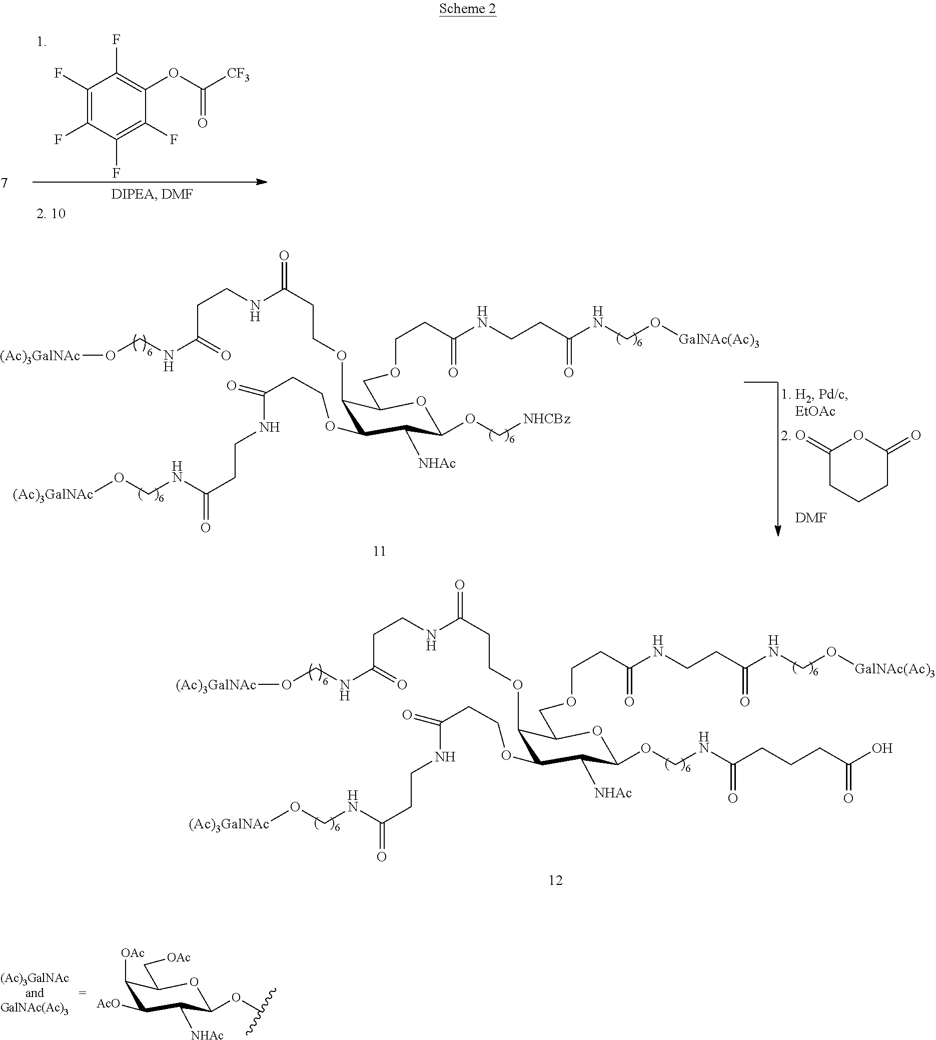

Preparation of compound 11: compound 7 (1.0 equiv.) may be dissolved in CH2012 at 0-10° C. To this solution of compound 7, DIPEA (8 equiv.) and perfluorophenyl trifluoroacetate (4 equiv.) may be added. The resulting mixture may be stirred for 2 hours at 0-10° C. and may be washed with water at 0-10° C., and the separated organic phase may be dried over Na₂SO₄ (200% (w/w)). The organic phase may be cooled to 0-10° C., DIPEA (3 equiv.) may be added, compound 10 (3.4 equiv.) in CH₂Cl₂ may be added dropwise, and the resulting mixture may be stirred for 1 hour at 0-10° C. The reaction mixture may be washed with saturated aqueous NH₄Cl at 0-10° C., phases may be separate, and the organic phase may be washed with water, dried over Na₂SO₄ (200% (w/w)), filtered, and concentrated. To the concentrated filtrate, MTBE may be added to precipitate the solid from the remaining CH₂Cl₂/MTBE.

Removal of the CBz protecting group in 11: this reaction may be performed under the same hydrogenation conditions as those described for the preparation of compound 10, with the exception that the crude product may be dissolved in CH2012. The resulting solution may be added dropwise to MTBE to precipitate solid product, which may be filtered. The filter cake may then be combined with 50% (w/w) Al₂O₃ in CH₂Cl₂ at 20-25° C. for 30 minutes. The resulting mixture may be filtered, and the filtrate may be dried to give the desired product as a solid.

Preparation of compound 12: the product of CBz removal from 11 may be dissolved in DMF and stirred at room temperature for 4 hours with glutaric anhydride. The reaction mixture may be washed with saturated aqueous NaHCO₃, layers may be separated, and the organic phase may be washed with CH₂Cl₂. The resulting solution may be dried in vacuo to give the product.

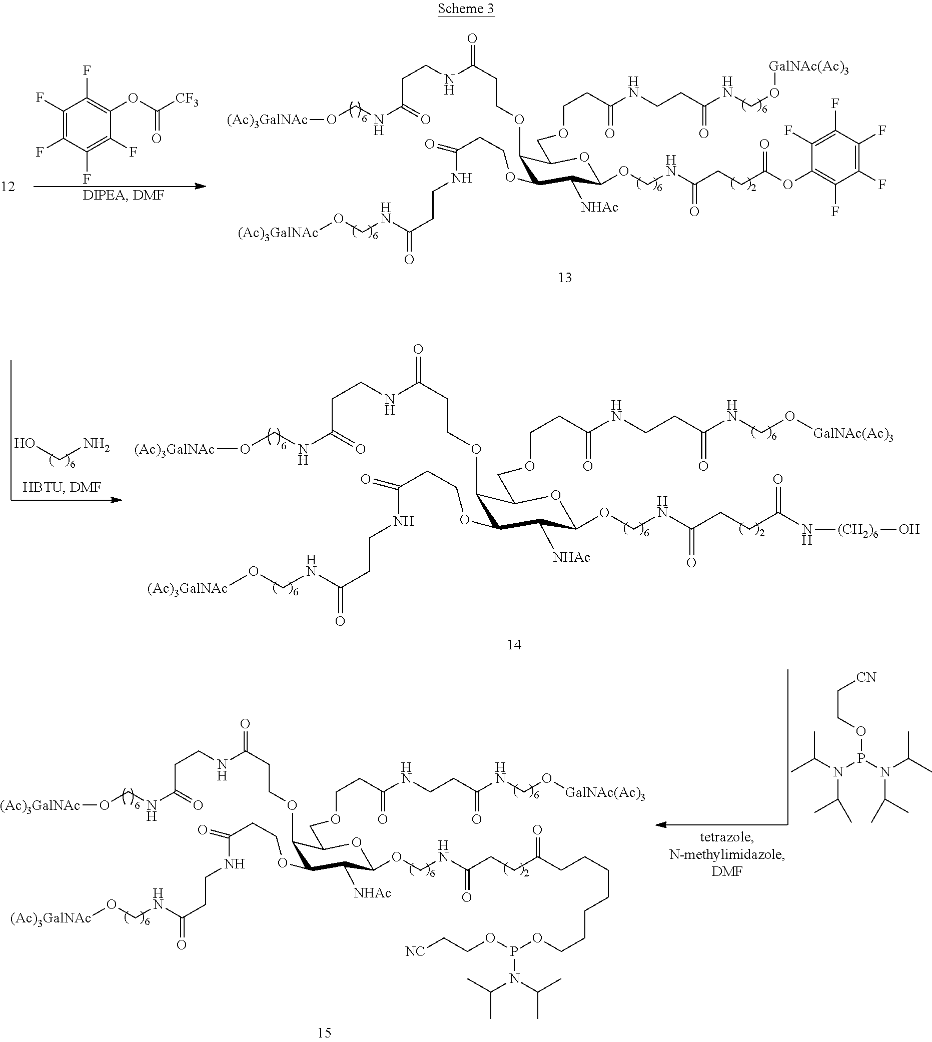

Preparation of compound 13: compound 12 (1 equiv.) may be dissolved in CH₂Cl₂ at 0-10° C. DIPEA (2.0 equiv.) and perfluorophenyl trifluoroacetate (1.5 equiv.) may be added. The reaction mixture may be stirred for 2 hours at 0-10° C. and washed with water at 0-10° C., and the separated organic phase may be dried over Na₂SO₄ (200% (w/w)) and filtered. The filtrate may be concentrated, and the product may be isolated as a solid from CH₂Cl₂/MTB.

Preparation of compound 14: compound 12 (1.0 equiv.) and HBTU (1.1 equiv.) may be dissolved in CH₂Cl₂. The resulting solution may be stirred and cooled to 0-10° C. DIPEA (1.5 equiv.) may be added, and the resulting mixture may be stirred at 0-10° C. for 15 minutes, at which time, 6-amino-1-hexanol (1.05 equiv.) in CH₂Cl₂ may be added dropwise, and the reaction mixture may be stirred for 1 hour at 0-10° C. CH₂Cl₂ may be added to the reaction mixture, followed by the addition of aqueous saturated NH₄C₁ at 0-10° C. Layers may be separated, and the organic phase may be washed with NH₄Cl, dried over Na₂SO₄ (200% (w/w)), filtered, and concentrated. To the concentrated filtrate, MTBE may be added to precipitate the solid from CH₂Cl₂/MTBE. The resulting mixture may be filtered, and the filter cake may be dissolved in CH₂Cl₂. To the resulting solution, Al₂O₃ (100% (w/w)) may be added, and the resulting mixture may be stirred for an hour, at which time, the mixture may be filtered, and the filtrate may be dried in vacuo to give the product as a solid.

Preparation of compound 15: Compound 14 (1.0 equiv.), N-methylimidazole (0.2 equiv.), and tetrazole (0.8 equiv.) may be dissolved in DMF. The resulting solution may be stirred and cooled to 0-10° C. 2-Cyanoethyl-N,N,N′,N′-tetraisopropylphosphordiamidite (3.0 equiv.) may be added dropwise, and the resulting mixture may be stirred for 1 hour at room temperature. The reaction may be quenched by the dropwise addition of water at 0-10° C. Saturated aqueous NaCl and EtOAc may be added at 0-10° C. Layers may be separated, and the aqueous phase may be extracted with EtOAc twice. The organic phase may be dried over Na₂SO₄, filtered, and concentrated. The product may be isolated as a solid by precipitation from CH₂Cl₂/MTBE.

Compound 13 and compound 15 may be used in the preparation of compounds of the invention described herein.

### Example 6. Preparation of an Exemplary Targeting Moiety

Compound 15 from Example 5 may be coupled to an oligonucleotide to produce compound 16.

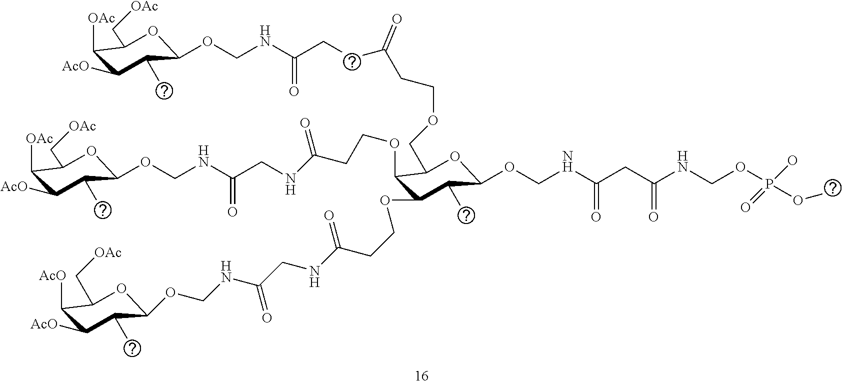

For example, reaction between compound 15 and oligo-O—P(O)(OH)—O—(CH₂)₆—NH₂, or a salt thereof, in buffered medium (e.g., sodium tetraborate buffer at pH 8.5) may produce compound 16.

### Example 7. Preparation of an Exemplary Targeting Moiety

Additionally, a targeting moiety may be prepared as shown in Scheme 4 and described below, e.g., from compound 11 in Example 5.

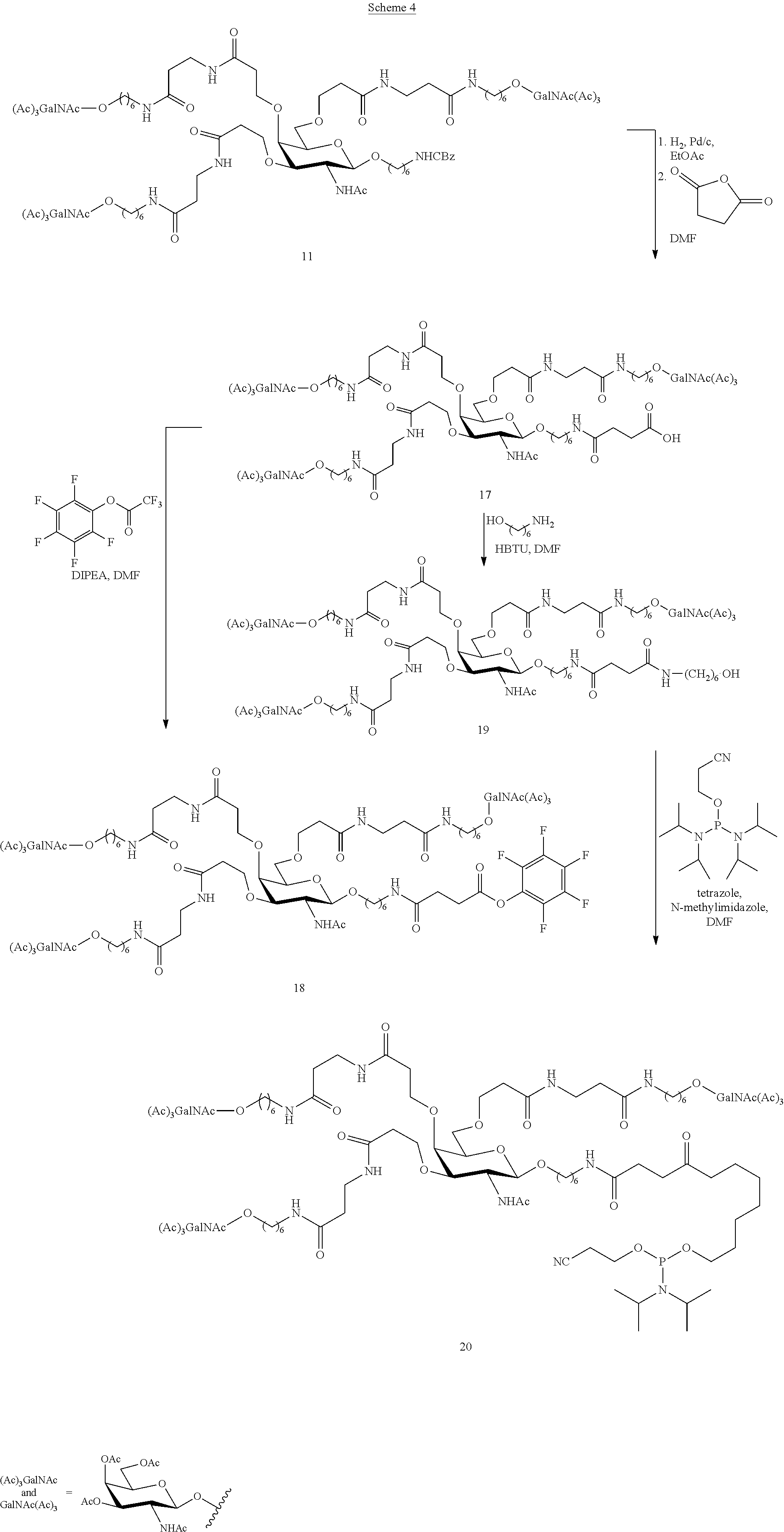

Removal of the CBz protecting group in 11: this reaction may be performed under the same hydrogenation conditions as those described for the preparation of compound 10, with the exception that the crude product may be dissolved in CH2012. The resulting solution may be added dropwise to MTBE to precipitate solid product, which may be filtered. The filter cake may then be combined with 50% (w/w) Al₂O₃ in CH₂Cl₂ at 20-25° C. for 30 minutes. The resulting mixture may be filtered, and the filtrate may be dried to give the desired product as a solid.

Preparation of compound 17: the product of CBz removal from 11 may be dissolved in DMF and stirred at room temperature for 4 hours with succinic anhydride. The reaction mixture may be washed with saturated aqueous NaHCO₃, layers may be separated, and the organic phase may be washed with CH₂Cl₂. The resulting solution may be dried in vacuo to give the product.

Preparation of compound 18: compound 17 (1 equiv.) may be dissolved in CH₂Cl₂ at 0-10° C. DIPEA (2.0 equiv.) and perfluorophenyl trifluoroacetate (1.5 equiv.) may be added. The reaction mixture may be stirred for 2 hours at 0-10° C. and washed with water at 0-10° C., and the separated organic phase may be dried over Na₂SO₄ (200% (w/w)) and filtered. The filtrate may be concentrated, and the product may be isolated as a solid from CH₂Cl₂/MTBE.

Preparation of compound 19: compound 17 (1.0 equiv.) and HBTU (1.1 equiv.) may be dissolved in CH₂Cl₂. The resulting solution may be stirred and cooled to 0-10° C. DIPEA (1.5 equiv.) may be added, and the resulting mixture may be stirred at 0-10° C. for 15 minutes, at which time, 6-amino-1-hexanol (1.05 equiv.) in CH₂Cl₂ may be added dropwise, and the reaction mixture may be stirred for 1 hour at 0-10° C. CH₂Cl₂ may be added to the reaction mixture, followed by the addition of aqueous saturated NH₄Cl at 0-10° C. Layers may be separated, and the organic phase may be washed with NH₄Cl, dried over Na₂SO₄ (200% (w/w)), filtered, and concentrated. To the concentrated filtrate, MTBE may be added to precipitate the solid from CH₂Cl₂/MTBE. The resulting mixture may be filtered, and the filter cake may be dissolved in CH₂Cl₂. To the resulting solution, Al₂O₃ (100% (w/w)) may be added, and the resulting mixture may be stirred for an hour, at which time, the mixture may be filtered, and the filtrate may be dried in vacuo to give the product as a solid.

Preparation of compound 20: Compound 19 (1.0 equiv.), N-methylimidazole (0.2 equiv.), and tetrazole (0.8 equiv.) may be dissolved in DMF. The resulting solution may be stirred and cooled to 0-10° C. 2-Cyanoethyl-N,N,N′,N′-tetraisopropylphosphordiamidite (3.0 equiv.) may be added dropwise, and the resulting mixture may be stirred for 1 hour at room temperature. The reaction may be quenched by the dropwise addition of water at 0-10° C. Saturated aqueous NaCI and EtOAc may be added at 0-10° C. Layers may be separated, and the aqueous phase may be extracted with EtOAc twice. The organic phase may be dried over Na₂SO₄, filtered, and concentrated. The product may be isolated as a solid by precipitation from CH₂Cl₂/MTBE.

Compound 18 and compound 20 may be used in the preparation of compounds of the invention described herein.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

What is claimed is:

1. An antisense oligonucleotide comprising a targeting moiety covalently linked to a nucleobase sequence at least 70% complementary to a LIPA pre-mRNA target sequence in a 5′-flanking intron, a 3′-flanking intron, or a combination of an exon and the 5′-flanking or 3′-flanking intron.

2. The antisense oligonucleotide of claim 1, wherein the antisense oligonucleotide comprises at least 12 nucleosides.

3. The antisense oligonucleotide of claim 2, wherein the antisense oligonucleotide comprises at least 16 nucleosides.

4. The antisense oligonucleotide of any one of claims 1 to 3, wherein the antisense oligonucleotide comprises a total of 50 nucleosides or fewer.

5. The antisense oligonucleotide of any one of claims 1 to 3, wherein the antisense oligonucleotide comprises a total of 30 nucleosides or fewer.

6. The antisense oligonucleotide of any one of claims 1 to 3, wherein the antisense oligonucleotide comprises a total of 20 nucleosides or fewer.

7. The antisense oligonucleotide of any one of claims 1 to 3, wherein the antisense oligonucleotide comprises a total of 16 to 20 nucleosides.

8. An antisense oligonucleotide comprising a total of 20 to 30 nucleosides in a nucleobase sequence at least 70% complementary to a LIPA pre-mRNA target sequence in a 5′-flanking intron, a 3′-flanking intron, or a combination of an exon and the 5′-flanking or 3′-flanking intron.

9. The antisense oligonucleotide of any one of claims 1 to 8, wherein the LIPA target sequence is in a 5′-flanking intron adjacent to exon 8, 3′-flanking intron adjacent to exon 8, or a combination of exon 8 and the adjacent 5′-flanking or 3′-flanking intron.

10. The antisense oligonucleotide of any one of claims 1 to 8, wherein the LIPA target sequence reduces the binding of a splicing factor to an intronic splicing silencer in the 5′-flanking or 3′-flanking intron.

11. The antisense oligonucleotide of any one of claims 1 to 8, wherein the LIPA target sequence comprises at least one nucleotide located among positions 34222-34321 in SEQ ID NO: 1.

12. The antisense oligonucleotide of any one of claims 1 to 8, wherein the LIPA target sequence comprises at least one nucleotide located among positions 34394-34493 in SEQ ID NO: 1.

13. The antisense oligonucleotide of any one of claims 1 to 8, wherein the LIPA target sequence comprises at least one nucleotide located among positions 34398-34480 in SEQ ID NO: 1.

14. The antisense oligonucleotide of any one of claims 1 to 8, wherein the LIPA target sequence comprises at least one nucleotide located among positions 34401-34422 in SEQ ID NO: 1.

15. The antisense oligonucleotide of any one of claims 1 to 8, wherein the LIPA target sequence comprises at least one nucleotide located among positions 34456-34473 in SEQ ID NO: 1.

16. The antisense oligonucleotide of any one of claims 1 to 8, wherein the nucleobase sequence is complementary to a sequence within the 5′-flanking intron of the pre-mRNA.

17. The antisense oligonucleotide of any one of claims 1 to 8, wherein the LIPA target sequence is located within the 5′-flanking intron among positions up to 34321 in SEQ ID NO: 1.

18. The antisense oligonucleotide of any one of claims 1 to 7, wherein the nucleobase sequence has at least 70% sequence identity to SEQ ID NO: 68, 81, or 98.

19. The antisense oligonucleotide of any one of claims 1 to 8, wherein the LIPA target sequence is located within the 3′-flanking intron of the pre-mRNA.

20. The antisense oligonucleotide of any one of claims 1 to 8, wherein the LIPA target sequence is located within the 3′-flanking intron among positions up to 34500 in SEQ ID NO: 1.

21. The antisense oligonucleotide of any one of claims 1 to 7, wherein the nucleobase sequence has at least 70% sequence identity to any one of SEQ ID NOs: 7, 9-15, 22-26, 29, 32, 34-41, 45-49, 51, 54, 56-60, 62-64, 67, 70-72, 74-80, 83-86, 88 and 89.

22. The antisense oligonucleotide of any one of claims 1 to 8, wherein the LIPA target sequence is located among positions 34394 to 34498 in SEQ ID NO: 1.

23. The antisense oligonucleotide of any one of claims 1 to 7, wherein the nucleobase sequence has at least 70% sequence identity to SEQ ID NO: 7, 9, 12, 13, 15, 22, 23, 24, 25, 26, 32, 34, 35, 36, 38, 39, 40, 41, 45, 47, 48, 49, 51, 54, 56, 57, 58, 59, 62, 63, 64, 70, 71, 74, 75, 76, 77, 78, 79, 80, 83, 84, 85, 86, 88, or 89.

24. The antisense oligonucleotide of any one of claims 1 to 7, wherein the nucleobase sequence has at least 70% sequence identity to SEQ ID NO: 84.

25. The antisense oligonucleotide of any one of claims 1 to 7, wherein the nucleobase sequence has at least 70% sequence identity to SEQ ID NO: 26.

26. The antisense oligonucleotide of any one of claims 1 to 7, wherein the nucleobase sequence has at least 70% sequence identity to SEQ ID NO: 22.

27. The antisense oligonucleotide of any one of claims 1 to 7, wherein the nucleobase sequence has at least 70% sequence identity to SEQ ID NO: 85.

28. The antisense oligonucleotide of any one of claims 1 to 7, wherein the nucleobase sequence has at least 70% sequence identity to SEQ ID NO: 76.

29. The antisense oligonucleotide of any one of claims 1 to 7, wherein the nucleobase sequence has at least 70% sequence identity to SEQ ID NO: 41.

30. The antisense oligonucleotide of any one of claims 1 to 7, wherein the nucleobase sequence has at least 70% sequence identity to SEQ ID NO: 56.

31. The antisense oligonucleotide of any one of claims 1 to 7, wherein the nucleobase sequence has at least 70% sequence identity to SEQ ID NO: 23.

32. The antisense oligonucleotide of any one of claims 1 to 7, wherein the nucleobase sequence has at least 70% sequence identity to SEQ ID NO: 79.

33. The antisense oligonucleotide of any one of claims 1 to 7, wherein the nucleobase sequence has at least 70% sequence identity to SEQ ID NO: 59.

34. The antisense oligonucleotide of any one of claims 1 to 7, wherein the nucleobase sequence has at least 70% sequence identity to SEQ ID NO: 58.

35. The antisense oligonucleotide of any one of claims 1 to 7, wherein the nucleobase sequence has at least 70% sequence identity to SEQ ID NO: 34.

36. The antisense oligonucleotide of any one of claims 1 to 7, wherein the nucleobase sequence has at least 70% sequence identity to SEQ ID NO: 54.

37. The antisense oligonucleotide of claim 12, 19, 20, 21, or 22, wherein the 5′-terminal nucleotide of the oligonucleotide is complementary to the LIPA pre-mRNA at any one position selected from the group consisting of 34394-34398 in SEQ ID NO: 1.

38. The antisense oligonucleotide of any one of claims 1 to 7, wherein the nucleobase sequence has at least 70% sequence identity to any one of SEQ ID NOs: 7, 22, 23, 24, 26, 32, 34, 38, 41, 49, 56, 58, 59, 63, 70, 71, 75, 76, 79, 80, 84, 85, 86, and 88.

39. The antisense oligonucleotide of any one of claims 1 to 38, wherein the sequence identity is at least 90%.

40. The antisense oligonucleotide of claim 39, wherein the sequence identity is 100%.

41. The antisense oligonucleotide of any one of claims 1 to 40, wherein the antisense oligonucleotide comprises at least one modified nucleobase.

42. The antisense oligonucleotide of any one of claims 1 to 41, wherein the antisense oligonucleotide comprises at least one modified internucleoside linkage.

43. The antisense oligonucleotide of claim 42, wherein the modified internucleoside linkage is a phosphorothioate linkage.

44. The antisense oligonucleotide of claim 42, wherein the phosphorothioate linkage is a stereochemically enriched phosphorothioate linkage.

45. The antisense oligonucleotide of any one of claims 42 to 44, wherein at least 50% of internucleoside linkages in the antisense oligonucleotide are independently the modified internucleoside linkage.

46. The antisense oligonucleotide of claim 45, wherein at least 70% of internucleoside linkages in the antisense oligonucleotide are independently the modified internucleoside linkage.

47. The antisense oligonucleotide of claim 46, wherein all internucleoside linkages in the antisense oligonucleotide are independently the modified internucleoside linkage.

48. The antisense oligonucleotide of any one of claims 1 to 47, wherein the antisense oligonucleotide comprises at least one modified sugar nucleoside.

49. The antisense oligonucleotide of claim 48, wherein at least one modified sugar nucleoside is a 2′-modified sugar nucleoside.

50. The antisense oligonucleotide of claim 49, wherein at least one 2′-modified sugar nucleoside comprises a 2′-modification selected from the group consisting of 2′-fluoro, 2′-methoxy, and 2′-methoxyethoxy.

51. The antisense oligonucleotide of claim 50, wherein the 2′-modified sugar nucleoside comprises the 2′-methoxyethoxy modification.

52. The antisense oligonucleotide of any one of claims 48 to 51, wherein at least one modified sugar nucleoside is a bridged nucleic acid.

53. The antisense oligonucleotide of claim 52, wherein the bridged nucleic acid is a locked nucleic acid (LNA), ethylene-bridged nucleic acid (ENA), or cEt nucleic acid.

54. The antisense oligonucleotide of any one of claims 48 to 53, wherein all nucleosides in the antisense oligonucleotide are independently the modified sugar nucleosides.

55. The antisense oligonucleotide of any one of claims 1 to 41, wherein the antisense oligonucleotide is a morpholino oligomer.

56. The antisense oligonucleotide of any one of claims 1 to 55, wherein the targeting moiety is covalently conjugated at the 5′-terminus of the antisense oligonucleotide.

57. The antisense oligonucleotide of any one of claims 1 to 55, wherein the targeting moiety is covalently conjugated at the 3′-terminus of the antisense oligonucleotide.

58. The antisense oligonucleotide of any one of claims 1 to 55, wherein the targeting moiety is covalently conjugated at an internucleoside linkage of the antisense oligonucleotide.

59. The antisense oligonucleotide of any one of claims 1 to 58, wherein the targeting moiety is covalently conjugated through a linker.

60. The antisense oligonucleotide of claim 59, wherein the linker is a cleavable linker.

61. The antisense oligonucleotide of any one of claims 1 to 60, wherein the targeting moiety comprises N-acetylgalactosamine.

62. The antisense oligonucleotide of claim 61, wherein the targeting moiety is an N-acetylgalactosamine cluster.

63. The antisense oligonucleotide of claim 62, wherein the N-acetylgalactosamine cluster is of the following structure: 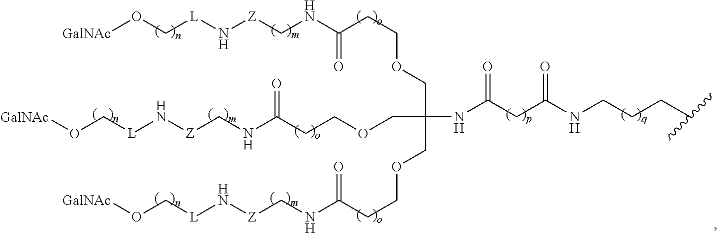
wherein
each L is independently CO or CH₂,
each Z is independently CO or CH₂,
each n is independently 1 to 9,
each m is independently 1 to 5,
each o is independently 0 to 1,
each p is independently 1 to 10, and
each q is independently 1 to 10.

64. The antisense oligonucleotide of claim 63, wherein each L is CH₂.

65. The antisense oligonucleotide of claim 63 or 64, wherein each Z is CO.

66. The antisense oligonucleotide of any one of claims 63 to 65, wherein each n is 5.

67. The antisense oligonucleotide of any one of claims 63 to 66, wherein each m is 2.

68. The antisense oligonucleotide of any one of claims 63 to 67, wherein each o is 1.

69. The antisense oligonucleotide of any one of claims 63 to 68, wherein each p is 2.

70. The antisense oligonucleotide of any one of claims 63 to 68, wherein each p is 3.

71. The antisense oligonucleotide of any one of claims 63 to 65, wherein each q is 4.

72. The antisense oligonucleotide of claim 63, wherein the N-acetylgalactosamine cluster is of the following structure:

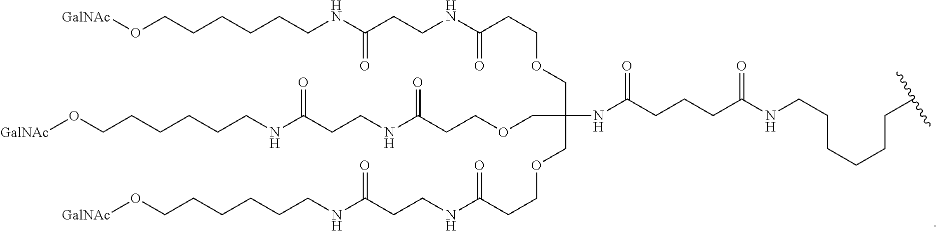

73. A pharmaceutical composition comprising the antisense oligonucleotide of any one of claims 1 to 72 and a pharmaceutically acceptable excipient.

74. A method of increasing the level of exon-containing LIPA mRNA molecules in a cell expressing an aberrant LIPA gene, the method comprising contacting the cell with the antisense oligonucleotide of any one of claims 1 to 72.

75. The method of claim 74, wherein the cell is in a subject.

76. The method of claim 75, wherein the cell is a hepatocyte.

77. The method of claim 75, wherein the cell is a Kupffer cell.

78. A method of treating Wolman Disease or Cholesteryl Ester Storage Disease in a subject having an aberrant LIPA gene, the method comprising administering a therapeutically effective amount of the antisense oligonucleotide of any one of claims 1 to 72 or the pharmaceutical composition of claim 73 to the subject in need thereof.

79. The method of claim 78, wherein the administering step is performed parenterally.

80. The method of claim 78 or 79, further comprising administering to the subject a therapeutically effective amount of a second therapy for Wolman Disease or Cholesteryl Ester Storage Disease.

81. The method of claim 80, wherein the second therapy is a recombinant lysosomal acid lipase or a statin or a salt thereof.

82. The method of claim 80, wherein the second therapy is a hematopoietic stem cell transplantation.

83. The method of any one of claims 76 to 82, wherein the therapeutically effective amount is 1 mg/kg to 10 mg/kg.

84. The method of any one of claims 76 to 83, wherein the antisense oligonucleotide or the pharmaceutical composition is administered from once monthly to once weekly.

85. The method of any one of claims 76 to 83, wherein the antisense oligonucleotide or the pharmaceutical composition is administered once weekly, biweekly, or monthly.

86. The method of any one of claims 74 to 85, wherein the aberrant LIPA gene is LIPA having a g.34393G>A mutation in SEQ ID NO: 1.
